(19) United States
(12) Patent Application Publication
(10) Pub. No.: US 2021/0364495 A1
(43) Pub. Date: Nov. 25, 2021

(54) CRISPR ASSOCIATED PROTEIN REACTIVE T CELL IMMUNITY

(71) Applicant: CHARITÉ-UNIVERSITÄTSMEDIZIN BERLIN, Berlin (DE)

(72) Inventors: Leila AMINI, Berlin (DE); Petra REINKE, Berlin (DE); Michael SCHMÜCK-HENNERESSE, Berlin (DE); Hans-Dieter VOLK, Berlin (DE); Dimitrios WAGNER, Berlin (DE); Desiree WENDERING, Berlin (DE)

(73) Assignee: CHARITÉ-UNIVERSITÄTSMEDIZIN BERLIN, Berlin (DE)

(21) Appl. No.: 17/040,058

(22) PCT Filed: Mar. 22, 2020
(86) PCT No.: PCT/EP2019/057317

§ 371 Date: Sep. 22, 2020

(30) Foreign Application Priority Data
Mar. 22, 2018 (EP) 18163491.6
Mar. 23, 2018 (EP) 18163801.6
Sep. 18, 2018 (EP) 18195296.1

(51) Int. Cl. G01N 33/50; C12N 5/0783

(52) U.S. Cl. CPC G01N 33/505; C12N 5/0637

(57) ABSTRACT

The invention relates to a method for determining T cell mediated immunity towards a CRISPR associated protein by contacting said cell preparation obtained from a patient with a CRISPR associated protein or a peptide mix that represents its amino acid sequence, or a cell manipulated to contain a CRISPR protein polypeptide to provide activated T cells. Subsequently, one or more subpopulations of said activated T cells are marked by specific ligand and counted, and a ratio (T_{REG}/T_{EFF}) of activated regulatory T cells to activated effector T cells is used to assess the immune status of the patient with respect to the CRISPR associated protein. The invention further relates to a method for generating a CRISPR associated protein specific Treg population and its use in therapy.

The present invention relates to methods of diagnosing T cell mediated immunity to CRISPR associated proteins in a patient, and to regulatory T cell preparations for use in CRISPR therapy.

### BACKGROUND

SpCas9 (Cas 9 enzyme from Streptococcus (S.) pyogenes) was the first Clustered Regularly Interspaced Short Palindromic Repeats (CRISPR) associated nuclease hijacked to introduce DNA double-strand breaks at specific DNA sequences. Through the ease of target adaption and the remarkable efficacy, it advanced to the most popular tool for re-writing genes in research and potential clinical applications. The major concern for clinical translation of CRISPR/Cas9 technology is the risk for off-target activity causing potentially harmful mutations or chromosomal aberrations. High-fidelity Cas9 enzymes were developed to reduce the probability of these events. Furthermore, novel Cas9-based fusion proteins allow base editing or specific epigenetic reprogramming without inducing breaks in the DNA.

Most approaches are based on the original SpCas9 enzyme that originates in the facultatively pathogenic bacterium S. pyogenes.

Approximately 12% of children under 18 have an asymptomatic colonization of the faucial mucosa with S. pyogenes 12. S. pyogenes-associated pharyngitis and pyoderma are among the most common diseases related to S. pyogenes infection worldwide. Considering the high prevalence of S. pyogenes infection, the inventors hypothesized that SpCas9 could elicit an adaptive memory immune response in humans. Most therapeutic applications aim to temporarily express Cas9 nuclease or deliver the protein directly into the target cells. Thus, SpCas9-specific antibodies may be negligible. However, intracellular protein degradation processes lead to peptide presentation of Cas9 fragments on the cellular surface of gene-edited cells that may be recognized by SpCas9-reactive T cells.

A pre-existing T cell immunity, particularly if tissue-migrating effector T (T_{EFF}) cells are present, would result in a fast inflammatory and cytotoxic response to cells presenting Cas9 peptides on their major histocompatibility complexes (MHC)-molecules during or after intra-tissue gene editing.

Immunocompetent mice treated with CRISPR/Cas9-encoding vectors exhibit humoral and cellular immune responses against the Cas9 protein, that impact the efficacy of treatment and can cause tissue damage. Most applications aim to express the Cas9 nuclease in or deliver it directly to the target cell. Intracellular protein degradation processes lead to peptide presentation of Cas9 fragments on the cellular surface of gene-edited cells that may be recognized by T cells. Even if this might be less relevant for a primary T-cell response which can easily be prevented or delayed and temporary Cas9-expression is sufficient in many approaches, a pre-existing memory would have major impact.

The present invention provides a solution to overcome the problem of this pre-existing immunity by adoptive CRISPR-associated-protein-specific regulatory T cell (T_{REG}) therapy comprising methods of determining and assessing T_{REG} and T_{EFF} cells obtained from a patient, providing a preparation of T_{REG} cells specifically reactive to CRISPR associated protein polypeptides and a method of producing such preparation. Furthermore, the invention provides a method for assessing a patient's immune reactivity to ex-vivo CRISPR/Cas-edited cells prior to their administration.

### DESCRIPTION

Based on the above-mentioned state of the art, the objective of the present invention is to provide means and methods to overcome and possibly counteract a pre-existing CRISPR-associated-protein-specific immunity in a patient prior, or subsequent, to in vivo CRISPR/Cas-based gene therapy, and to assess the immunogenicity of ex-vivo CRISPR/Cas-edited cells prior to administration to a patient. This objective is attained by the claims of the present specification.

### Terms and Definitions

The term polypeptide in the context of the present specification relates to a molecule consisting of 50 or more amino acids that form a linear chain wherein the amino acids are connected by peptide bonds. The amino acid sequence of a polypeptide may represent the amino acid sequence of a whole (as found physiologically) protein or fragments thereof.

The term peptide in the context of the present specification relates to a molecule consisting of up to 50 amino acids, in particular 8 to 30 amino acids, more particularly 8 to 15 amino acids, that form a linear chain wherein the amino acids are connected by peptide bonds.

In the context of the present specifications the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1-2; Gap costs: Linear. Unless otherwise stated, sequence identity values provided herein refer to the value obtained using the BLAST suite of programs (Altschul et al., J. Mol. Biol. 215:403-410 (1990)) using the above identified default parameters for protein and nucleic acid comparison, respectively.

The term CRISPR associated protein in the context of the present specification relates to a CRISPR associated protein originating from bacteria as specified in Shmakov et al., Nature Reviews Microbiology (2017) 15, 169-182), particularly to a CRISPR associated protein from S. pyogenes, S. aureus, C. jejuni, N. meningitides, Acidaminococcus or Lachnospiracea.

The term CRISPR associated protein polypeptide in the context of the present specification relates to a polypeptide, the amino acid sequence of which is at least 85% [particularly ≥90%, ≥92%, ≥94, ≥96%, ≥98%] identical to the amino acid sequence of a functional CRISPR associated protein and functions in the CRISPR gene editing system. The CRISPR associated protein polypeptide may be a native polypeptide or a recombinant polypeptide. The term CRISPR associated protein polypeptide encompasses fully the definition of CRISPR associated protein given above. Furthermore, fusion proteins comprising a functional CRISPR associated protein as per the above definition associated to another enzymatic function, for example an enzymatic function useful in modifying genetic information inside a cell, are encompassed. One example of such fusion protein is given in Komor et al., Nature. 2016 May 19; 533(7603):420-4. doi: 10.1038/nature17946. For determining sequence identity in case of a fusion protein comprising a functional CRISPR associated protein, only the protein sequence of the functional CRISPR associated protein domain is considered.

The term Cas9 polypeptide in the context of the present specification relates to a polypeptide the amino acid sequence of which is at least 85% [particularly ≥90%, ≥92%, ≥94, ≥96%, ≥98%] identical to the amino acid sequence of a functional Cas9 protein, particularly the Cas9 protein from Streptococcus pyogenes serotype M1 (SpCas9; NBI Gene ID: 901176; Uniprot Entry ID: Q99ZW2; Uniprot Entry name: CAS9_STRP1), the Cas protein from Streptococcus thermophilus (NCBI Gene ID: 31939158; Uniprot Entry: G3ECR1; Uniprot Entry name: CAS9_STRTR), the Cas9 protein from Staphylococcus aureus (Uniprot Entry: J7RUA5; Uniprot Entry name: CAS9_STAAU), the Cas9 protein from Campylobacter jejuni (NCBI Gene ID: 905809; Uniprot Entry: Q0P897; Uniprot Entry Name: CAS9_CAMJE) or the Cas 9 protein from Neisseria meningitidis (Uniprot Entry: A1IQ68; Uniprot Entry Name: CAS9_NEIMA). The Cas9 polypeptide may be a Cas9 polypeptide substantially identical to the protein found in nature, or a Cas9 polypeptide having ≥85% sequence identity to the Cas9 protein found in nature and having substantially the same biological activity.

The term Cas12 polypeptide in the context of the present specification relates to a polypeptide the amino acid sequence of which is at least 85% [particularly ≥90%, 92%, ≥94, ≥96%, ≥98%] identical the amino acid sequence of a functional Cas12 protein, particularly the Cas12a/Cpf1 protein from Acidaminococcus sp. strain BV3L6 (Uniprot Entry: U2UMQ6; Uniprot Entry Name: CS12A_ACISB) or the Cas12a/Cpf1 protein from Francisella tularensis (Uniprot Entry: A0Q7Q2; Uniprot Entry Name: CS12A_FRATN). The Cas12 polypeptide may be a Cas12 polypeptide substantially identical to the protein found in nature, or a Cas12 polypeptide having ≥85% sequence identity to the Cas12 protein found in nature and having substantially the same biological activity.

The term having substantially the same biological activity in the context of the present invention relates to either one or both main functions of a CRISPR associated protein, i.e. endonuclease activity and CRISPR-RNA (crRNA) mediated DNA binding. Particularly in the case of a fusion protein, the Cas protein domain may perform crRNA mediated DNA binding but is catalytically inactive with respect to the endonuclease activity.

The term homologue in the context of the present specification relates to a functional polypeptide having a sequence identity of 85% or more with a CRISPR associated protein, in particular a protein with an amino acid sequence referred to as Q99ZW2, G3ECR1, J7RUA5, Q0P897, A1IQ68 U2UMQ6 or A0Q7Q2 (Uniprot Entry IDs).

The term plurality of peptides in the context of the present specification relates to a peptide mix of overlapping peptide sequences covering entire immunogenic antigens from CRISPR associated proteins from bacteria. A peptide of the peptide mix consists of up to 50 amino acids, in particular 8 to 30 amino acids, more particularly 8 to 15 amino acids. For example, one peptide is characterized by an amino acid sequence length n. The amino acid sequence of this peptide overlaps with the amino acid sequence of another peptide of the peptide mix by n-k amino acids, wherein k is an integer between 1 and 4. The sequence fragment of non-overlapping amino acids (k) may overlap with yet another amino acid sequence of the peptide mix. The plurality of peptides may be obtained applying methods such as recombinant expression or synthetic peptide synthesis or by endogenously antigen processing within antigen presenting cells. Methods of making and using such plurality of peptides in stimulation of T cells are described in U.S. Pat. No. 8,932,806 (B1) and US2004106159 (A1) which are incorporated herein by reference.

The term molecular probe in the context of the present specification relates to a specific ligand, particularly an antibody, antibody fragment, an antibody-like molecule or aptamer, more particularly an antibody or antibody fragment, that can bind to a target molecule, such as a specific surface protein or a specific transcription factor of a T cell with a dissociation constant of ≤10⁻⁷ mol/l, particularly ≤10⁻⁸ mol/l. The molecular probe comprises a detectable marker such as a particle, bead, dye or enzyme.

The term set of molecular probes relates to a panel of molecular probes for positive and/or negative selection of marker expression.

The term antibody-like molecule in the context of the present specification refers to a molecule capable of specific binding to another molecule or target with high affinity/a Kd≤10E-8 mol/l. An antibody-like molecule binds to its target similarly to the specific binding of an antibody. The term antibody-like molecule encompasses a repeat protein, such as a designed ankyrin repeat protein (Molecular Partners, Zurich), an engineered antibody mimetic proteins exhibiting highly specific and high-affinity target protein binding (see US2012142611, US2016250341, US2016075767 and US2015368302, all of which are incorporated herein by reference). The term antibody-like molecule further encompasses, but is not limited to, a polypeptide derived from armadillo repeat proteins, a polypeptide derived from leucine-rich repeat proteins and a polypeptide derived from tetratricopeptide repeat proteins.

The term antibody-like molecule further encompasses a polypeptide derived from protein A domains, a polypeptide derived from fibronectin domain FN3, a polypeptide derived from consensus fibronectin domains, a polypeptide derived from lipocalins, a polypeptide derived from Zinc fingers, a polypeptide derived from Src homology domain 2 (SH2), a polypeptide derived from Src homology domain 3 (SH3), a polypeptide derived from PDZ domains, a polypeptide derived from gamma-crystallin, a polypeptide derived from ubiquitin, a polypeptide derived from a cysteine knot polypeptide, a polypeptide derived from a knottin, a polypeptide derived from a cystatin, a polypeptide derived from Sac7d, a triple helix coiled coil (also known as alphabodies), a polypeptide derived from a Kunitz domain of a Kunitz-type protease inhibitor and a polypeptide derived from a carbohydrate binding module 32-2.

The term protein A domains derived polypeptide refers to a molecule that is a derivative of protein A and is capable of specifically binding the Fc region and the Fab region of immunoglobulins.

The term armadillo repeat protein refers to a polypeptide comprising at least one armadillo repeat, wherein an armadillo repeat is characterized by a pair of alpha helices that form a hairpin structure.

Subpopulations of T cells relevant for the invention disclosed herein are defined by the expression profile of specific marker molecules. The expression of specific marker molecules may be determined by flow cytometry using appropriate ligands. Marker molecules that are expressed on the surface of T cells may be detected on living T cells as well as on fixated T cells. Marker molecules that are expressed intracellularly, e.g. a transcription factor, may be detected in fixated T cells. Thus, detection of an expression profile described below that comprises the transcription factor FoxP3 may be performed only on T cells that are fixated and thus no longer viable. An alternative transcription factor suitable for detecting non-activated or activated regulatory T cells is the transcription factor helios. Thus, FoxP3 may be replaced by helios in the profiles described below.

The term non-activated regulatory T cell or T_{REG} cell in the context of the present specification relates to a T cell characterized by the following expression profile:

CD3⁺ CD4⁺ CD137⁻ CD25^{high} [CD127⁻ and/or FoxP3⁺].

This means, non-activated regulatory T cells may be detected as follows:
CD3⁺ CD4⁺ CD137⁻ CD25^{high} CD127⁻ FoxP3⁺
CD3⁺ CD4⁺ CD137⁻ CD25^{high} FoxP3⁺
CD3⁺ CD4⁺ CD137⁻ CD25^{high} CD127⁻.

The term activated regulatory T cell or activated T_{REG} cell in the context of the present specification relates to a T cell characterized by the following expression profile:

CD3⁺ CD4⁺ CD137⁺ CD154⁻ CD25^{high} [CD127⁻ and/or FoxP3⁺].

This means, activated regulatory T cells may be detected as follows:
CD3⁺ CD4⁺ CD137⁺ CD154⁻ CD25^{high} CD127⁻ FoxP3⁺
CD3⁺CD4⁺CD137⁺CD154⁻CD25^{high} FoxP3⁺.
CD3⁺ CD4⁺ CD137⁺ CD154⁻ CD25^{high} CD127⁻.

The term non-activated effector T cell or T_{EFF} cell in the context of the present specification relates to a T cell characterized by either one of the following expression profiles (a) or (b):

Profile (a): CD3⁺ CD4⁺ CD25^{low} or CD3⁺ CD4⁺ FoxP3⁻

Profile (b): CD3⁺ CD8⁺ CD25^{low} or CD3⁺ CD8⁺ FoxP3⁻.

For the determination of non-activated effector T cells, T cells can be divided into two subpopulations (CD3⁺ CD4⁺ and CD3⁺ CD8⁺). From these subpopulations, regulatory T cells are depleted using ligands specific to either CD25 (living or fixated cells) or FoxP3 (fixated cells). The term activated effector T cell or activated T_{EFF} cell in the context of the present specification relates to a T cell characterized by either one of the following expression profiles (c), (d) or (e):

Profile (c): CD3⁺ CD4⁺ CD137⁺ CD154⁺

Profile (d): CD3⁺ CD8⁺ CD137⁺

Profile (e): CD3⁺ CD4⁺ CD137⁺ CD25^{low} or CD3⁺ CD4⁺ CD137⁺ FoxP3⁻.

Activated effector T cells can be divided into three subpopulations. The subpopulation of profile (e) may be determined by first detecting CD3⁺ CD4⁺ CD137⁺ cells followed by depletion of regulatory T cells using ligands specific to CD25 (living or fixated cells) and/or FoxP3 (fixated cells).

The expression profiles described above comprise a minimal set of marker molecules. Each expression profile may be expanded by detecting the absence (non-activated T_{REG} or T_{EFF}) or presence (activated T_{REG} or T_{EFF}) of activation-specific marker molecules.

The term activation-specific marker molecule in the context of the present specification relates to molecules produced or expressed by T cells following CRISPR associated protein induced antigenic stimulation. Non-limiting examples may be CD137, CD154, CD69, CD107a, Granzyme B, Perforin, CD25, KLRG1, CD71, CD80, CD86, CD134, HLA-DR, IFNγ, TNFα and IL-2. Depending on the marker molecule, intra- and/or extracellular detections methods may be applied.

In the present specification, the term positive, when used in the context of expression of a marker, refers to an expression assayed by a fluorescent labelled antibody, wherein the fluorescence is at least 30% higher (≥30%), particularly ≥50% or ≥80%, in median fluorescence intensity in comparison to staining with an isotype-matched antibody which does not specifically bind the same target. Such expression of a marker is indicated by a superscript “plus” (⁺), following the name of the marker, e.g. CD4⁺.

In the present specification, the term negative, when used in the context of expression of a marker, refers to an expression assayed by a fluorescent labelled antibody, wherein the median fluorescence intensity is less than 30% higher, particularly less than 15% higher, than the median fluorescence intensity of an isotype-matched antibody which does not specifically bind the same target. Such expression of a marker is indicated by a superscript minus (⁻), following the name of the marker, e.g. CD127⁻.

T cell populations may be distinguished by fluorescence activated cell sorting (FACS) using an antibody such as anti-CD25 antibody. T cells obtained from a human blood sample usually divide into three populations with regard to the expression level of CD25 (high, medium and low expression).

High expression of a marker, for example high expression of CD25, refers to the expression level of such marker in a clearly distinguishable cell population that is detected by FACS showing the highest fluorescence intensity per cell compared to the other populations characterized by a lower fluorescence intensity per cell. A high expression is indicated by superscript “high” or “hi” following the name of the marker, e.g. CD25^{high}. The term “is expressed highly” refers to the same feature.

Low expression of a marker, for example low expression of CD25, refers to the expression level of such marker in a clearly distinguishable cell population that is detected by FACS showing the lowest fluorescence intensity per cell compared to the other populations characterized by higher fluorescence intensity per cell. A low expression is indicated by superscript “low” or “lo” following the name of the marker, e.g. CD25^{low}. The term “is expressed lowly” refers to the same feature.

The expression of a marker may be assayed via techniques such as fluorescence microscopy, flow cytometry, ELISPOT, ELISA or multiplex analyses.

Surface molecule expression may also be assessed by adding detection antibodies to stimulation cultures e.g.: CD154 and CD137. In case CD154 is used, CD154 detection antibody may be added to culture at stimulation initiation or after stimulation. In the latter case, an antibody against CD40 may be added to facilitate CD154 detection.

IL-2 is interleukin 2, such as human IL-2 (Gene ID 3558) or a homologue thereof.

The term mTOR inhibitor in the context of the present specification relates to compounds that selectively bind to the protein referred to as “mammalian target of rapamycin” (mTOR), or to molecular interaction partners of the mTOR complex, thereby decreasing or abolishing its molecular function. The term is not meant to encompass vitamin D or any of its metabolites.

A selective inhibitor of mTOR specifically binds to mTOR Complex 1 (mTORC1). mTORc1 is composed of mTOR (UniProt No. P42345) itself, regulatory-associated protein of mTOR (Raptor; Uniprot No. Q8N122), mammalian lethal with SEC13 protein 8 (MLST8, Uniprot No. Q9BVC4) and the recently identified PRAS40 and DEPTOR (Uniprot No. Q8TB45). mTORc1 (also referred to in the literature as TORC1) is a major component of the PI3K/AKT pathway. The inhibitor of mTOR may be selected from rapamycin (sirolimus, CAS No. 53123-88-9), and the group of rapamycin analogues (rapalogues) characterized by a modification of the oxygen in position 40 of the rapamycin scaffold; particularly from everolimus (RAD001, CAS No. 159351-69-6), temsirolimus (CCI-779, NSC 683864, CAS No. 162635-04-3), 32 deoxy-rapamycin (SAR943, CAS No. 186752-78-3), ridaforolimus (Deforolimus, MK-8669, CAS No. 572924-54-0), Zotarolimus (ABT-578, CAS No. 221877-54-9), and NAB Rapamycin (nanoparticle albumin-bound rapamycin).

Rapamycin and rapalogue mTOR inhibitors act by binding to FKBP12, which in turn forms a ternary complex with mTOR in presence of rapamycin or rapalogues, which has a sub-nanomolar dissociation constant.

The inhibitor of mTOR may be a heteroaromatic kinase inhibitor mTOR inhibitor that shares specificity for mTOR and other PI3K molecules, which shall be referred to as PI3 kinase inhibitors herein. A PI3 kinase inhibitor is selected from the group consisting of
PI-103 (3-(4-morpholinopyrido[3′,2′:4,5]furo[3,2-d]pyrimidin-2-yl)phenol; CAS No. 71935-74-9),
apitolisib ((2S)-1-[4-[[2-(2-aminopyrimidin-5-yl)-7-methyl-4-morpholin-4-ylthieno[3,2-d]pyrimidin-6-yl]methyl]piperazin-1-yl]-2-hydroxypropan-1-one;
GDC-0980, RG7422, CAS No. 1032754-93-0),
Dactolisib (2-methyl-2-(4-(3-methyl-2-oxo-8-(quinolin-3-yl)-2,3-dihydro-1H-imidazo[4,5-c]quinolin-1-yl)phenyl)propanenitrile;
NVP-BEZ235 (2-methyl-2-(4-(3-methyl-2-oxo-8-(quinolin-3-yl)-2,3-dihydro-1H-imidazo[4,5-c]quinolin-1-yl)phenyl)propanenitrile; BEZ235, CAS No. 915019-65-7),
BGT226 ((Z)-but-2-enedioic acid; 8-(6-methoxypyridin-3-yl)-3-methyl-1-[4-piperazin-1-yl-3-(trifluoromethyl)phenyl]imidazo[4,5-c]quinolin-2-one; NVP-BGT226, CAS No. 1245537-68-1),
Omipalisib (2,4-difluoro-N-[2-methoxy-5-(4-pyridazin-4-ylquinolin-6-yl)pyridin-3-yl]benzenesulfonamide; GSK2126458, GSK458, CAS No. 1086062-66-9),
voxtasilib (XL765, 2-amino-8-ethyl-4-methyl-6-(1H-pyrazol-5-yl)pyrido[2,3-d]pyrimidin-7(8H)-one; SAR245409; CAS No. 934493-76-2) and
“Voxtasilib analogue” (N-[4-[[3-(3,5-dimethoxyanilino)quinoxalin-2-yl]sulfamoyl]phenyl]-3-methoxy-4-methylbenzamide, CAS No. 1349796-36-6).

The inhibitor of mTOR is a heteroaromatic kinase inhibitor mTOR inhibitor that has a ≥10 fold specificity for mTOR over other PI3K molecules, which shall be referred to as mTOR exclusive kinase inhibitor herein. In certain embodiments, this mTOR exclusive kinase inhibitor is selected from
Torkinib ((2Z)-2-(4-amino-1-propan-2-yl-2H-pyrazolo[3,4-d]pyrimidin-3-ylidene)indol-5-ol; PP242, CAS No. 1092351-67-1),
PP30 (Feldman et al., PLoS Biol., 2009, 7(2):e 1000038 (FIG. 1)),
WAY-600 (4-[6-(1H-indol-5-yl)-1-[1-(pyridin-3-ylmethyl)piperidin-4-yl]pyrazolo[3,4-d]pyrimidin-4-yl]morpholine; CAS No. 1062159-35-6),
WYE-687 (methyl N-[4-[4-morpholin-4-yl-1-[1-(pyridin-3-ylmethyl)piperidin-4-yl]pyrazolo[3,4-d]pyrimidin-6-yl]phenyl]carbamate; CAS No. 1062161-90-3),
PP121 (1-cyclopentyl-3-(1H-pyrrolo[2,3-b]pyridin-5-yl)-1H-pyrazolo[3,4-d]pyrimidin-4-amine; CAS No. 1092788-83-4),
WYE-354 (methyl 4-[6-[4-(methoxycarbonylamino)phenyl]-4-morpholin-4-ylpyrazolo[3,4-d]pyrimidin-1-yl]piperidine-1-carboxylate; CAS No. 1062169-56-5),
KU-0063794 ([5-[2-[(2R,6S)-2,6-dimethylmorpholin-4-yl]-4-morpholin-4-ylpyrido[2,3-d]pyrimidin-7-yl]-2-methoxyphenyl]methanol; CAS No. 938440-64-3) and
AZD8055 ([5-[2,4-bis[(3S)-3-methylmorpholin-4-yl]pyrido[2,3-d]pyrimidin-7-yl]-2-methoxyphenyl]methanol; 1009298-09-2).

The inhibitor of mTOR is a heteroaromatic kinase inhibitor mTOR inhibitor that has a ≥100 fold specificity for mTOR over other PI3K molecules, which shall be referred to as mTOR highly exclusive kinase inhibitor herein. In certain embodiments, this mTOR highly exclusive kinase inhibitor is selected from
OSI-027 (4-[(5Z)-4-amino-5-(7-methoxyindol-2-ylidene)-1H-imidazo[5,1-f][1,2,4]triazin-7-yl]cyclohexane-1-carboxylic acid; CAS No. 936890-98-1),
AZD2014 (3-[2,4-bis[(3S)-3-methylmorpholin-4-yl]pyrido[2,3-d]pyrimidin-7-yl]-N-methylbenzamide; CAS No. 1009298-59-2),
sapanisertib (5-(4-amino-1-propan-2-ylpyrazolo[3,4-d]pyrimidin-3-yl)-1,3-benzoxazol-2-amine MLN0128, INK 128, CAS No. 1224844-38-5),
Torin 1 (1-[4-[4-(1-oxopropyl)-1-piperazinyl]-3-(trifluoromethyl)phenyl]-9-(3-quinolinyl)-benzo[h]-1,6-naphthyridin-2(1H)-one; CAS No. 1222998-36-8),
Torin 2 (9-(6-Amino-3-pyridinyl)-1-[3-(trifluoromethyl)phenyl]-benzo[h]-1,6-naphthyridin-2(1H)-one, CAS No. 1223001-51-1),
GDC-0349 (1-ethyl-3-[4-[4-[(3S)-3-methylmorpholin-4-yl]-7-(oxetan-3-yl)-6,8-dihydro-5H-pyrido[3,4-d]pyrimidin-2-yl]phenyl]urea, CAS No. 1207360-89-1),
XL388 ([7-(6-Aminopyridin-3-yl)-2,3-dihydro-1,4-benzoxazepin-4(5H)-yl][3-fluoro-2-methyl-4-(methylsulfonyl)phenyl]methanone, CAS No. 1251156-08-7),
WYE-125132 (1-[4-[1-(1,4-Dioxaspiro[4.5]decan-8-yl)-4-(8-oxa-3-azabicyclo[3.2.1]octan-3-yl)pyrazolo[3,4-d]pyrimidin-6-yl]phenyl]-3-methylurea;
WYE-132, CAS No. 1144068-46-1),
Palomid 529 (3-(4-methoxybenzyloxy)-8-(1-hydroxyethyl)-2-methoxy-6H-benzo[c]chromen-6-one, P529, CAS No. 914913-88-5),
PF-04691502 (CAS No. 1013101-36-4); GSK1059615 ((Z)-5-((4-(pyridin-4-yl)quinolin-6-yl)methylene)thiazolidine-2,4-dione, CAS No. 958852-01-2),
Gedatolisib (1-(4-((4-(Dimethylamino)piperid in-1-yl)carbonyl)phenyl)-3-(4-(4,6-dimorpholin-4-yl-1,3,5-triazin-2-yl)phenyl)urea,
MHY1485 (4,6-di-4-morpholinyl-N-(4-nitrophenyl)-1,3,5-triazin-2-amine; CAS No. 326914-06-1),
PF-05212384,
PKI-587, CAS No. 1196160-78-3) and
ETP-46464 (2-methyl-2-[4-(2-oxo-9-quinolin-3-yl-4H-[1,3]oxazino[5,4-c]quinolin-1-yl)phenyl]propanenitrile, CAS No. 1345675-02-6).

Rapamycin is the compound (1R,9S,12S,15R,16E,18R,19R,21R,23S,24E,26E,28E,30S,32S,35R)-1,18-dihydroxy-12-{(2R)-1-[(1S,3R,4R)-4-hydroxy-3-methoxycyclohexyl]-2-propanyl}-19,30-dimethoxy-15,17,21,23,29,35-hexamethyl-11,36-dioxa-4-azatricyclo[30.3.1.0-4,9-]hexatriaconta-16,24,26,28-tetraene-2,3,10,14,20-pentone (CAS No. 53123-88-9).

Resveratrol is the compound 5-[(1E)-2-(4-hydroxyphenyl)ethenyl]-1,3-benzenediol (CAS No. 501-36-0).

Brefeldin A is the compound (1R,2E,6S,10E,11aS,13S,14aR)-1,13-Dihydroxy-6-methyl-1,6,7,8,9,11a,12,13,14,14a-decahydro-4H-cyclopenta[t]oxacyclotridecin-4-one (CAS No. 20350-15-6)

Monensin is the compound 4-[2-[5-ethyl-5-[5-[6-hydroxy-6-(hydroxymethyl)-3,5-dimethyl-oxan-2-yl]-3-methyl-oxolan-2-yl]oxolan-2-yl]-9-hydroxy-2,8-dimethyl-1,6-dioxaspiro[4.5]dec-7-yl]-3-methoxy-2-methyl-pentanoic acid (CAS No. 17090-79-8).

Helios is the zinc finger protein encoded by the IKZF2 gene.

Perforin is the protein encoded by the PRF1 gene.

Granzyme B is the protein encoded by the GZMB gene.

KLRG1 is the protein killer cell lectin-like receptor subfamily G member 1 encoded by the KLRG1 gene.

FoxP3 is the protein forkhead box P3 encoded by the FOXP3 gene.

A first aspect of the invention relates to a method for determining a T cell mediated immunity towards a CRISPR associated protein, or towards a homologue of such CRISPR associated protein. The method comprises the steps of
a. providing a cell preparation comprising T cells obtained from a patient;
b. in a stimulation step, contacting said cell preparation with
   an isolated CRISPR associated protein polypeptide, or a homologue thereof, or
   a plurality of peptides, wherein said plurality of peptides represents the amino acid sequence of said CRISPR associated protein polypeptide, or said homologue thereof,
   a cell comprising a CRISPR associated protein or polypeptide, or a homologue thereof,
in the presence of T cells and antigen presenting cells, particularly in the presence of autologous peripheral blood mononuclear cells, under conditions of cell culture, providing activated T cells;
c. optionally, adding an inhibitor of intracellular protein transport, particularly Brefeldin A and/or Monensin, to said cell preparation during the last part of said stimulation step;
d. in a detection step, detecting one or more subpopulations of said activated T cells by
   contacting said activated T cells with a set of molecular probes specific to activated regulatory T cells, and/or
   contacting said activated T cells with a set of molecular probes specific to activated effector T cells, and
providing marked activated regulatory T cells and/or marked activated effector T cells;
e. in a quantification step, determining a number of said marked activated regulatory T cells and a number of said marked activated effector T cells, and optionally, a ratio (T_{REG}/T_{EFF}) of the number of said marked activated regulatory T cells to said marked activated effector T cells.

The method aims to determine if a patient's immune system will react by a cytotoxic immune response upon encounter with a CRISPR associated protein polypeptide, or a homologue thereof, in the context of a CRISPR-mediated therapeutic intervention, particularly an in vivo gene therapy or upon adoptive transfer of gene edited cells using the CRISPR/Cas technology. Due to the high prevalence of S. pyogenes infections, SpCas9 is expected to elicit an adaptive memory immune response in humans. In both in vivo gene therapy and adoptive transfer, the method is suitable for determining the immune response to be expected prior to in vivo gene therapy or prior adoptive transfer of edited cells.

The cell preparation that is used to obtain T cells from the patient will in many embodiments also contain cells from which antigen presenting cells (APC) can be derived. A key feature of this aspect is that the T cells are cultivated together with APC. APC can be derived, inter alia, from the monocyte fraction contained in peripheral blood mononuclear cells (PBMC).

In certain embodiments, the cell preparation is a blood cell preparation, particularly a preparation of PBMC.

Other embodiments include CD3 depleted PBMC and/or autologous polyclonal stimulated T cell lines.

For the detection of Cas specific T_{REG} (activated T_{REG}) and Cas specific T_{EFF} (activated T_{EFF}), a preparation of PBMC encompasses all necessary prerequisites for the stimulation of the cell preparation in the stimulation step such as antigen presenting cells. The cell preparation is maintained in an incubator in cell culture medium containing fetal bovine serum or human antibody.

In certain embodiments, the cell preparation comprises peripheral blood mononuclear cells.

In certain embodiments, the cell preparation comprises T cells and antigen presenting cells.

In certain embodiments, the cell preparation comprises regulatory T cells and/or effector T cells as well as antigen presenting cells.

In certain embodiments, the cell preparation comprises regulatory T cells and effector T cells.

A second aspect of the invention relates to a method for determining a nucleic acid sequence encoding a T cell receptor molecule capable of specifically recognizing an HLA-presented antigen derived from a CRISPR associated protein, particularly a Cas9 or Cas12 protein, or from a homologue of such CRISPR associated protein, comprising the steps of
a. providing a cell preparation comprising T cells obtained from a patient;
b. in a stimulation step, contacting said cell preparation with
   an isolated CRISPR associated protein polypeptide, particularly a Cas9 or Cas12 polypeptide, or a homologue of such CRISPR associated protein, or
   a plurality of peptides, wherein said plurality of peptides represents the amino acid sequence of said CRISPR associated protein polypeptide, particularly said Cas9 or Cas12 polypeptide, or said homologue thereof, or
   a cell comprising a CRISPR associated protein polypeptide, particularly Cas9 or Cas12, or a homologue of such CRISPR associated protein,
in the presence of antigen presenting cells, particularly in the presence of autologous peripheral blood mononuclear cells, providing activated T cells;
c. optionally, adding an inhibitor of intracellular protein transport, particularly Brefeldin A and/or Monensin, to said cell preparation during the last part of said stimulation step;
d. in an isolation step, isolating one or more subpopulations of said activated T cells by
   contacting said activated T cells with a set of molecular probes specific to activated regulatory T cells, and/or
   contacting said activated T cells with a set of molecular probes specific to activated effector T cells,
and removing a population of CRISPR specific regulatory T cells and/or CRISPR specific activated effector T cells from said preparation;
e. in a sequence determination step, determining one or more nucleic acid sequences encoding a CRISPR specific T cell receptor molecule comprised in said population of CRISPR specific T cells.

The method aims to identify and isolate TCR receptor sequences that will enable a patient's immune system to react by a cytotoxic immune response upon encounter with a CRISPR associated protein polypeptide, or a homologue thereof, in the context of a CRISPR-mediated therapeutic intervention, particularly an in vivo gene therapy or upon adoptive transfer of gene edited cells using the CRISPR/Cas technology. Due to the high prevalence of S. pyogenes infections, SpCas9 is expected to elicit an adaptive memory immune response in humans. In both in vivo gene therapy and adoptive transfer, the method is suitable for determining the immune response to be expected prior to in vivo gene therapy or prior adoptive transfer of edited cells.

In particular embodiments, CRISPR specific regulatory T cell receptors are determined, which are expected to be different from CRISPR specific effector T cell receptors.

The cell preparation that is used to obtain CRISPR specific T cell receptors from a patient, will in many embodiments also contain cells from which antigen presenting cells (APC) can be derived.

A key feature of this aspect is that the T cells are cultivated together with APC. APC can be derived, inter alia, from the monocyte fraction contained in peripheral blood mononuclear cells (PBMC).

In certain embodiments of the first of second aspect of the invention, the CRISPR associated protein polypeptide is a Cas9 or Cas12 polypeptide.

In certain embodiments of the first of second aspect of the invention, the CRISPR associated protein polypeptide is a Cas9 polypeptide.

In certain embodiments of the first of second aspect of the invention, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897, A1IQ68 U2UMQ6 or A0Q7Q2 (Uniprot Entry IDs), and having substantially the same biological activity.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897 or A1IQ68 (Uniprot Entry IDs).

In certain embodiments, the cell preparation is contacted in the stimulation step with an isolated CRISPR associated protein polypeptide, or a homologue thereof.

In certain embodiments, the cell preparation is contacted in the stimulation step with an isolated CRISPR associated protein polypeptide.

In certain embodiments, the cell preparation is contacted in the stimulation step with a plurality of peptides, wherein the plurality of peptides represents the amino acid sequence of a CRISPR associated protein polypeptide, or a homologue thereof.

In certain embodiments, the cell preparation is contacted in the stimulation step with a plurality of peptides, wherein the plurality of peptides represents the amino acid sequence of a CRISPR associated protein polypeptide. One peptide typically comprises 8 to 30, particularly 8 to 15, amino acids.

In certain embodiments, the cell preparation is contacted in the stimulation step with an isolated Cas9 or Cas12 polypeptide, particularly with an isolated Cas9 polypeptide, or a homologue thereof.

In certain embodiments, the cell preparation is contacted in the stimulation step with an isolated Cas9 or Cas12 polypeptide, particularly with an isolated Cas9 polypeptide.

In certain embodiments, the cell preparation is contacted in the stimulation step with a plurality of peptides, wherein the plurality of peptides represents the amino acid sequence of a Cas9 or Cas12 polypeptide, particularly with an isolated Cas9 polypeptide, or a homologue thereof.

In certain embodiments, the cell preparation is contacted in the stimulation step with a plurality of peptides, wherein the plurality of peptides represents the amino acid sequence of a Cas9 or Cas12 polypeptide, particularly with an isolated Cas9 polypeptide.

In certain embodiments of the first or second aspect of the invention, the cell preparation is contacted in the stimulation step with a cell comprising a CRISPR associated protein or polypeptide. This cell may have been subject to gene therapy method by which its genome was edited using a CRISPR mediated technology employing a potentially immunogenic CRISPR associated protein or polypeptide. For genome editing, the CRISPR associated protein polypeptide may be provided by cellular uptake of said polypeptide or by cellular uptake of a polynucleotide sequence encoding a CRISPR associated protein polypeptide and subsequent expression of said polypeptide. In this case, the stimulation step serves to assay the immunogenicity of the cell before a known background of immune response in the patient.

In certain embodiments, the cell preparation is contacted in the stimulation step with a cell comprising a nucleic acid encoding the CRISPR associated protein or polypeptide. The rationale given in the preceding paragraph applies mutatis mutandis.

In certain embodiments, the isolated Cas9 polypeptide or plurality of peptides used in the stimulation step is derived from Streptococcus pyogenes.

The stimulation step is performed under conditions of cell culture.

In certain embodiments, the contacting in the stimulation step is performed between 2 h and 25 h, in particular 12 h to 20 h, more particularly 12 h to 18 h.

If a plurality of peptides is used in the stimulation step, one peptide typically comprises 8 to 30, particularly 8 to 15, amino acids. The plurality of peptides may be produced as described in EP1051619 A2 or EP1257290 A2.

In the stimulation step of the first or second aspect of the invention, the polypeptide or peptide concentration is adjusted to the number of PBMC in such a way that a sufficient T cell response is achieved. Typically, a concentration of 1 to 50 μg/ml, particularly 4 to 10 μg/ml, polypeptide or a concentration of 0.1 to 10 μg/ml, particularly 1 μg/ml, peptide in a peptide pool is used for up to 10*10⁶ PBMC.

In certain embodiments of the first or second aspect of the invention, antigen presenting cells, in particular monocyte-derived dendritic cells and/or B cells are present in the stimulation step. The antigen presenting cells present fragments of the CRISPR associated protein polypeptide, or a homologue thereof. The antigen presenting cell may also present those peptides that can be presented by their respective HLA (human leukocyte antigen) set, of the entire plurality of peptides. The plurality of peptides represents the amino acid sequence of said CRISPR associated protein polypeptide, or said homologue thereof, or may present fragments thereof.

The antigen presenting cell may be incubated with the CRISPR associated protein polypeptide, or a homologue thereof, or the plurality of peptides, wherein said plurality of peptides represents the amino acid sequence of said CRISPR associated protein polypeptide, or said homologue thereof, prior to the stimulation step.

In certain embodiments of the first or second aspect of the invention, an inhibitor of intracellular protein transport is present during the last part of the stimulation step, particularly an inhibitor targeting the Golgi apparatus and/or an inhibitor of vesicular transport. The addition of an inhibitor of intracellular protein transport allows intracellular detection of proteins that would be secreted if said inhibitor were not present. Adding this inhibitor is important for the correct assessment of effector molecules, for example TNFalpha or IFNgamma.

In certain embodiments of the first or second aspect of the invention, the inhibitor of intracellular protein transport is Brefeldin A, a Brefeldin A analogue, a Brefeldin A derivative, Monensin, a Monensin analogue or a Monensin derivative.

In certain embodiments, the inhibitor of intracellular protein transport is Brefeldin A or Monensin.

In certain embodiments, the inhibitor of intracellular protein transport is Brefeldin A and Monensin.

This means, both inhibitors are added simultaneously.

In certain embodiments, the inhibitor of intracellular protein transport is present during the last part of the stimulation step wherein the last part is defined as the time starting 1 h to 10 h, in particular 1 h to 5 or 6 h, after the beginning of the stimulation step.

If an isolated CRISPR associated protein polypeptide or homologue thereof is used in the stimulation step, the inhibitor of intracellular protein transport is added at a time point between 2 and 10 h after the beginning of the stimulation step.

If an isolated CRISPR associated protein polypeptide or homologue thereof is used in the stimulation step, the inhibitor of intracellular protein transport is added at a time point between 2 and 6 h after the beginning of the stimulation step.

If an isolated CRISPR associated protein polypeptide or homologue thereof is used in the stimulation step, the inhibitor of intracellular protein transport is added at the time point 6 h after the beginning of the stimulation step.

If a plurality of peptides representing the amino acid sequence of a CRISPR associated protein polypeptide, or a homologue thereof, is used in the stimulation step, the inhibitor of intracellular protein transport is added at a time point between 1 and 10 h after the beginning of the stimulation step.

If a plurality of peptides representing the amino acid sequence of a CRISPR associated protein polypeptide, or a homologue thereof, is used in the stimulation step, the inhibitor of intracellular protein transport is added at a time point between 1 and 5 h after the beginning of the stimulation step.

If a plurality of peptides representing the amino acid sequence of a CRISPR associated protein polypeptide, or a homologue thereof, is used in the stimulation step, the inhibitor of intracellular protein transport is added at the time point 1 h after the beginning of the stimulation step.

If an edited cell is used in the stimulation step, the inhibitor of intracellular protein transport is added at a time point between 1 and 24 h after the beginning of the stimulation step.

In certain embodiments, the inhibitor of intracellular protein transport is present for 2 to 12 h, in particular 6 to 10 h, in the stimulation step.

Other alternative embodiments employ methods of detecting Cas9 reactive T cells by surface marker expression (CD40L/CD137/CD69 and others) only; these embodiments do not require golgi inhibition but addition of e.g. aCD40 antibody during stimulation. A synonymous term for CD154 is CD40L.

CD154 (CD40L) is a ligand that can interact with the receptor CD40. CD40 is expressed for example on thrombocytes. Thus, in an unstimulated cell preparation with various cell types, cells expressing CD40 may be present. If such cell preparation is stimulated as described above, Cas9 reactive T_{EFF} cells respond by the expression of activation specific marker molecules such as CD154. To prevent binding of CD154⁺ T_{EFF} cells to CD40⁺ cells, CD40 may be blocked by a ligand such as an anti-CD40 antibody. This allows detection and/or isolation of CD154⁺ T_{EFF} cells by using a ligand specific to CD154.

In the detection step of the first aspect of the invention, one or more subpopulations of activated T cells from the stimulated cell preparation are detected. In particular, activated regulatory T cells and/or activated effector T cells are detected.

In the isolation step of the second aspect of the invention, one or more subpopulations of activated T cells from the stimulated cell preparation are isolated for TCR sequence determination, e.g. by PCR or high throughput sequencing methods. In particular, activated regulatory T cells and/or activated effector T cells are isolated for sequence elucidation.

In certain embodiments of the first or second aspect of the invention, the set of molecular probes specific to activated regulatory T cells comprises ligands specific to
CD3, CD4, CD137, CD154 and CD25, and
one or more ligands specific to any one of FoxP3, helios and CD127,
and optionally, one or more ligands specific to any one of CD69, CD71, CD103, CD134, GARP, HLA-DR, IFNγ, IL-10, KLRG1, LAP, SATB1, TGFβ or TNFα.

In certain embodiments, the set of molecular probes specific to activated regulatory T cells comprises ligands specific to CD3, CD4, CD137, CD154, CD25, FoxP3, helios and CD127 and optionally, one or two ligands specific to any one of CD69, CD71, CD103, CD134, GARP, HLA-DR, IFNγ, IL-10, KLRG1, LAP, SATB1, TGFβ or TNFα. In certain embodiments, the set of molecular probes specific to activated regulatory T cells comprises ligands specific to CD3, CD4, CD137, CD154, CD25 and FoxP3 and CD127 and optionally, one or two ligands specific to any one of CD69, CD71, CD103, CD134, GARP, HLA-DR, IFNγ, IL-10, KLRG1, LAP, SATB1, TGFβ or TNFα. In certain embodiments, the set of molecular probes specific to activated regulatory T cells comprises ligands specific to CD3, CD4, CD137, CD154, CD25 and FoxP3 and optionally, one or two ligands specific to any one of CD69, CD71, CD103, CD134, GARP, HLA-DR, IFNγ, IL-10, KLRG1, LAP, SATB1, TGFβ or TNFα. In certain embodiments, the set of molecular probes specific to activated regulatory T cells comprises ligands specific to CD3, CD4, CD137, CD154, CD25 and helios and optionally, one or two ligands specific to any one of CD69, CD71, CD103, CD134, GARP, HLA-DR, IFNγ, IL-10, KLRG1, LAP, SATB1, TGFβ or TNFα. In certain embodiments, the set of molecular probes specific to activated regulatory T cells comprises ligands specific to CD3, CD4, CD137, CD154, CD25 and CD127 and optionally, one or two ligands specific to any one of CD69, CD71, CD103, CD134, GARP, HLA-DR, IFNγ, IL-10, KLRG1, LAP, SATB1, TGFβ or TNFα. In certain embodiments, the set of molecular probes specific to activated regulatory T cells comprises ligands specific to CD3, CD4, CD137, CD154, CD25, FoxP3, helios and CD127.

In certain embodiments, the set of molecular probes specific to activated regulatory T cells comprises ligands specific to CD3, CD4, CD137, CD154, CD25 and FoxP3 and CD127.

In certain embodiments, the set of molecular probes specific to activated regulatory T cells comprises ligands specific to CD3, CD4, CD137, CD154, CD25 and FoxP3.

In certain embodiments, the set of molecular probes specific to activated regulatory T cells comprises ligands specific to CD3, CD4, CD137, CD154, CD25 and helios.

In certain embodiments, the set of molecular probes specific to activated regulatory T cells comprises ligands specific to CD3, CD4, CD137, CD154, CD25 and CD127.

In certain embodiments of the first or second aspect of the invention, a cell is assigned an activated regulatory T cell that is
positive for CD3, CD4, CD137 and CD25, wherein CD25 is highly expressed, and negative for CD154, and
positive for FoxP3 and/or positive helios and/or negative for CD127,
   and optionally, positive for any one of CD69, CD71, CD103, CD134, GARP, HLA-DR, IFNγ, IL-10, KLRG1, LAP, SATB1, TGFβ or TNFα. In certain embodiments, a cell is assigned an activated regulatory T cell that is
positive for CD3, CD4, CD137 and CD25, wherein CD25 is highly expressed, and negative for CD154, and
positive for FoxP3 and positive helios and negative for CD127,
   and optionally, positive for any one of CD69, CD71, CD103, CD134, GARP, HLA-DR, IFNγ, IL-10, KLRG1, LAP, SATB1, TGFβ or TNFα. In certain embodiments, a cell is assigned an activated regulatory T cell that is
positive for CD3, CD4, CD137 and CD25, wherein CD25 is highly expressed, and negative for CD154, and
positive for FoxP3 and negative for CD127,
   and optionally, positive for any one of CD69, CD71, CD103, CD134, GARP, HLA-DR, IFNγ, IL-10, KLRG1, LAP, SATB1, TGFβ or TNFα.

In certain embodiments, a cell is assigned an activated regulatory T cell that is
positive for CD3, CD4, CD137 and CD25, wherein CD25 is highly expressed, and negative for CD154, and
positive for FoxP3,
   and optionally, positive for any one of CD69, CD71, CD103, CD134, GARP, HLA-DR, IFNγ, IL-10, KLRG1, LAP, SATB1, TGFβ or TNFα. In certain embodiments, a cell is assigned an activated regulatory T cell that is
positive for CD3, CD4, CD137 and CD25, wherein CD25 is highly expressed, and negative for CD154, and
negative for CD127,
   and optionally, positive for any one of CD69, CD71, CD103, CD134, GARP, HLA-DR, IFNγ, IL-10, KLRG1, LAP, SATB1, TGFβ or TNFα. In certain embodiments, a cell is assigned an activated regulatory T cell that is
positive for CD3, CD4, CD137 and CD25, wherein CD25 is highly expressed, and negative for CD154, and
positive for FoxP3 and positive helios and negative for CD127.

In certain embodiments, a cell is assigned an activated regulatory T cell that is
positive for CD3, CD4, CD137 and CD25, wherein CD25 is highly expressed, and negative for CD154, and
positive for FoxP3 and negative for CD127.

In certain embodiments, a cell is assigned an activated regulatory T cell that is
positive for CD3, CD4, CD137 and CD25, wherein CD25 is highly expressed, and negative for CD154, and
positive for FoxP3.

In certain embodiments, a cell is assigned an activated regulatory T cell that is
positive for CD3, CD4, CD137 and CD25, wherein CD25 is highly expressed, and negative for CD154, and
negative for CD127.

In certain embodiments of the first or second aspect of the invention, the set of molecular probes specific to activated effector T cells comprises
a. ligands specific to CD3, CD4, CD137 and CD154, and optionally, one or more ligands specific to any one of CD69, CD71, CD80, CD86, CD107a, CD134, Granzyme B, HLA-DR, IFNγ, IL-2, KLRG1, Perforin or TNFα; and/or
b. ligands specific to CD3, CD8 and CD137, and optionally, one or more ligands specific to any one of CD69, CD71, CD80, CD86, CD107a, CD134, Granzyme B, HLA-DR, IFNγ, IL-2, KLRG1, Perforin or TNFα; and/or
c. ligands specific to CD3, CD4 and CD137 and one or more ligands specific to CD25, FoxP3 and helios, and optionally, one or more ligands specific to any one of CD69, CD71, CD80, CD86, CD107a, CD134, Granzyme B, HLA-DR, IFNγ, IL-2, KLRG1, Perforin or TNFα.

The ligands listed under item (a) are suitable for the detection of a subpopulation of activated effector T cells. This subpopulation is described in the section “terms and definitions” as cells showing the expression profile (c). The ligands listed under item (b) are suitable for the detection of the subpopulation of activated effector T cells showing the expression profile (d) as described in the section “terms and definitions”. The ligands listed under item (c) are suitable for the detection of the subpopulation of activated effector T cells showing the expression profile (e) as described in the section “terms and definitions”. In the detection step, only one of the subpopulations described above, two of said subpopulations or all subpopulations may be determined.

In certain embodiments, the set of molecular probes specific to activated effector T cells comprises
a. ligands specific to CD3, CD4, CD137 and CD154, and optionally, one or two ligands specific to any one of CD69, CD71, CD80, CD86, CD107a, CD134, Granzyme B, HLA-DR, IFNγ, IL-2, KLRG1, Perforin or TNFα; and/or
b. ligands specific to CD3, CD8 and CD137, and optionally, one or two ligands specific to any one of CD69, CD71, CD80, CD86, CD107a, CD134, Granzyme B, HLA-DR, IFNγ, IL-2, KLRG1, Perforin or TNFα; and/or
c. ligands specific to CD3, CD4 and CD137 and one or two ligands specific to CD25, FoxP3 and helios, and optionally, one or two ligands specific to any one of CD69, CD71, CD80, CD86, CD107a, CD134, Granzyme B, HLA-DR, IFNγ, IL-2, KLRG1, Perforin or TNFα.

In certain embodiments, the set of molecular probes specific to activated effector T cells comprises
a. ligands specific to CD3, CD4, CD137 and CD154; and/or
b. ligands specific to CD3, CD8 and CD137; and/or
c. ligands specific to CD3, CD4 and CD137 and one or more ligands specific to CD25, FoxP3 and helios.

In certain embodiments, the set of molecular probes specific to activated effector T cells comprises
a. ligands specific to CD3, CD4, CD137 and CD154; and
b. ligands specific to CD3, CD8 and CD137; and
c. ligands specific to CD3, CD4 and CD137 and one or more ligands specific to CD25, FoxP3 and helios.

In certain embodiments, the set of molecular probes specific to activated effector T cells comprises ligands specific to CD3, CD4, CD137 and CD154.

In certain embodiments, the set of molecular probes specific to activated effector T cells comprises ligands specific to CD3, CD8 and CD137.

In certain embodiments, the set of molecular probes specific to activated effector T cells comprises ligands specific to CD3, CD4 and CD137 and one or more ligands specific to CD25, FoxP3 and helios.

In certain embodiments, the set of molecular probes specific to activated effector T cells comprises ligands specific to CD3, CD4, CD137 and CD25.

In certain embodiments, the set of molecular probes specific to activated effector T cells comprises ligands specific to CD3, CD4, CD137 and FoxP3.

In certain embodiments of the first or second aspect of the invention, a cell is assigned an activated effector T cell that is positive for CD3 and CD137, and
a. positive for CD4 and CD154 or
b. positive for CD8 or
c. positive for CD4 and
   positive for CD25, wherein CD25 is lowly expressed, and/or
   negative for FoxP3 or helios;
      and optionally, positive for any one of CD69, CD71, CD80, CD86, CD107a, CD134, Granzyme B, HLA-DR, IFNγ, IL-2, KLRG1, Perforin or TNFα.

The expression of marker molecules listed here under item (a), (b) or (c) may be detected by using the ligands described under items (a), (b) or (c) above.

In certain embodiments, a cell is assigned an activated effector T cell that is positive for CD3, CD137, CD4 and CD154.

In certain embodiments, a cell is assigned an activated effector T cell that is positive for CD3,

CD137 and CD8.

In certain embodiments, a cell is assigned an activated effector T cell that is positive for CD3, CD137 and CD25, wherein CD25 is lowly expressed and negative for FoxP3 and helios.

In certain embodiments, a cell is assigned an activated effector T cell that is positive for CD3, CD137 and CD25, wherein CD25 is lowly expressed and negative for FoxP3.

In certain embodiments, a cell is assigned an activated effector T cell that is positive for CD3, CD137 and CD25, wherein CD25 is lowly expressed.

In certain embodiments, a cell is assigned an activated effector T cell that is positive for CD3, CD137 and negative for FoxP3.

The activation marker CD69, CD71, CD80, CD86, CD107a, CD134, Granzyme B, HLA-DR, IFNγ,

IL-2, KLRG1, Perforin or TNFα may be detected in addition to the marker molecules described above. The activation markers are expressed on activated T cells.

In certain embodiments, one or more molecular probes are added in the stimulation step and/or the detection step.

In certain embodiments, one or more molecular probes are added in the detection step

In certain embodiments of the first or second aspect of the invention, the ligand is an antibody, an antibody fragment or antibody-like molecule.

In certain embodiments, the ligand is an antibody or antibody fragment.

In certain embodiments, the ligand is an antibody.

Surface molecule expression may also be assessed by adding detection antibodies to stimulation cultures e.g.: CD154 and CD137. In case CD154 is used, CD154 detection antibody may be added to culture at stimulation initiation or after stimulation. In the latter case, an antibody against CD40 may be added to facilitate CD154 detection.

In certain embodiments, the molecular probe, in particular the antibody, is conjugated to a detectable marker.

In certain embodiments, the molecular probe, in particular the antibody, is conjugated to a particle, bead, dye or enzyme.

In certain embodiments, the molecular probe, in particular the antibody, is conjugated to a fluorescent dye.

The molecular probe may be analyzed by ELISPOT, ELISA, multiplex assays, flow cytometry (e.g. FACS) or fluorescence microscopy.

In certain embodiments, a ratio of the number of marked activated regulatory T cells to the number of marked activated effector T cells (T_{REG}/T_{EFF}) is calculated and the ratio is assigned to a probability of the patient reacting to a therapeutic comprising a CRISPR associated protein, or towards a therapeutic comprising a homologue thereof, by a cytotoxic immune response.

In certain embodiments of the first aspect of the invention, a ratio of the number of marked activated regulatory T cells to the number of marked activated effector T cells (T_{REG}/T_{EFF}) is calculated and the ratio is assigned to a probability of the patient reacting to a therapeutic comprising a Cas9 or Cas12 polypeptide, particularly a Cas 9 polypeptide, or towards therapeutic comprising a homologue of said Cas9 or Cas12 polypeptide, by a cytotoxic immune response.

In certain embodiments of the first aspect of the invention, a ratio T_{REG}/T_{EFF}<0.5 is assigned to a high risk, a ratio 0.5≤T_{REG}/T_{EFF}<1 is assigned to a medium risk and a ratio T_{REG}/T_{EFF}≥1 is assigned to a low risk of said patient reacting to Cas9, or towards a homologue thereof, by a cytotoxic immune response.

In certain embodiments, a ratio T_{REG}/T_{EFF}<0.5 is assigned to a high risk, a ratio 0.5≤T_{REG}/T_{EFF}<1 is assigned to a medium risk and a ratio T_{REG}/T_{EFF}≥1 is assigned to a low risk of said patient reacting to Cas9, or towards a homologue thereof, by a cytotoxic immune response, wherein the T_{EFF} cells are CD4⁺ and CD8⁻.

In certain embodiments of the second aspect of the invention, the expression of MHC-II isotypes is determined for said patient, and said and said sequences encoding a CRISPR specific T cell receptor molecule are assigned to an MHC-II isotype group.

In certain embodiments of the second aspect of the invention, only CRISPR specific regulatory T cells are isolated, and nucleic acids encoding CRISPR specific regulatory T cell receptor molecules are determined.

In certain embodiments of the second aspect of the invention, only CRISPR specific effector T cells are isolated, and nucleic acids encoding CRISPR specific effector T cell receptor molecules are determined.

A third aspect of the invention relates to a method for preparing a preparation of T cells specifically reactive towards a CRISPR associated protein, particularly Cas9 or Cas12, or towards a homologue thereof, comprising the steps of
providing a T cell preparation; wherein in particular said T cell preparation is a preparation of regulatory T cells
introducing a nucleic acid expression construct into said T cell preparation, yielding a transgene T cell preparation,
   wherein said nucleic acid expression construct encodes a T cell receptor molecule capable of specifically recognizing an HLA-presented antigen derived from a CRISPR associated protein, particularly a Cas9 or Cas12 protein, or from a homologue of such CRISPR associated protein.

A fourth aspect of the invention relates to a method for preparing a preparation of regulatory T cells specifically reactive towards a CRISPR associated protein, particularly Cas9 or Cas12, or towards a homologue of such CRISPR associated protein. The method comprises the steps of
a. providing a cell preparation comprising T cells;
b. in a first isolation step, isolating regulatory T cells using a set of molecular probes specific to regulatory T cells,
c. in a stimulation step, contacting said cell preparation with
   an isolated CRISPR associated protein polypeptide, or a homologue thereof, or
   a plurality of peptides, wherein said plurality of peptides represents the amino acid sequence of said CRISPR associated protein polypeptide, or said homologue thereof,
providing activated T cells in a stimulated cell preparation;
d. in a second isolation step, isolating activated regulatory T cells from said stimulated cell preparation using
   a set of molecular probes specific to activated regulatory T cells, or
   a set of molecular probes specific to activation-specific marker molecules;
e. in a cell proliferation step, cultivating said activated regulatory T cells,
wherein either one of the first and second isolation step is performed or both first and second isolation steps are performed.

In certain embodiments, the method comprises the steps of
a. providing a cell preparation comprising T cells;
b. in a first isolation step, isolating regulatory T cells using a set of molecular probes specific to regulatory T cells,
c. in a stimulation step, contacting said cell preparation with
   an isolated CRISPR associated protein polypeptide, or a homologue thereof, or
   a plurality of peptides, wherein said plurality of peptides represents the amino acid sequence of said CRISPR associated protein polypeptide, or said homologue thereof,
e. in a cell proliferation step, cultivating said activated regulatory T cells.

In certain embodiments, the method comprises the steps of
a. providing a cell preparation comprising T cells;
c. in a stimulation step, contacting said cell preparation with
   an isolated CRISPR associated protein polypeptide, or a homologue thereof, or
   a plurality of peptides, wherein said plurality of peptides represents the amino acid sequence of said CRISPR associated protein polypeptide, or said homologue thereof,
providing activated T cells in a stimulated cell preparation;
d. in a second isolation step, isolating activated regulatory T cells from said stimulated cell preparation using
   a set of molecular probes specific to activated regulatory T cells, or
   a set of molecular probes specific to activation-specific marker molecules;
e. in a cell proliferation step, cultivating said activated regulatory T cells.

In certain embodiments, the method comprises the steps of
a. providing a cell preparation comprising T cells;
b. in a first isolation step, isolating regulatory T cells using a set of molecular probes specific to regulatory T cells,
c. in a stimulation step, contacting said cell preparation with
   an isolated CRISPR associated protein polypeptide, or a homologue thereof, or
   a plurality of peptides, wherein said plurality of peptides represents the amino acid sequence of said CRISPR associated protein polypeptide, or said homologue thereof,
providing activated T cells in a stimulated cell preparation;
d. in a second isolation step, isolating activated regulatory T cells from said stimulated cell preparation using
   a set of molecular probes specific to activated regulatory T cells, or
   a set of molecular probes specific to activation-specific marker molecules;
e. in a cell proliferation step, cultivating said activated regulatory T cells.

The method according to the third or fourth aspect of the invention aims to provide a preparation of regulatory T cells specifically reactive towards a CRISPR associated protein, or towards a homologue thereof. This means, that the regulatory T cell may react towards a CRISPR associated protein in concert with other components of T cell immunity such as antigen presentation by antigen presenting (APC) cells. The APCs present fragments of the CRISPR associated protein polypeptide via HLA molecules. Upon interaction between HLA molecules of the APC cell and the T cell receptor of the regulatory T cell, the regulatory T cell is subsequently activated by downstream signalling pathways.

In certain embodiments, the cell preparation is a blood cell preparation.

In certain embodiments, the cell preparation comprises peripheral blood mononuclear cells.

In certain embodiments, the cell preparation comprises T cells and antigen presenting cells.

In certain embodiments, the cell preparation comprises regulatory T cells and/or effector T cells as well as antigen presenting cells.

In certain embodiments, the cell preparation comprises regulatory T cells and effector T cells.

In certain embodiments, the CRISPR associated protein polypeptide is a Cas9 or Cas12 polypeptide.

In certain embodiments, the CRISPR associated protein polypeptide is a Cas9 polypeptide.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897, A1IQ68 U2UMQ6 or A0Q7Q2 (Uniprot Entry IDs), and having substantially the same biological activity.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897 or A1IQ68 (Uniprot Entry IDs).

In certain embodiments, the cell preparation is contacted in the stimulation step with an isolated CRISPR associated protein polypeptide, or a homologue thereof.

In certain embodiments, the cell preparation is contacted in the stimulation step with an isolated CRISPR associated protein polypeptide.

In certain embodiments, the cell preparation is contacted in the stimulation step with a plurality of peptides, wherein the plurality of peptides represents the amino acid sequence of a CRISPR associated protein polypeptide, or a homologue thereof.

In certain embodiments, the cell preparation is contacted in the stimulation step with a plurality of peptides, wherein the plurality of peptides represents the amino acid sequence of a CRISPR associated protein polypeptide.

In certain embodiments, the cell preparation is contacted in the stimulation step with an isolated Cas9 or Cas12 polypeptide, particularly with an isolated Cas9 polypeptide, or a homologue thereof.

In certain embodiments, the cell preparation is contacted in the stimulation step with an isolated

Cas9 or Cas12 polypeptide, particularly with an isolated Cas9 polypeptide.

In certain embodiments, the cell preparation is contacted in the stimulation step with a plurality of peptides, wherein the plurality of peptides represents the amino acid sequence of a Cas9 or Cas12 polypeptide, particularly with an isolated Cas9 polypeptide, or a homologue thereof.

In certain embodiments, the cell preparation is contacted in the stimulation step with a plurality of peptides, wherein the plurality of peptides represents the amino acid sequence of a Cas9 or Cas12 polypeptide, particularly with an isolated Cas9 polypeptide.

In certain embodiments, the isolated Cas9 polypeptide or plurality of peptides used in the stimulation step is derived from Streptococcus pyogenes.

In certain embodiments, the cell preparation is contacted in the stimulation step with a cell comprising a CRISPR associated protein or polypeptide. This cell may have been subject to gene therapy method by which its genome was edited using a CRISPR mediated technology employing a potentially immunogenic CRISPR associated protein or polypeptide. For genome editing, the CRISPR associated protein polypeptide may be provided by cellular uptake of said polypeptide or by cellular uptake of a polynucleotide sequence encoding a CRISPR associated protein polypeptide and subsequent expression of said polypeptide.

In certain embodiments, the cell preparation in the stimulation step is a cell culture.

In certain embodiments, the contacting in the stimulation step is performed between 2 h and 24 h, in particular 12 h to 20 h, more particularly 12 h to 18 h.

If a plurality of peptides is used in the stimulation step, one peptide typically comprises 8 to 30, particularly 8 to 15, amino acids. The plurality of peptides may be produced as described in EP1051619 A2 or EP1257290 A2 or the corresponding US documents U.S. Pat. No. 8,932,806 (B1) or US2004106159 (A1) which are incorporated herein by reference.

In the stimulation step, the polypeptide or peptide concentration is adjusted to the number of PBMC in such a way that a sufficient T cell response is achieved. Typically, a concentration of 1 μg/ml peptide is used for up to 10*10⁶ PBMC.

In certain embodiments of the fourth aspect of the invention, antigen presenting cells, in particular monocyte-derived dendritic cells and/or B cells are present in the stimulation step. The antigen presenting cells present fragments of the CRISPR associated protein polypeptide, or a homologue thereof. The antigen presenting cell may also present the plurality of peptides, wherein said plurality of peptides represents the amino acid sequence of said CRISPR associated protein polypeptide, or said homologue thereof, or may present fragments thereof.

The antigen presenting cell may be incubated with the CRISPR associated protein polypeptide, or a homologue thereof, or the plurality of peptides, wherein said plurality of peptides represents the amino acid sequence of said CRISPR associated protein polypeptide, or said homologue thereof, prior to the stimulation step.

The method of the fourth aspect of the invention may either start with the isolation of regulatory T cells (first isolation step) followed by the stimulation step and the proliferation step. Optionally, the second isolation step may be performed before proliferation. Typically, the set of molecular probes specific to activation-specific marker molecules is used in this case.

Alternatively, the cell preparation comprising T cells may be first stimulated followed by an isolation step (second isolation step) and the proliferation step. If only the second isolation step is performed, the set of molecular probes specific to activated regulatory T cells is used.

In the isolation steps, cells are isolated by using molecular probes that target molecules expressed on the surface of said cells. The isolation is performed by negative and/or positive selection. The isolation steps described herein may be further divided into several substeps, for example one isolation step may comprise a negative selection step followed by two positive selection steps.

In certain embodiment of the third aspect of the invention, the T cell preparation is isolated using a set of molecular probes specific to non-activated regulatory T-cells.

In certain embodiments of the third or fourth aspect of the invention, the set of molecular probes specific to non-activated regulatory T cells comprises ligands specific to CD3, CD4, CD25 and CD127 and/or CD137. This set of molecular probes may be used in the first isolation step.

In certain embodiments, the set of molecular probes specific to non-activated regulatory T cells comprises ligands specific to CD3, CD4, CD25 and CD127.

In certain embodiments, the set of molecular probes specific to non-activated regulatory T cells comprises ligands specific to CD3, CD4, CD25 and CD137.

In certain embodiments, the set of molecular probes specific to non-activated regulatory T cells comprises ligands specific to CD3, CD4, CD25, CD127 and CD137.

In certain embodiments, the ligands specific to CD3, CD4 or CD25 are used for positive selection, wherein in case of a ligand specific to CD25 only cells with a high CD25 expression are selected, and wherein said ligands specific to CD127 and CD137 are used for negative selection in the first isolation step.

In certain embodiments, the set of molecular probes specific to activated regulatory T cells comprises ligands specific to CD3, CD4, CD137, CD154, CD25 and CD127, and optionally, one or more ligands specific to CD69, CD71, CD103, CD134, GARP, HLA-DR, KLRG1 or LAP.

In certain embodiments, the set of molecular probes specific to activated regulatory T cells comprises ligands specific to CD3, CD4, CD137, CD154, CD25 and CD127, and optionally, one or two ligands specific to CD69, CD71, CD103, CD134, GARP, HLA-DR, KLRG1 or LAP.

In certain embodiments, the set of molecular probes specific to activated regulatory T cells comprises ligands specific to CD3, CD4, CD137, CD154, CD25 and CD127.

The set of molecular probes specific to activated regulatory T cells may be used in the second isolation step according to the fourth aspect of the invention.

In certain embodiments, the set of molecular probes specific to activation-specific marker molecules comprises a ligand specific to CD137 and optionally one or more ligands specific to CD69, CD71, CD103, CD134, GARP, HLA-DR, KLRG1 or LAP. This set may be used in the second isolation step.

In certain embodiments, the set of molecular probes specific to activation-specific marker molecules comprises a ligand specific to CD137 and optionally one or two ligands specific to

CD69, CD71, CD103, CD134, GARP, HLA-DR, KLRG1 or LAP. In certain embodiments, the set of molecular probes specific to activation-specific marker molecules comprises a ligand specific to CD137.

In certain embodiments, the ligands used in the second isolation step according to the fourth aspect may be used for positive or negative selection as follows:

The ligands specific to CD3, CD4, CD25, CD137, CD69, CD71, CD103, CD134, GARP, HLA-DR, KLRG1 or LAP may be used for positive selection, wherein in case of a ligand specific to CD25 only cells with a high CD25 expression are selected.

The ligands specific to CD127 or CD154 may be used for negative selection.

For example, regulatory T cells may be isolated in the first isolation step by positive selection using ligands specific to CD3, CD4 and CD25 followed by negative selection using ligands specific to CD127 and CD137. The regulatory T cells are subsequently stimulated and before the proliferation step, activated regulatory T cells are positively selected by using a ligand specific to CD137 (second isolation step).

For example, regulatory T cells may be isolated in the first isolation step by positive selection using ligands specific to CD3, CD4 and CD25 followed by negative selection using ligands specific to CD127. The regulatory T cells are subsequently stimulated and before the proliferation step, activated regulatory T cells are positively selected by using a ligand specific to CD137 (second isolation step).

An alternative strategy might be the stimulation of the T cell preparation and performing only the second isolation step by using ligands specific to CD3, CD4, CD25 and CD137 for positive selection and using ligands specific to CD127 and CD154 for negative selection.

In certain embodiments, one or more molecular probes are added in the stimulation step and/or the isolation step.

In certain embodiments, one or more molecular probes are added in the isolation step.

Surface molecule expression may also be assessed by adding detection antibodies to stimulation cultures e.g.: CD154 and CD137. In case CD154 is used, CD154 detection antibody may be added to culture at stimulation initiation or after stimulation. In the latter case, an antibody against CD40 may be added to facilitate CD154 detection.

In certain embodiments, the molecular probe, in particular the antibody, is conjugated to a detectable marker.

In certain embodiments, the molecular probe, in particular the antibody, is conjugated to a particle, bead, dye or enzyme.

In certain embodiments, the molecular probe, in particular the antibody, is conjugated to a fluorescent dye.

The selection of a detectable marker of the molecular probe depends on the method chosen for the isolation of activated regulatory T cells. For example, the activated regulatory T cells may be marked with suitable antibodies conjugated to a fluorescent dye and isolated using FACS. Antibodies conjugated to magnetic beads may be used for magnetic cell separation. Further methods for the isolation of activated regulatory T cells are described in Scheonbrunn et al. 2012, J Immunol 189(12):5985-5994 and Bacher and Scheffold 2013, Cytometry, 83A: 692-701, DOI: 10.1002/cyto.a.22317,

In certain embodiments according to the third or fourth aspect of the invention, a transgene T cell preparation is kept under conditions of cell culture in a cell proliferation step.

In certain embodiments of the third or fourth aspect of the invention, IL-2 is present in the cell proliferation step.

In certain embodiments of the third or fourth aspect of the invention, IL-2 and optionally any one of resveratrol, a resveratrol analogue, a resveratrol derivative, or an mTor inhibitor are present in the cell proliferation step.

In certain embodiments, IL-2 and any one of resveratrol, a resveratrol analogue or a resveratrol derivative, or IL-2 and any one of rapamycin, a rapamycin analogue or a rapamycin derivative are present in the cell proliferation step.

In certain embodiments, IL-2 and resveratrol, or IL-2 and rapamycin are present in the cell proliferation step.

In certain embodiments, IL-2 and any one of rapamycin, a rapamycin analogue or a rapamycin derivative are present in the cell proliferation step.

In certain embodiments, IL-2 and rapamycin are present in the cell proliferation step.

In certain embodiments, 50 IU/ml to 5000 IU/ml of IL-2 are present in said cell proliferation step and optionally 50 nM to 150 nM resveratrol, a resveratrol analogue, a resveratrol derivative or mTor inhibitor (particularly rapamycin) are present in said cell proliferation step.

In certain embodiments, 50 IU/ml to 2000 IU/ml of IL-2 are present in said cell proliferation step and optionally 50 nM to 150 nM resveratrol, a resveratrol analogue, a resveratrol derivative or mTor inhibitor (particularly rapamycin) are present in said cell proliferation step.

In certain embodiments, 200 IU/ml to 1000 IU/ml of IL-2 are present in said cell proliferation step and optionally 100 nM resveratrol, a resveratrol analogue, a resveratrol derivative or mTor inhibitor (particularly rapamycin) are present in said cell proliferation step.

Depending on the mTOR inhibitor employed, the concentration of the mTOR inhibitor may have to be varied in order to arrive at the desired result. This variation is well within the knowledge of the skilled artisan.

In certain embodiments, the proliferation step is performed until the number of said regulatory T cells has increased at least more than 100-fold.

A third aspect of the present invention relates to a preparation of isolated regulatory T cells specifically reactive towards a CRISPR associated protein polypeptide or towards a homologue thereof, obtained by a method according to the second aspect of the invention.

In certain embodiments, the CRISPR associated protein polypeptide is a Cas9 or Cas12 polypeptide, or a homologue thereof.

In certain embodiments, the CRISPR associated protein polypeptide is a Cas9 polypeptide.

In certain embodiments, the CRISPR associated protein polypeptide is a fusion construct comprising the sequence specificity-providing biological function of the Cas9 polypeptide to another nucleic-acid-modifying enzymatic function.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897, A1IQ68 U2UMQ6 or A0Q7Q2 (Uniprot Entry Ds), and having substantially the same biological activity.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897 or A1IQ68 (Uniprot Entry IDs).

In certain embodiments, >20% of cells in the preparation are specifically reactive towards the CRISPR associated protein polypeptide or towards the homologue thereof, and >80% of such specifically reactive cells are regulatory T cells.

In certain embodiments, >30% of cells in said preparation are specifically reactive towards the CRISPR associated protein polypeptide or towards the homologue thereof, and >80% of such specifically reactive cells are regulatory T cells.

In certain embodiments, >50% of cells in said preparation are specifically reactive towards the CRISPR associated protein polypeptide or towards the homologue thereof, and >80% of such specifically reactive cells are regulatory T cells.

In certain embodiments, >75% of cells in said preparation are specifically reactive towards the CRISPR associated protein polypeptide or towards the homologue thereof, and >80% of such specifically reactive cells are regulatory T cells.

In certain embodiments, the preparation comprises at least 1 million cells.

In certain embodiments, the preparation comprises at least 1 million cells, of which >75% are specifically reactive towards the CRISPR associated protein polypeptide or towards the homologue thereof, particularly >80% of which are Treg cells. The cell preparation is derived from cells originating in a single patient, in other words, the cell preparation is characterized by expressing the same set of HLA molecules; the isolated regulatory T cell of the inventive preparation originate from one patient and not from several patients/blood donors.

To obtain 1 million of cells of which 75% are specifically Cas-reactive and of which >80% are T_{REG} cells, from one patient by mere collecting and isolating cells from a blood sample is hardly possible without risking the life of that patient as approximately 2 Liter blood would be necessary.

A fourth aspect of the invention relates to a preparation of isolated regulatory T cells specifically reactive to a CRISPR associated protein polypeptide, or a homologue thereof, for use in medicine.

In certain embodiments, the CRISPR associated protein polypeptide is a Cas9 or Cas12 polypeptide.

In certain embodiments, the CRISPR associated protein polypeptide is a Cas9 polypeptide.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897, A1IQ68 U2UMQ6 or A0Q7Q2 (Uniprot Entry IDs), and having substantially the same biological activity.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897 or A1IQ68 (Uniprot Entry IDs).

A fifth aspect of the invention relates to a preparation of isolated regulatory T cells specifically reactive to a CRISPR associated protein polypeptide, or a homologue thereof, or towards a homologue of such CRISPR associated protein, wherein said isolated regulatory T cells [each] comprise a transgenic nucleic acid sequence encoding a T cell receptor molecule capable of specifically recognizing an HLA-presented antigen derived from a CRISPR associated protein, or a preparation of isolated regulatory T cells specifically reactive towards a CRISPR associated protein polypeptide, particularly a Cas9 or Cas12 polypeptide, or towards a homologue of such CRISPR associated protein, obtained by a method according to the fourth aspect of the invention for use in a treatment of a condition benefitting from editing a disease related DNA segment.

The disease related DNA segment may be a disease related gene or a disease related non-coding locus.

In certain embodiments, the CRISPR associated protein polypeptide is a Cas9 or Cas12 polypeptide.

In certain embodiments, the CRISPR associated protein polypeptide is a Cas9 polypeptide.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897, A1IQ68 U2UMQ6 or A0Q7Q2 (Uniprot Entry IDs), and having substantially the same biological activity.

In certain embodiments, the CRISPR associated protein polypeptide is a polypeptide having ≥85%, particularly ≥90%, more particularly ≥98%, sequence identity to the amino acid sequence referred to as Q99ZW2, G3ECR1, J7RUA5, Q0P897 or A1IQ68 (Uniprot Entry IDs).

In certain embodiments, the disease of the disease related gene is selected from human papillomavirus-related malignant neoplasm, HIV-1-infection, sickle cell disease, chronic granulomatous disease, multiple myeloma, melanoma, synovial sarcoma, myxoid/round cell liposarcoma, gastrointestinal infection, B cell leukemia, B cell lymphoma, esophageal cancer, neurofibromatosis type 1, tumors of the central nervous system, invasive bladder cancer, hormone refractory prostate cancer, metastatic renal cell carcinoma, metastatic non-small cell lung cancer, gastric carcinoma, nasopharyngeal carcinoma, T cell lymphoma, adult Hodgkin lymphoma, diffuse large B cell lymphoma, β-thalassemia, immunodysregulation polyendocrinopathy enteropathy X-linked (IPEX) syndrome, rheumatic fever, S. pyogenes-associated pharyngitis, S. pyogenes-associated pyoderma, neuroblastoma, acute myelogenous leukemia (AML), acute lymphoblastic leukemia (ALL), chronic myelogenous leukemia (CML), chronic lymphocytic leukemia (CLL), retinoblastoma, Parkinson's disease, Alzheimer's disease, muscular dystrophy, particularly Becker's muscular dystrophy, Duchenne muscular dystrophy, metabolic disease of the liver, familiar osteopetrosis, osteoporosis, osteogenesis imperfecta, Leber's congenital amaurosis, congenital hearing loss, common variable immunodeficiency (CVID), cardiomyopathy and diseases caused by viral infections, particularly by herpes virus infections, more particularly Epstein-Barr virus (EBV) infection, human cytomegalovirus (CMV) infection, herpes simplex infection, human immunodeficiency virus (HIV) infection and human papilloma virus (HPV) infection.

In certain embodiments of the fifths aspect of the invention, the preparation is administered prior to and/or concomitant with administration of a gene therapy agent comprising a CRISPR associated protein, particularly Cas9 or Cas12, or a homologue of such CRISPR associated protein, or of a gene therapy agent comprising a polynucleotide sequence encoding a CRISPR associated protein, particularly Cas9 or Cas12, or a homologue of such CRISPR associated protein.

In certain embodiments, the disease of the disease related gene is selected from immunodysregulation polyendocrinopathy enteropathy X-linked (IPEX) syndrome, rheumatic fever, S. pyogenes-associated pharyngitis and S. pyogenes-associated pyoderma.

In certain embodiments, the preparation is administered prior to and/or concomitant with administration of a gene therapy agent comprising a CRISPR associated protein, or a homologue thereof, or of a gene therapy agent comprising a polynucleotide sequence encoding a CRISPR associated protein, or a homologue thereof.

A sixth aspect of the invention relates to a method for assessing the immunogenicity of a CRISPR-associated protein containing cell, particularly a genome-edited cell. Such method is of use in situations where a risk of an immune reaction is to be assessed in a patient scheduled to undergo treatment with cells that have been manipulated ex-vivo to contain the CRISPR-associated protein. As the probability of an immune response being mounted depends both on the level of immunity pre-existing in the patient, and on the level of expression and presentation of the CRISPR-associated protein by the manipulated cell, the most direct way to predict a (possibly life-threatening or therapy-compromising) immune reaction is to measure the stimulation of the patient's immune cells ex vivo. The method generally follows the principles laid out herein and comprises the steps of
a. providing a cell preparation comprising the patient's T cells,
b. in a stimulation step, contacting said cell preparation with a preparation of cells that were manipulated to comprise and/or express (from a transgene) a CRISPR associated protein, particularly Cas9 or Cas12, or a homologue thereof, whereby activated T cells are generated if the manipulated cells present CRISPR associated protein antigen to a sufficiently primed T cell population;
c. as laid out above, optionally adding an inhibitor of intracellular protein transport, particularly Brefeldin A and/or Monensin, to the cell preparation during the last part of said stimulation step;
d. detecting one or more subpopulations of the activated T cells in the cell preparation exposed to the manipulated cells during the stimulation step by
   contacting said activated T cells with a set of molecular probes specific to regulatory T cells and/or effector T cells, wherein the molecular probes for the two cell preparations are the ones laid out above, and
   contacting said activated T cells with one or more molecular probes specific to activated T cells,
wherein the molecular probes employed to identify the two cell preparations are the ones laid out above,
providing marked activated regulatory T cells and/or marked activated effector T cells;
e. in a quantification step, counting said marked activated regulatory T cells and/or marked activated effector T cells and counting marked regulatory T cells and/or marked effector T cells,
f. assigning a ratio of the number of marked activated regulatory T cells and/or marked activated effector T cells to the number of marked regulatory T cells and/or effector T cells (T_{act}/T_{inact}) to a probability of said cell being immunogenic.

In certain embodiments, the contacting in the stimulation step is performed between 1 h and 25 h, in particular 12 h to 20 h, more particularly 12 h to 18 h.

The invention further encompasses, but is not limited to, the following items:
1. A method for determining a T cell mediated immunity towards a CRISPR associated protein, particularly towards Cas9 or Cas12, or towards a homologue of such CRISPR associated protein, comprising the steps of
   a. providing a cell preparation comprising T cells obtained from a patient;
   b. in a stimulation step, contacting said cell preparation with
      an isolated CRISPR associated protein polypeptide, particularly a Cas9 or Cas12 polypeptide, or a homologue of such CRISPR associated protein, or
      a plurality of peptides, wherein said plurality of peptides represents the amino acid sequence of said CRISPR associated protein polypeptide, particularly said Cas9 or Cas12 polypeptide, or said homologue thereof, or
      a cell comprising a CRISPR associated protein polypeptide, particularly Cas9 or Cas12, or a homologue of such CRISPR associated protein,
   in the presence of antigen presenting cells, particularly in the presence of autologous peripheral blood mononuclear cells,
   providing activated T cells;
   c. optionally, adding an inhibitor of intracellular protein transport, particularly Brefeldin A and/or Monensin, to said cell preparation during the last part of said stimulation step;
   d. in a detection step, detecting one or more subpopulations of said activated T cells by
      contacting said activated T cells with a set of molecular probes specific to activated regulatory T cells, and/or
      contacting said activated T cells with a set of molecular probes specific to activated effector T cells,
   providing marked activated regulatory T cells and/or marked activated effector T cells;
   e. in a quantification step, determining a number of said marked activated regulatory T cells and a number of said marked activated effector T cells, and optionally, a ratio (T_{REG}/T_{EFF}) of the number of said marked activated regulatory T cells to said marked activated effector T cells.
2. A method for determining a nucleic acid sequence encoding a T cell receptor molecule capable of specifically recognizing an HLA-presented antigen derived from a CRISPR associated protein, particularly a Cas9 or Cas12 protein, or from a homologue of such CRISPR associated protein, comprising the steps of
   a. providing a cell preparation comprising T cells obtained from a patient;
   b. in a stimulation step, contacting said cell preparation with
      an isolated CRISPR associated protein polypeptide, particularly a Cas9 or Cas12 polypeptide, or a homologue of such CRISPR associated protein, or
      a plurality of peptides, wherein said plurality of peptides represents the amino acid sequence of said CRISPR associated protein polypeptide, particularly said Cas9 or Cas12 polypeptide, or said homologue thereof, or
      a cell comprising a CRISPR associated protein polypeptide, particularly Cas9 or Cas12, or a homologue of such CRISPR associated protein,
   in the presence of antigen presenting cells, particularly in the presence of autologous peripheral blood mononuclear cells,
   providing activated T cells;
   c. optionally, adding an inhibitor of intracellular protein transport, particularly Brefeldin A and/or Monensin, to said cell preparation during the last part of said stimulation step;
   d. in an isolation step, isolating one or more subpopulations of said activated T cells by
      contacting said activated T cells with a set of molecular probes specific to activated regulatory T cells, and/or
      contacting said activated T cells with a set of molecular probes specific to activated effector T cells,
   and removing a population of CRISPR specific regulatory T cells and/or CRISPR specific activated effector T cells from said preparation;
   e. in a sequence determination step, determining one or more nucleic acid sequences encoding a CRISPR specific T cell receptor molecule comprised in said population of CRISPR specific T cells.
3. The method according to item 1 or 2, wherein in said detection step or said isolation step, said set of molecular probes specific to activated regulatory T cells comprises ligands specific to
   CD3, CD4, CD137, CD154 and CD25, and
   one or more ligands specific to any one of FoxP3, helios and CD127,
   and optionally, one or more ligands specific to any one of CD69, CD71, CD103, CD134, GARP, HLA-DR, IFNγ, IL-10, KLRG1, LAP, SATB1, TGFβ or TNFα.
4. The method according to any one of item the preceding items, wherein in said detection step or said isolation step, a cell is assigned an activated regulatory T cell that is
   positive for CD3, CD4, CD137 and CD25, wherein CD25 is highly expressed, and negative for CD154, and
   positive for FoxP3 and/or positive helios and/or negative for CD127,
   and optionally, positive for any one of CD69, CD71, CD103, CD134, GARP, HLA-DR, IFNγ, IL-10, KLRG1, LAP, SATB1, TGFβ or TNFα.
5. The method according to any one of the preceding items, wherein in said detection step or said isolation step, said set of molecular probes specific to activated effector T cells comprises
   a. ligands specific to CD3, CD4, CD137 and CD154, and optionally, one or more ligands specific to any one of CD69, CD71, CD80, CD86, CD107a, CD134, Granzyme B, HLA-DR, IFNγ, IL-2, KLRG1, Perforin or TNFα; and/or
   b. ligands specific to CD3, CD8 and CD137, and optionally, one or more ligands specific to any one of CD69, CD71, CD80, CD86, CD107a, CD134, Granzyme B, HLA-DR, IFNγ, IL-2, KLRG1, Perforin or TNFα; and/or
   c. ligands specific to CD3, CD4 and CD137 and one or more ligands specific to CD25, FoxP3 and helios, and optionally, one or more ligands specific to any one of CD69, CD71, CD80, CD86, CD107a, CD134, Granzyme B, HLA-DR, IFNγ, IL-2, KLRG1, Perforin or TNFα.
6. The method according to any one of items 1, 3 to 5, wherein in said detection step, a cell is assigned an activated effector T cell that is positive for CD3 and CD137, and
   a. positive for CD4 and CD154 or
   b. positive for CD8 or
   c. positive for CD4 and
      positive for CD25, wherein CD25 is lowly expressed, and/or
      negative for FoxP3 or helios;
   and optionally, positive for any one of CD69, CD71, CD80, CD86, CD107a, CD134, Granzyme B, HLA-DR, IFNγ, IL-2, KLRG1, Perforin or TNFα.
7. The method according to any one of items 1, 5 to 6, wherein said ratio T_{REG}/T_{EFF} is assigned to a probability of said patient reacting to a therapeutic comprising a CRISPR associated protein, or towards a therapeutic comprising a homologue thereof, by a cytotoxic immune response.
8. The method according to item 7, wherein
   a ratio T_{REG}/T_{EFF}<0.5 is assigned to a high risk,
   a ratio 0.5≤T_{REG}/T_{EFF}<1 is assigned to a medium risk,
   a ratio T_{REG}/T_{EFF}≥1 is assigned to a low risk
   of said patient reacting to a CRISPR associated protein, or towards a homologue thereof, by an effector T cell response.
9. The method according to any one of items 2 to 5, wherein the expression of MHC-II isotypes is determined for said patient, and said and said sequences encoding a CRISPR specific T cell receptor molecule are assigned to an MHC-II isotype group.
10. The method according to any one of items 2 to 5 and 9, wherein said method is repeated for a plurality of patients characterized by a shared MHC-II haplotype, and CRISPR specific T cell receptor molecules shared by a significant number of said patients are assigned to an MHC-II matching group.
11. The method according to any one of items 2 to 5 and 9 to 10, wherein only CRISPR specific regulatory T cells are isolated, and nucleic acids encoding CRISPR specific regulatory T cell receptor molecules are determined.
12. The method according to any one of items 2 to 5 and 9 to 11, wherein only CRISPR specific effector T cells are isolated, and nucleic acids encoding CRISPR specific effector T cell receptor molecules are determined.
13. A method for preparing a preparation of regulatory T cells specifically reactive towards a CRISPR associated protein, particularly Cas9 or Cas12, or towards a homologue thereof, comprising the steps of
   a. providing a cell preparation comprising T cells;
   b. in a first isolation step, isolating (non-activated) regulatory T cells using a set of molecular probes specific to non-activated regulatory T cells,
   c. in a stimulation step, contacting said cell preparation with
      an isolated CRISPR associated protein polypeptide, particularly a Cas9 or Cas12 polypeptide, or a homologue of such CRISPR associated protein, or
      a plurality of peptides, wherein said plurality of peptides represents the amino acid sequence of said CRISPR associated protein polypeptide, particularly said Cas9 or Cas12 polypeptide, or said homologue thereof, providing activated T cells in a stimulated cell preparation;
   d. in a second isolation step, isolating activated regulatory T cells from said stimulated cell preparation using
      a set of molecular probes specific to activated regulatory T cells, or
      a set of molecular probes specific to activation-specific marker molecules;
   e. in a cell proliferation step, cultivating said activated regulatory T cells, wherein either one of the first and second isolation step is performed or both first and second isolation steps are performed.
14. A method for preparing a preparation of T cells specifically reactive towards a CRISPR associated protein, particularly Cas9 or Cas12, or towards a homologue thereof, comprising the steps of
   providing a T cell preparation;
   introducing a nucleic acid expression construct into said T cell preparation, yielding a transgene T cell preparation,
   wherein said nucleic acid expression construct encodes a T cell receptor molecule capable of specifically recognizing an HLA-presented antigen derived from a CRISPR associated protein, particularly a Cas9 or Cas12 protein, or from a homologue of such CRISPR associated protein.
15. The method according to item 14, wherein said T cell preparation is a preparation of regulatory T cells.
16. The method according to item 15, wherein said preparation of regulatory T cells is obtained by isolating (non-activated) regulatory T cells from a preparation comprising T cells, using a set of molecular probes specific to non-activated regulatory T cells.
17. The method according to any one of items 13 and 16, wherein in said first isolation step according to item 13 or in the method according to item 16, said set of molecular probes specific to non-activated regulatory T cells comprises ligands specific to CD3, CD4, CD25 and CD127 and/or CD137.
18. The method according to any one of items 13 and 16 to 17, wherein said ligands specific to CD3, CD4 or CD25 are used for positive selection, wherein in case of a ligand specific to CD25 only cells with a high CD25 expression are selected, and wherein said ligands specific to CD127 and/or CD137 are used for negative selection.
19. The method according to any one of items 16 to 18, wherein a transgene T cell preparation is kept under conditions of cell culture in a cell proliferation step.
20. The method according to any one of items 13, 17 and 18, wherein in said second isolation step, said set of molecular probes specific to activated regulatory T cells comprises ligands specific to CD3, CD4, CD137, CD154, CD25 and CD127,
   and optionally, one or more ligands specific to CD69, CD71, CD103, CD134, GARP, HLA-DR, KLRG1 or LAP.
21. The method according to any one of items 13, 17, 18 and 20, wherein in said second isolation step, said set of molecular probes specific to activation-specific marker molecules comprises a ligand specific to CD137 and optionally one or more ligands specific to CD69, CD71, CD103, CD134, GARP, HLA-DR, KLRG1 or LAP.
22. The method according to any one of items 13, 17, 18, 20 and 21, wherein in said second isolation step,
   said ligands specific to CD3, CD4, CD25, CD137, CD69, CD71, CD103, CD134, GARP, HLA-DR, KLRG1 or LAP are used for positive selection, wherein in case of a ligand specific to CD25 only cells with a high CD25 expression are selected;
   said ligands specific to CD127 or CD154 are used for negative selection.
23. The method according to any one of items 13 and 17 to 22, wherein IL-2 and optionally any one of resveratrol, a resveratrol analogue, a resveratrol derivative, or an mTor inhibitor are present in said cell proliferation step.
24. The method according to any one of items 13 and 17 to 22, wherein 50 IU/ml to 5000 IU/ml, particularly 50 IU/ml to 2000 IU/ml, more particularly 200 IU/ml to 1000 IU/ml of IL-2 are present in said cell proliferation step and optionally 50 nM to 150 nM, particularly 100 nM resveratrol, a resveratrol analogue, a resveratrol derivative or mTor inhibitor are present in said cell proliferation step.
25. The method according to any one of items 14 to 19 and 23 to 24, wherein said nucleic acid expression construct encodes a CRISPR specific regulatory T cell receptor molecule
26. The method according to item 14 or 25, wherein said nucleic acid expression construct encodes a CRISPR specific effector T cell receptor molecule
27. A preparation of isolated regulatory T cells specifically reactive towards a CRISPR associated protein polypeptide, particularly a Cas9 or Cas12 polypeptide, or towards a homologue of such CRISPR associated protein, obtained by a method according to any one of items 13, 17, 18 and 20 to 24.
28. A preparation of isolated regulatory T cells specifically reactive towards a CRISPR associated protein polypeptide, particularly a Cas9 or Cas12 polypeptide, or towards a homologue of such CRISPR associated protein, wherein said isolated regulatory T cells [each] comprise a transgenic nucleic acid sequence encoding a T cell receptor molecule capable of specifically recognizing an HLA-presented antigen derived from a CRISPR associated protein.
29. The preparation of isolated regulatory T cells specifically reactive towards a CRISPR associated protein polypeptide according to item 28, wherein
   a. said transgenic nucleic acid sequence was obtained by a method according to any one of items 2 to 5, 9 to 12, and/or
   b. said preparation of isolated regulatory T cells was prepared by a method according to any one of items 14 to 19 and 23 to 26.
30. The preparation according to any one of items 27 to 29, wherein >20%, particularly >30%, more particularly >50%, even more particularly >75% of cells in said preparation are specifically reactive towards said CRISPR associated protein polypeptide or towards said homologue thereof, and >80% of such specifically reactive cells are regulatory T cells.
31. A preparation of isolated regulatory T cells according to any one of items 27 to 30 for use in medicine.
32. A preparation of isolated regulatory T cells according to items 27 to 30 for use in a treatment of a condition benefitting from editing a disease related DNA segment.
33. The preparation for use in a treatment of a condition benefitting from editing a disease related DNA segment according to item 32, wherein the disease is selected from human papillomavirus-related malignant neoplasm, HIV-1-infection, sickle cell disease, chronic granulomatous disease, multiple myeloma, melanoma, synovial sarcoma, myxoid/round cell liposarcoma, gastrointestinal infection, B cell leukemia, B cell lymphoma, esophageal cancer, neurofibromatosis type 1, tumors of the central nervous system, invasive bladder cancer, hormone refractory prostate cancer, metastatic renal cell carcinoma, metastatic non-small cell lung cancer, gastric carcinoma, nasopharyngeal carcinoma, T cell lymphoma, adult Hodgkin lymphoma, diffuse large B cell lymphoma, β-thalassemia, immunodysregulation polyendocrinopathy enteropathy X-linked (IPEX) syndrome, rheumatic fever, S. pyogenes-associated pharyngitis, S. pyogenes-associated pyoderma, neuroblastoma, acute myelogenous leukemia (AML), acute lymphoblastic leukemia (ALL), chronic myelogenous leukemia (CML), chronic lymphocytic leukemia (CLL), retinoblastoma, Parkinson's disease, Alzheimer's disease, muscular dystrophy, particularly Becker's muscular dystrophy, Duchenne muscular dystrophy, metabolic disease of the liver, familiar osteopetrosis, osteoporosis, osteogenesis imperfecta, Leber's congenital amaurosis, congenital hearing loss, common variable immunodeficiency (CVID), cardiomyopathy and diseases caused by viral infections, particularly by herpes virus infections, more particularly Epstein-Barr virus (EBV) infection, human cytomegalovirus (CMV) infection, herpes simplex virus infection, human immunodeficiency virus (HIV) infection and human papilloma virus (HPV) infection.
34. The preparation for use in a treatment of a condition benefitting from editing a disease related DNA segment according to item 27, 28 or 33, wherein said preparation is administered prior to and/or concomitant with administration of a gene therapy agent comprising a CRISPR associated protein, particularly Cas9 or Cas12, or a homologue of such CRISPR associated protein, or of a gene therapy agent comprising a polynucleotide sequence encoding a CRISPR associated protein, particularly Cas9 or Cas12, or a homologue of such CRISPR associated protein.
35. A preparation of isolated regulatory T cells for use in medicine according to item 31, or for use in a treatment of a condition benefitting from editing a disease related DNA segment according to items 32 to 34, wherein the preparation for administration to a patient characterized by a ratio T_{REG}/T_{EFF} of regulatory T cells specific for a CRISPR associated protein to effector T cells specific for a CRISPR associated protein to effector T cells,
   particularly wherein said patient is characterized by a ratio T_{REG}/T_{EFF}<1,
   more particularly wherein said patient is characterized by a ratio T_{REG}/T_{EFF}<0,5.
36. A preparation of isolated regulatory T cells for use in medicine according to item 31, or for use in a treatment of a condition benefitting from editing a disease related DNA segment according to items 32 to 34, wherein the preparation of T cells is autologous (provided for use in the same patient having been the source of the T cell preparation into which the transgenic nucleic acid expression construct encoding a T cell receptor molecule capable of specifically recognizing an HLA-presented antigen derived from a CRISPR associated protein had been transferred).
37. A preparation of isolated regulatory T cells for use in medicine according to item 31, or for use in a treatment of a condition benefitting from editing a disease related DNA segment according to items 32 to 34, wherein
   the preparation is provided for use in a patient having a known MHC-II patient isotype, and
   the nucleic acid expression construct encoding said T cell receptor molecule capable of specifically recognizing an HLA-presented CRISPR antigen is selected from an MHC-II matching group assigned to said MHC-II patient isotype.

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 shows a ubiquitous peripheral SpCas9-specific T cell response within human donors. SpCas9-specific human CD3⁺ T cells can be identified after short-term ex vivo stimulation. PBMCs were stimulated with SpCas9 whole protein for 16 h. Frequencies of T cell response were assessed by flow cytometry. (a) Representative FACS plots show SpCas9-induced activation defined by CD137 expression of CD8⁺ and CD8⁻ T cells in comparison to unstimulated control. (b) Gating of single alive CD3⁺ T cells and dissection into CD4⁺ and CD8⁺ T cells. Representative FACS plots of SpCas9-induced CD137 and CD154 expression as well as IFN-γ, TNF-α and IL-2 production are shown. (c) Representative FACS plots of IFN-γ and TNF-α production within SpCas9-activated CD4⁺CD137⁺ and CD8⁺CD137⁺ T cells. (d) SpCas9, CMV-pp65 or SEB-induced activation (CD4⁺ CD8⁺), defined by CD137 expression, compared to unstimulated controls (unstimulated n=48 healthy donors; SpCas9 n=48 healthy donors; CMV-pp65 n=35 healthy donors; SEB n=26 healthy donors). (e) Background subtracted CD137 expression to SpCas9 whole protein by CD4+ and CD8+ T cells. (f) SpCas9-induced expression of CD154, TNF-α, IFN-γ and IL-2 within activated CD4⁺CD137⁺ and CD8⁺CD137⁺ T cells. (n=24; horizontal lines within graphs indicate medians.)

FIG. 2 shows that a SpCas9-specific T cell response contains a substantial proportion of regulatory T cells. Identification of T_{EFF} and T_{REG} phenotypes within CD137⁺ T cells after 16 h stimulation of human PBMCs with SpCas9 whole protein. (a) Representative FACS plots show FoxP3 expression of T_{REG}-defining markers CD25, FoxP3, CTLA-4 and CD127 within SpCas9-activated CD4⁺CD137⁺ and CD4⁻CD137⁺ T cells. The overlay highlighted in black represents CD25⁺FoxP3⁺ of CD137⁺ T cells. (b) Contribution to SpCas9-induced CD4⁺CD137⁺ T cell response by T_{EFF} and CD25⁺Foxp3⁺ T_{REG} phenotypes. (c) Overlay contour plots of a representative donor demonstrate Tbet⁺ (grey) and FoxP3⁺ (black) T cells within SpCas9-induced T cell activation defined by CD137 and CD154 expression. (d) Gating of CD4⁺ T_{REG} within SpCas9-induced CD4⁺CD137⁺ T cells and (e) corresponding CD154 expression and cytokine production within CD4⁺CD137⁺ T_{REG} (black and T_{EFF} (grey. (f) Summary of accumulated cytokine production within bulk CD4⁺CD137⁺ T cells, CD4⁺CD137⁺ T_{EFF} (CD25⁻FoxP3⁻) and CD4⁺CD137⁺ T_{REG} (CD25⁺FoxP3⁺). (n=24; horizontal lines within graphs indicate median values.)

FIG. 3 shows a balanced effector/regulatory T cell response to SpCas9 whole protein. (a) Relation of antigen-reactive T_{REG} to CD4⁺ T_{EFF} shown for SpCas9 whole protein, CMVₚₚ₆₅ peptides and SEB stimulation. Antigen-reactive T_{REG} and T_{EFF} were defined according to gating strategy presented in FIG. 2d. Ratio was calculated by dividing the frequency of T_{REG} by the proportion of T_{EFF} within CD4⁺CD137⁺ antigen-reactive cells. (b) Relation of antigen-reactive T_{REG} to CD8⁺T_{EFF} shown for SpCas9 whole protein, CMVₚₚ₆₅ peptides and SEB stimulation. Ratio was calculated by dividing the frequency of T_{REG} by the proportion of T_{EFF} within CD8⁺CD137⁺ antigen-reactive cells.

FIG. 4 shows that ex vivo stimulation with SpCas9 whole protein induces polyfunctional effector CD4⁺ and CD8⁺ T cell responses. (a) Experimental design for ex vivo detection of SpCas9-specific T cell responses. (b) Representative gating strategy for defining alive CD3⁺CD4⁺ and CD3⁺CD8⁺ T lymphocytes. Lymphocytes were gated based on the FSC versus SSC profile and subsequently gated on FSC (height) versus FSC to exclude doublets. (c and d; summarized in e and f) Representative FACS images show SpCas9-induced activation defined by CD137 expression plotted against CD154, IFN-γ, TNF-α and IL-2 for CD4⁺ and CD8⁺ T cells in comparison to CMVₚₚ₆₅-stimulated and SEB-stimulated PBMCs. (SpCas9: n=24, CMVₚₚ₆₅: n=12, SEB: n=6. Horizontal lines within graphs indicate medians.)

FIG. 5 shows that SpCas9- and viral CMVₚₚ₆₅-reactive CD4⁺ and CD8⁺ T cells phenotypically show a memory profile. (a) Strategy for defining T cell subsets from PBMCs according to the expression of CD3⁺ CD45RO⁺ and CCR7⁺ within CD4⁺ and CD8⁺ T cells. Dissection of the T cell differentiation profile into the following subsets: Naïve T cells (T_{NAIVE}: CCR7⁺CD45RO⁻), central memory (T_{CM}: CCR7⁺CD45RO⁺), effector memory (T_{EM}: CCR7⁻CD45RO⁺) and terminally differentiated effector T cells (T_{EMRA}: CCR7⁻CD45RO⁻). (b) Strategy for defining T cell differentiation phenotypes applied to antigen-reactive CD4⁺CD137⁺ and CD8⁺CD137⁺ T cells after SpCas9 or human CMVₚₚ₆₅ PBMCs stimulation. Summarized phenotypical distribution of (c) bulk un-stimulated, (d) SpCas9-reactive (CD137⁺) and (e) CMVₚₚ₆₅-reactive (CD137⁺) CD4⁺ and CD8⁺ T cells. Flow cytometric analysis of PBMCs from a representative donor. (SpCas9: n=24. CMVₚₚ₆₅: n=10. Horizontal line in graphs indicates median value.)

FIG. 6 shows that SpCas9-reactive CD4⁺CD137⁺ regulatory T cells show a memory phenotypic profile. (a) Gating strategy for the identification of T_{REG} phenotypes within the CD4⁺ T cell response. (b) Summary of T_{REG}-defining markers CD25, FoxP3, CTLA-4 and CD127 within SpCas9-activated CD4⁺CD137⁺ and CD8⁺CD137⁺ T cells. (c and d) Summary of T cell differentiation phenotypes within SpCas9-reactive CD4⁺CD137⁺FoxP3⁻ T_{EFF} and CD25⁺Foxp3⁺ T_{REG}. (n=24. Horizontal lines in graphs indicate median values.)

FIG. 7 shows that SpCas9-induced CD137 and CD154 expression correlate with lineage determining transcription factor pattern. The SpCas9-induced activation pattern on CD4⁺ was dissected according to CD137 and CD154 expression levels: (1): CD137⁻, (2) CD137⁺CD154⁺, (3) CD137^{high} CD154⁻ and (4) CD137^{dim}CD154⁻. SpCas9-reactive CD8⁺ T cells were defined through CD137 expression. Identification of Tbet (T_{EFF}) and FoxP3 (T_{REG}) transcription factors within (a) the CD4⁺ T cell response (1 to 4) and (b) the CD8⁺ T cell response to 16 h stimulation of human PBMCs with SpCas9 whole protein. (c and d) Summary of Tbet and FoxP3 expression within SpCas9-activated CD3⁺ T cells with designated activation pattern (CD4⁺: 2 to 4; CD8⁺: CD137⁺). (n=6; horizontal lines within graphs indicate median values.)

FIG. 8 shows that SpCas9-reactive CD4⁺ regulatory T cells are CD137^{dim} and lack CD154 expression and effector cytokine production. SpCas9-induced activation pattern on CD4⁺ T cells was dissected according to CD137 and CD154 expression levels: (1): CD137⁻, (2) CD137⁺CD154⁺, (3) CD137^{high} CD154⁻ and (4) CD137^{dim}CD154. (a) Representative FACS plots for SpCas9-induced activation pattern (1-4) and corresponding (b) T_{REG} phenotype (CD25⁺Foxp3⁺) and (c and d) effector cytokine production. Overlay demonstrates T_{REG} contribution to the SpCas9-induced T cell response (red). (e) Summary of accumulated cytokine production within T cells with designated activation pattern (1 to 4).

FIG. 9 shows a flow cytometric enrichment of SpCas9-reactive T_{EFF} and T_{REG}. PBMCs were cultured for 16 h in the presence of 8 μg/ml SpCas9 whole protein and 1 μg/ml CD40-specific antibody. (a) SpCas9-specific T_{REG}/T_{EFF} and un-stimulated pc T_{REG}/T_{EFF} were enriched by FACSorting according to the incremental gating of CD3^{+→}CD4⁺ or CD8^{+→}CD137^{+/-}CD154^{+/-} or CD137^{+/-→}CD25^{high/low}CD127^{+/-}. Post-sorting purity is shown in lower panels for CD4⁺CD137⁻CD154⁻CD25^{high} CD127⁻ (pc T_{REG}), CD4⁺CD137⁻CD154⁻CD25^{low} and CD8⁺CD137⁻CD154⁻CD25^{low} (pc T_{EFF}), CD4⁺CD137⁺CD154⁻CD25^{high} CD127⁻ (SpCas9 T_{REG}) and CD4⁺CD137⁺CD154⁺CD25^{low}, CD4⁺CD137⁺CD154⁻CD25^{low} and CD8⁺CD137⁺ (SpCas9 T_{EFF}). Representative flow cytometric images shown. (n=2). (b) Experimental design for expansion and re-stimulation of enriched SpCas9-reactive T_{EFF} and SpCas9-reactive T_{REG} and respective pc control populations.

FIG. 10 shows an expansion of SpCas9-reactive T cells. Antigen-specific readout for SpCas9-reactive ex vivo isolated and expanded T cells. Cultured SpCas9-specific T_{EFF} and T_{REG} were analysed at day 10 for expression of effector molecules in response to stimulation with SpCas9 whole protein loaded autologous moDCs for 6 h at a ratio of 10:1. Following stimulation, we analysed the expression of CD3, CD4, CD8, CD25, intracellular IFN-γ, TNF-α, IL-2 and FoxP3. (a) CD4 to CD8 ratio, (b) CD25 and FoxP3 expression, (c) TNF-α and IFN-γ and (d) IFN-γ and IL-2 production within designated populations upon different stimuli (SpCas9, CMVₚₚ₆₅ and control).

FIG. 11 shows DNA methylation analysis of the T_{REG}-specific demethylation region (TSDR) in SpCas9-reactive ex vivo isolated CD137⁺CD154^{+/-}CD25⁻CD127⁺FoxP3⁻ (T_{EFF}) and CD137⁺CD154⁻CD25⁺CD127⁻FoxP3⁺ T cells (T_{REG}) (average TSDR demethylation: T_{REG} 86.3%; T_{EFF} 1.39%; n=5 HD). SpCas9-reactive T_{EFF} and T_{REG} isolation: PBMCs were cultured in the presence of 5 μg/ml SpCas9 whole protein and 1 μg/ml CD40-specific antibody for 16 h. SpCas9-reactive T_{REG}/T_{EFF} were enriched by FACSorting according to FIG. 9. Shown are mean values. Statistical analysis by two-tailed paired T-test. *=p<0.05

FIG. 12 shows that SpCas9-reactive regulatory T cells suppress their SpCas9-reactive effector counterpart. (a) Representative FACS plots of IFN-γ production versus FoxP3⁺ expression within SpCas9-activated CD4⁺CD137⁺ and CD4⁺CD137⁺CD154⁺ T cells in the presence or absence of 15 μg/mL αHLA-DR blocking antibody (αMHC-II) or following CD25⁺ depletion after 16 h stimulation of human PBMCs with SpCas9 whole protein, CMVₚₚ₆₅ or SEB. (n=6 HD) (b) Antigen-reactive CD4⁺ T cell proportion and function in the presence of αMHC-II. The dotted line indicates normalized antigen-induced CD4⁺ T cell response w/o αMHC-II shown as %-Δ CD4⁺CD137⁺, CD4⁺CD137⁺CD154⁺, CD4⁺CD137⁺CD154⁺accumulated-cytokine⁺ and CD4⁺ CD137⁺CD25⁺FOXP3⁺ T_{REG}. (C) Antigen-reactive CD4⁺ T cell proportion and function following CD25 depletion from PBMCs. The dotted line indicates normalized antigen-induced CD4⁺ T cell response w/o CD25 depletion shown as %-Δ CD4⁺CD137⁺, CD4⁺CD137⁺CD154⁺ and CD4⁺CD137⁺CD154⁺accumulated-cytokine⁺. The %-Δ CD4⁺CD25⁺FoxP3⁺ T_{REG} indicates successful depletion within the treated condition. The experiment was performed in n=12 HD as biologically independent samples/independent experiments. Mean±s.e.m. is shown; D'Agostino & Pearson normality test was performed. Wilcoxon matched pairs test or paired two-tailed T test were performed depending whether data was distributed normally or not. *=p≤0.05; (d; summarized in e) SpCas9-reactive CD4⁺ T_{REG} suppress their SpCas9-reactive T_{EFF} counterpart dose-dependently. SpCas9-reactive T_{REG} and T_{EFF} and polyclonal (pc) T_{EFF} were enriched according to FIG. 9. T_{EFF} proliferation (blue) for SpCas9-reactive (left) and pc (right) T_{EFF} cells is shown by CFSE-dilution following 96 h culture in the presence or absence of SpCas9-reactive T_{REG} (red) at ratios 1:1 and 1:5. SpCas9-reactive T cells were solely activated once prior to FACSorting. Pc T_{EFF} were stimulated with anti-CD3/CD28-coated microbeads (n=6 HD). (f) Culture supernatants were harvested for cytokine measurements following 96 h culture (MSD multiplex analysis). Cytokine concentrations in supernatants of SpCas9-reactive T_{EFF} and T_{REG} cultures are shown: IFN-γ, TNF-α, IL-2 and IL-10 (n=6 HD). Mean±s.e.m. are shown. Kolmogorov-Smirnov test was performed to evaluate Gaussian distribution. Depending on normality testing either two-tailed paired T test or Wilcoxon matched-pairs test was employed. *=p s 0.05 (g) Suppression of cytokine production in co-cultures of SpCas9-reactive T_{REG} and T_{EFF} is shown. Experiment was performed in n=6 individual HD. Mean±s.e.m are shown. (h; summarized in i) Assessment of SpCas9-reactive T cell mediated cytotoxicity by flow cytometric VITAL assay. Specific cytotoxic killing of SpCas9-transfected targets by SpCas9-reactive T_{EFF}. Transfected LCLs expressing SpCas9 and GFP (LCLs-SpCas9⁺GFP⁺) served as a SpCas9-positive target, while unmodified DDAO⁺LCLs were used as control non-targets. GFP served as a reporter for SpCas9 expression. Cells were co-cultured at T cell/target cell ratios of 10:1, 1:1 and 1:10 for 16 h. Samples without T cells, containing only targets and non-targets (LCLs-SpCas9⁺GFP⁺/LCLs) served as internal control. The mean percent survival of LCLs-SpCas9⁺GFP⁺ target cells was calculated relative to LCL controls. The experiment was performed in n=6 individual healthy donors as biologically independent samples/independent experiments. Mean±s.e.m are shown.

FIG. 13 shows the assessment of SpCas9-reactive T cell-mediated cytotoxicity by flow cytometric VITAL assay. Specific cytotoxic killing of SpCas9-transfected targets by SpCas9-reactive Teff cells s. Transfected LCLs expressing SpCas9 and GFP (LCLs-SpCas9+GFP+) served as a SpCas9-positive target, while unmodified N,N-dimethyldodecylamine N-oxide (DDAO)+ LCLs were used as control nontargets. GFP served as a reporter for SpCas9 expression. Cells were cocultured at T cell/target cell ratios of 10:1, 1:1, and 1:10 for 16 h. Samples without T cells, containing only targets and nontargets (LCLs-SpCas9+GFP+/LCLs), served as the internal control. On the right: Quantification of the SpCas9-reactive T cell-mediated cytotoxicity for n=6 donors according to the experimental setup.

FIG. 14 shows that other CRISPR-associated protein-reactive T cell responses contain a substantial proportion of T_{REG}. a, Representative FACS images show identification T_{REG} phenotype within CD137⁺ T cells after 16 h stimulation of human PBMCs with whole proteins for SpCas9, SaCas9, Cpf1, and CMVₚₚ₆₅ and SEB. b, Quantified data according to c. (n=5 individual HD) Mean is shown. The experiments were performed with dedicated amount of individual healthy donors (n) as biologically independent samples/independent experiments.

FIG. 15 shows ex vivo stimulations with CRISPR-associated whole proteins SpCas9 and SaCas9, as well as Cpf1 induce polyfunctional effector CD4⁺ and CD8⁺ T cell responses. a, Representative FACS images show SpCas9-induced activation defined by CD137 expression plotted against CD154 for CD4⁺ and CD8⁺ T cells in comparison to unstimulated, SaCas9-stimulated, Cpf1-stimulated and SEB-stimulated PBMCs. Short-term ex vivo stimulation with SpCas9 whole protein, SaCas9 whole protein, Cpf1 whole protein and SEB. PBMCs were stimulated for 16 h. Frequencies of T cell response were assessed by flow cytometry. b, Quantified data according to a. (n=5 HD) Mean is shown. c, Background subtracted CD137 expression to SpCas9, SaCas9, Cpf1 and SEB by CD4⁺ and CD8⁺ T cells. (n=5 HD) Mean is shown. d, Summary of background subtracted CD137⁺cytokine⁺ (CD137⁺→accumulated CD154⁺TNF-α⁺IFN-γ⁺IL-2⁺) expression to SpCas9, SaCas9 and Cpf1 whole proteins by CD4⁺ and CD8⁺ T cells. (n=5 HD) Mean is shown. The experiments were performed with dedicated amount of individual healthy donors (n) as biologically independent samples/independent experiments.

FIG. 16 shows SpCas9-reactive T_{REG} frequencies do not diminish when stimulated in absence of T_{EFF}. a, Experimental design for the detection of SpCas9-reactive T_{REG} responses within PBMC and paired bulk pre-enriched T_{REG} to exclude unspecific activation of bystander T_{REG} through SpCas9-reactive T_{EFF}. Stimulation of FACS-enriched bulk T_{REG} with SpCas9-loaded monocytes and B cells in the absence of T_{EFF}. T_{REG} were enriched in bulk by FACS according to the cell surface expression of CD4⁺CD25⁺CD127⁻ (FIG. 9). Sorted T_{REG} were rested over-night at 37° C. in humidified incubators and 5% CO₂ and subsequently stimulated with 5 μg/ml SpCas9-pulsed monocytes (sorted according to SSC/FSC profile) and B cells (sorted CD3⁻ fraction) for 16 h. PBMC were stimulated in parallel with 5 μg/ml SpCas9 whole protein for 16 h. b, Representative FACS images show SpCas9-induced CD4⁺ T cell activation within bulk sorted T_{REG} defined by CD137 expression plotted against FoxP3 in comparison to frequencies within a paired PBMC sample. (n=6 individual HD) c,d, Quantified data according to e and f. Frequencies of CD137⁺CD25⁺FOXP3⁺ T_{REG} within CD4⁺ T cells (g) and CD137⁺ within CD4⁺CD25⁺FoxP3⁺ T_{REG} (h) following SpCas9 whole protein stimulation. Connecting lines indicate paired samples from the same donors. (n=6 HD). We did not observe a significant decrease in SpCas9-reactive T_{REG} frequencies when sorted T_{REG} are stimulated in the absence of T_{EFF} cells. Thus, SpCas9-reactive T_{REG} cells are not activated through bystander SpCas9-reactive CD4⁺/CD8⁺ T_{EFF} cells. The experiments were performed with dedicated amount of individual healthy donors (n) as biologically independent samples/independent experiments.

FIG. 17 shows that short-term ex vivo stimulation with SpCas9 whole protein or SpCas9 overlapping peptide scans (15 amino acids length+11 amino acids overlap; 340 peptides) induces effector CD4⁺ and CD8⁺ T-cell responses. PBMCs were stimulated with SpCas9 whole protein or SpCas9 overlapping peptide scans each at indicated concentrations for 16 h. Frequencies of T-cell response were assessed by flow cytometry. Lymphocytes were gated based on the FSC versus SSC profile and subsequently gated on FSC (height) versus FSC to exclude doublets. a SpCas9-induced expression of CD137 and CD154 within activated CD4⁺ T cells (n=24 HD). b SpCas9-induced expression of CD137 within activated CD8⁺ T cells (n=24 HD). The experiments were performed with dedicated amount of healthy individuals (n) as biologically independent samples/independent experiments.

### EXAMPLES

### Example 1: Detecting CRISPR Associated Nuclease-Directed T Cell Response

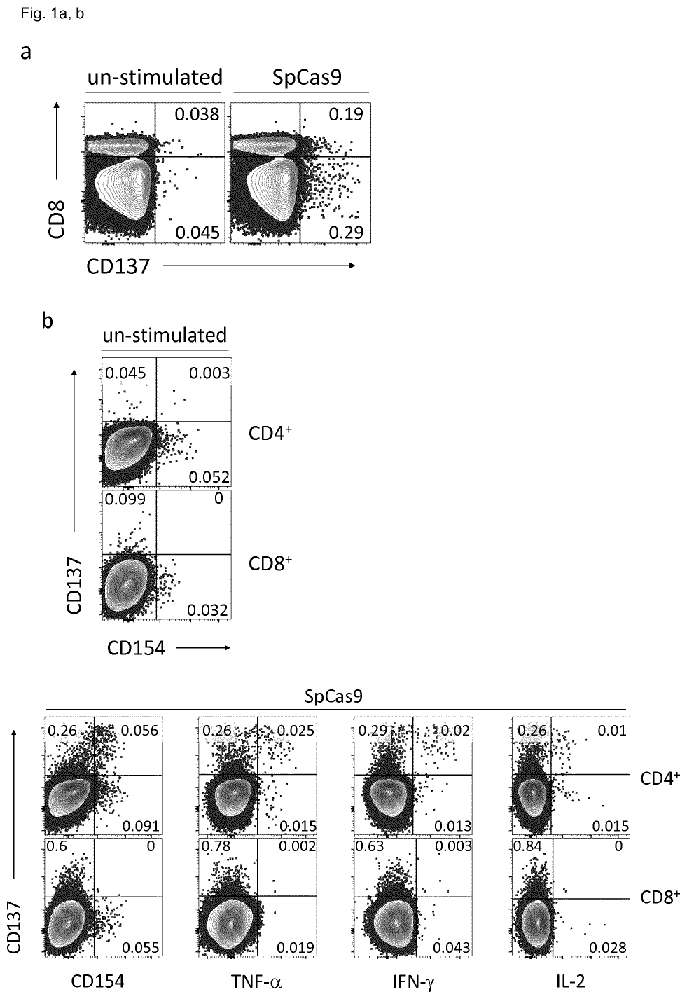

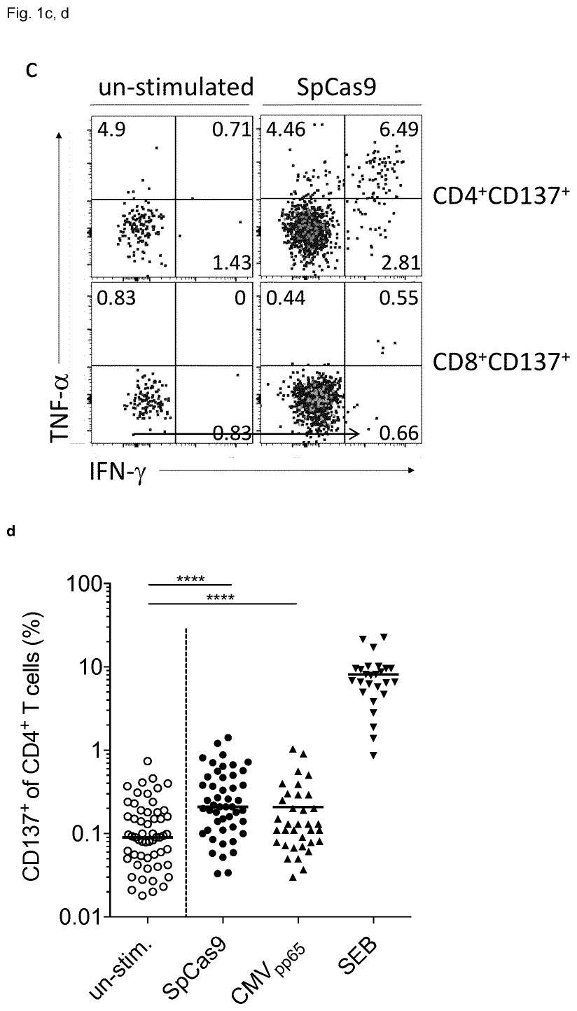

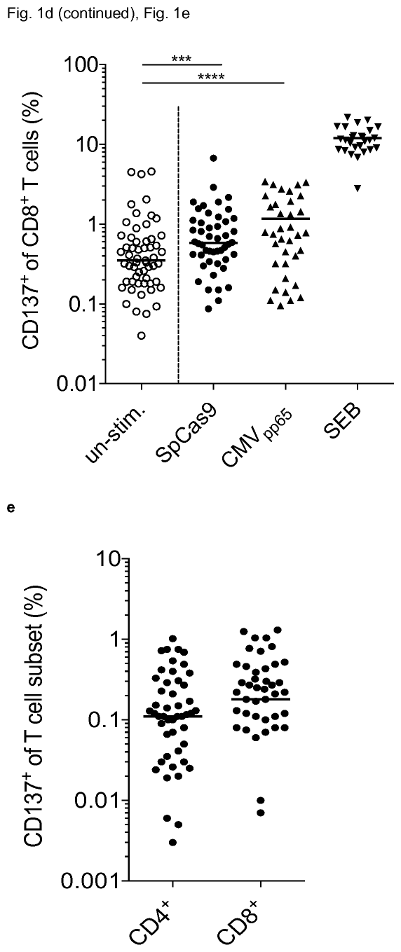

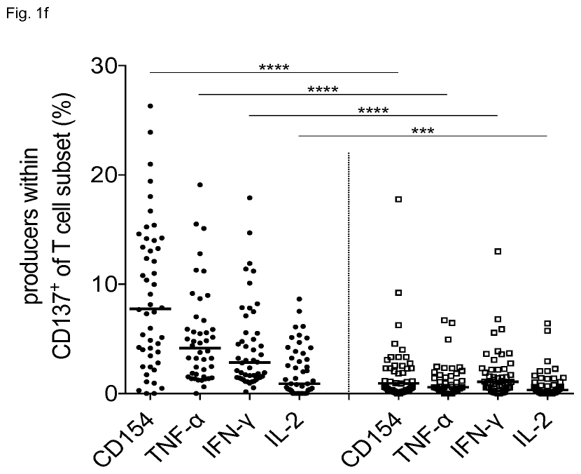

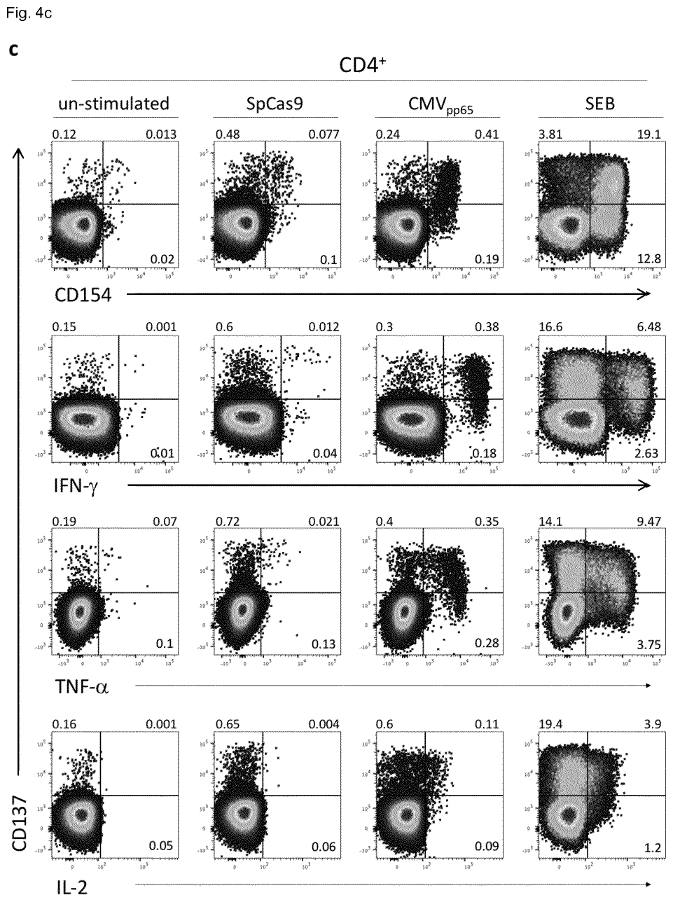

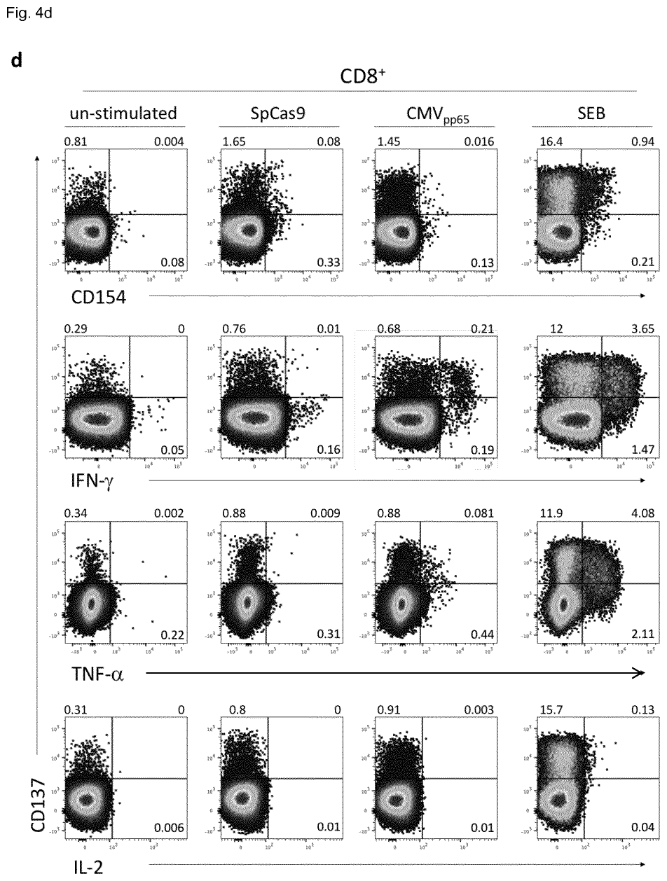

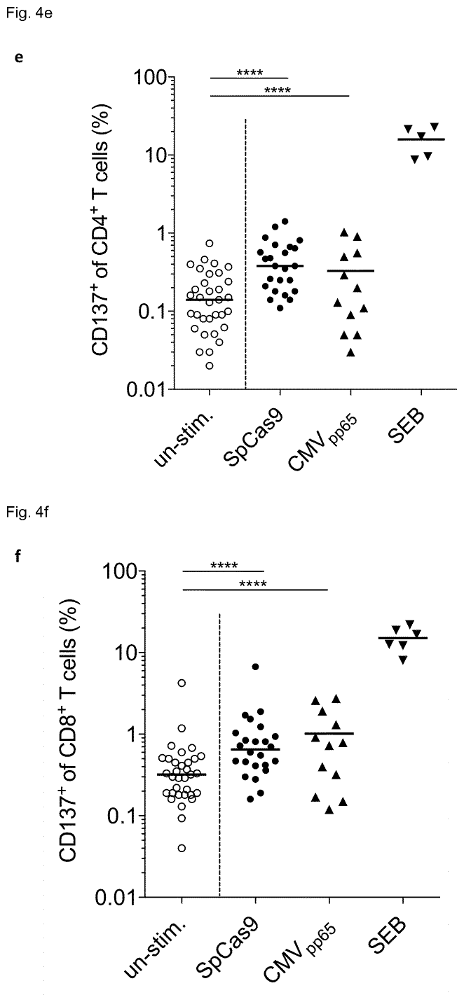

For detection of a putative SpCas9-directed T cell response, human peripheral blood mononuclear cells (PBMCs) were stimulated with recombinant SpCas9 and the reactivity of CD3⁺4⁺/8⁺ T cells was analysed by flow cytometry with a set of markers for T cell activation (CD137, CD154) and effector cytokine production (IFN-γ, TNF-α, IL-2) (FIG. 1a, b, FIG. 4) (Frentsch, M. et al. 2005, Nat. Med. 11, 1118-1124; Wolfl, M. et al. 2007, Blood 110, 201-10). The inventors relied on protein uptake, processing and presentation of SpCas9 peptides by professional antigen-presenting cells (APCs) to both MHC I- and II within the PBMCs. Intriguingly, 96% of all healthy human donors evaluated showed specific memory/effector T cell activation upon SpCas9 stimulation indicated by CD137 (4-1BB) upregulation in both, CD4 and CD8, T cell compartments (FIG. 1a, b, d, e, FIG. 4). After subtraction of background an average of 0.24% (range 0.03-1.3%) expressed CD137 within CD4⁺ and CD8⁺ T cells (FIG. 1e). By multiparameter analysis at single cell level, Cas9-specific multi-potent T_{EFF} expressing at least one or even more effector cytokines (CD4⁺>CD8⁺ T cells) (FIG. 1b, c, f) were detected.

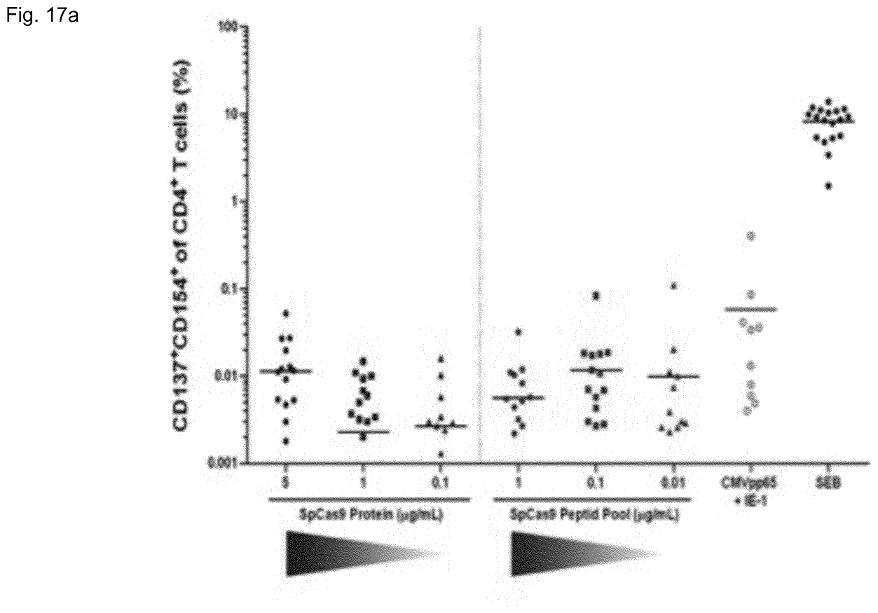

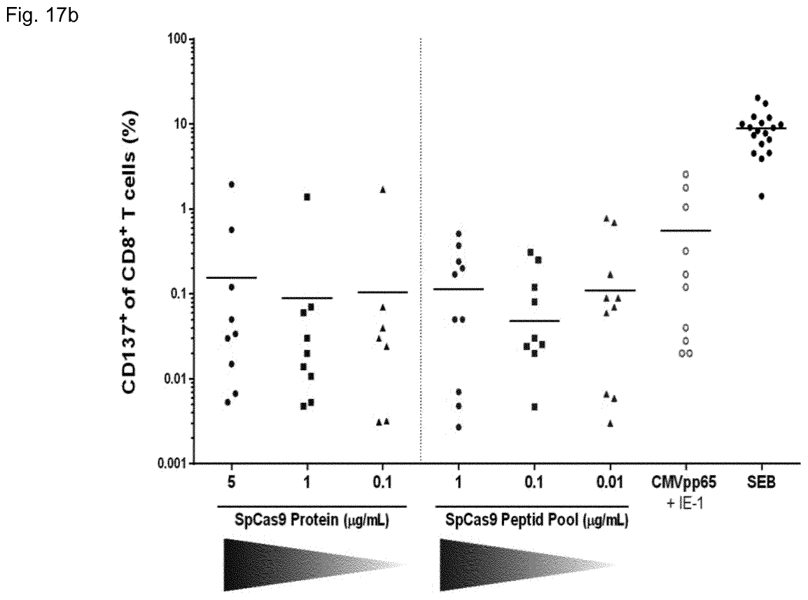

Cas-directed T cell responses can be provoked by stimulation with oligopeptide pools. The inventors performed side-by-side stimulations with SpCas9 whole protein and SpCas9 overlapping peptide libraries in SpCas9-sensitized donors and detected similar frequencies as shown in FIG. 17.

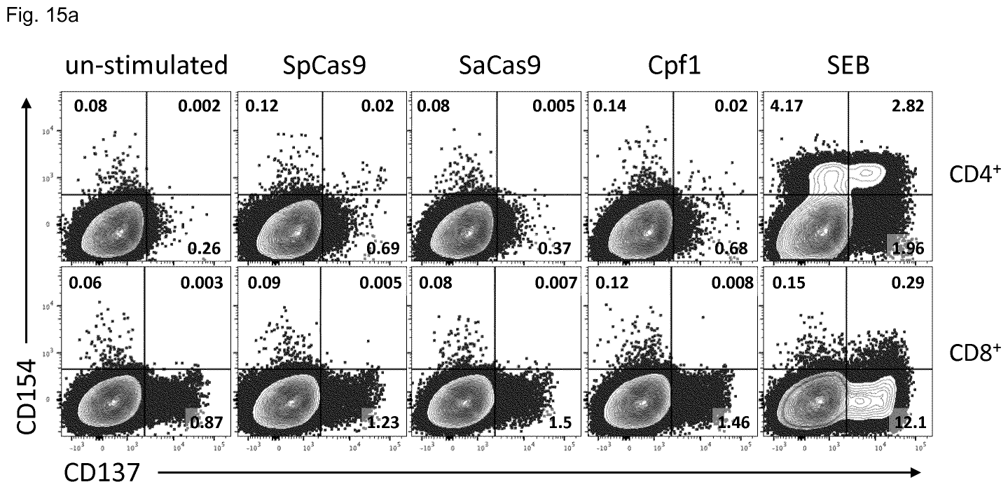

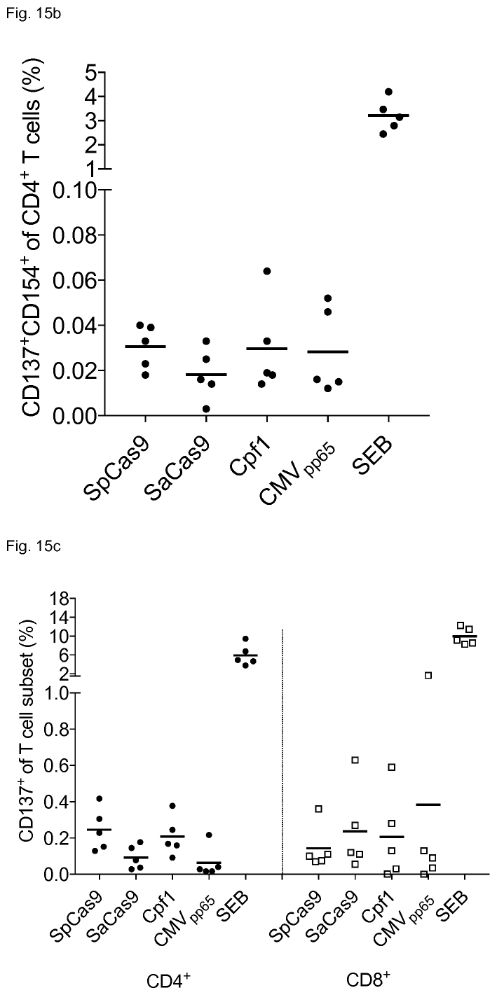

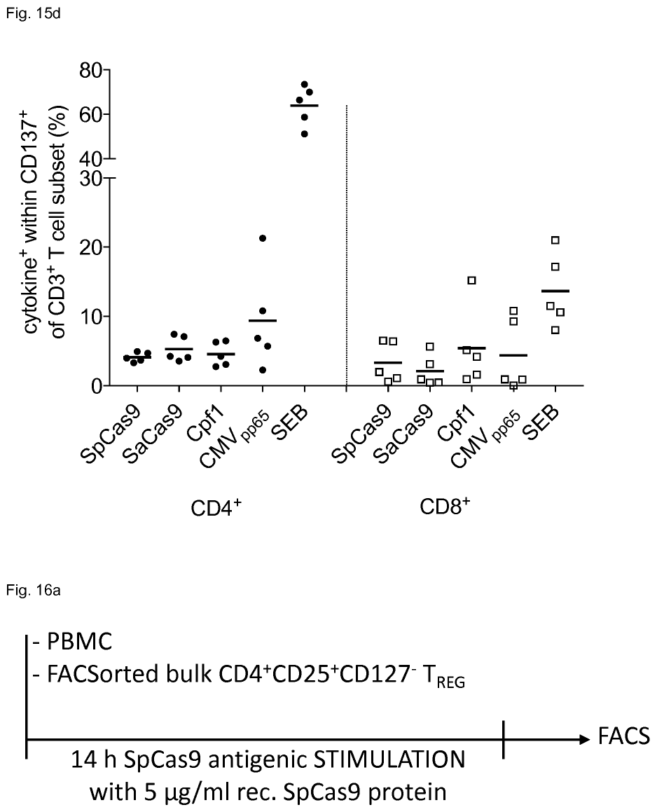

When PBMCs were stimulated with recombinant proteins from other CRISPR-associated nucleases like SaCas9 (derived from Staphylococcus aureus) or AsCpf1 (derived from Acidaminococcus species), a pre-existing T cell response similar to the one toward SpCas9 was detected. (FIG. 15)

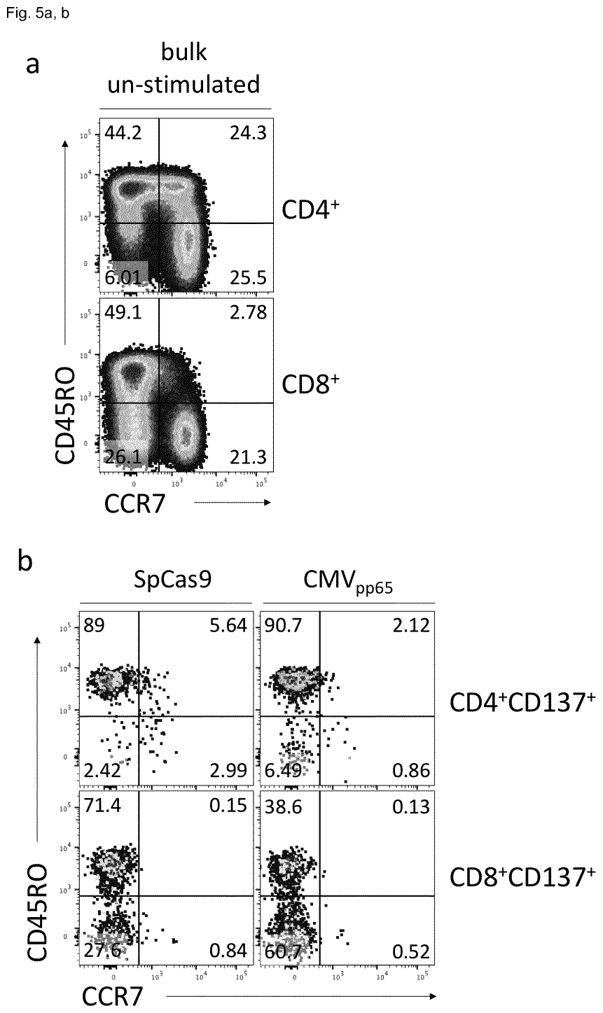

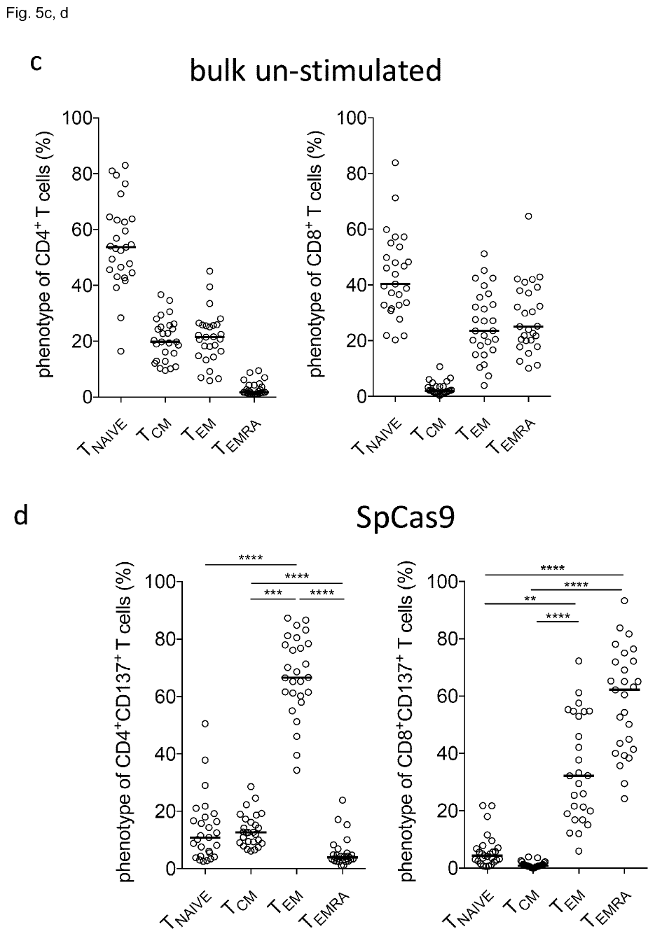

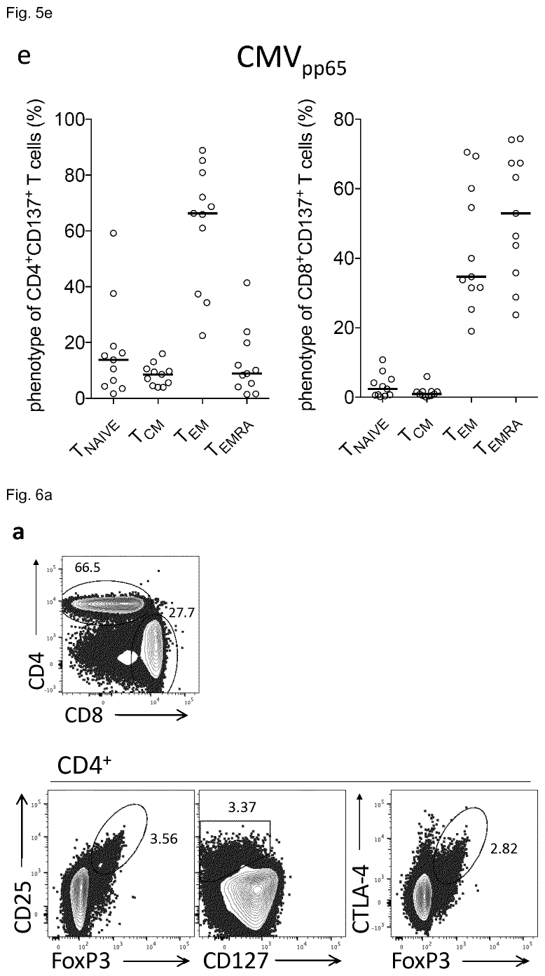

The expression of the lymph node homing receptor CCR7 and the leucocyte common antigen isoform CD45RO allows for dissection of the reactive T cell subsets (FIG. 5a) (Sallusto, F. et al. 1999, Nature 401, 708-712). Accordingly, the inventors discovered that the majority of SpCas9-reactive T cells belongs to the effector-memory (CD4⁺ and CD8⁺) and terminally differentiated effector memory effector cells (T_{EMRA}) (CD8⁺) pool implying repetitive previous exposure to SpCas9, comparable with memory T cell response to the frequently reactivated cytomegalovirus (CMV) (FIG. 5b-e) (Schmueck-Henneresse, M. et al. 2015, J. Immunol. doi:10.4049/jimmunol.1402090). The few cells within the naïve compartment might be related to stem cell memory T cell subset within this population.

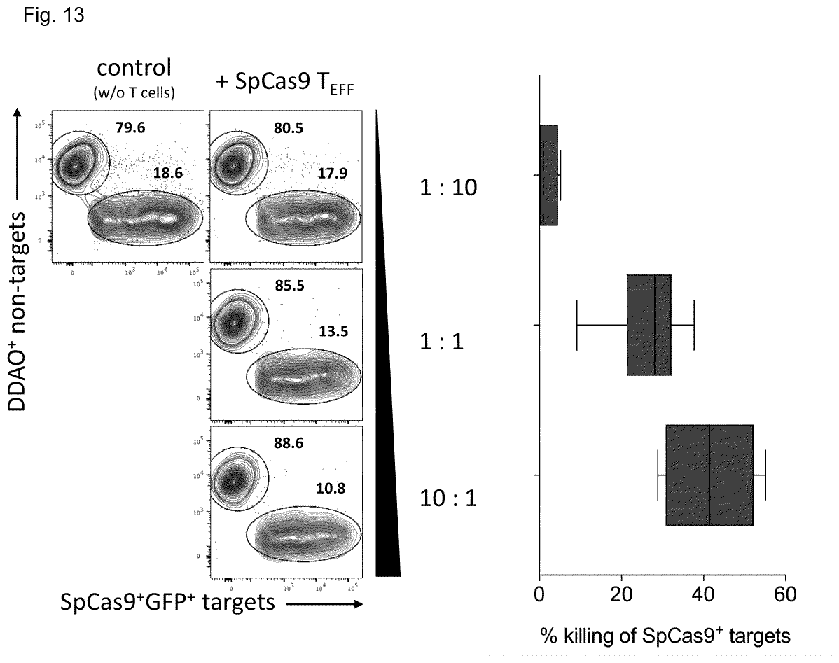

The results imply an almost ubiquitous pre-primed T_{EFF} response towards SpCas9, which could have immediate detrimental effects on tissues edited with a SpCas9-related system as those cells can immediately migrate to the targeted tissue. (As simulated in FIG. 13, enriched SpCas9-specific T cells can lyse SpCas9-expressing cells in a dose dependent manner.) Similarly, common alternatives for SpCas9 like Cas9 from Staphylococcus aureus (SaCas9) or Cas12a from Acidaminoccus species (AsCpf1) are derived from commensal bacteria. They are also at risk for pre-primed memory T cell responses according to the results shown here (FIG. 13).

### Example 2: Dissecting the CRISPR Associated Nuclease-Directed T Cell Response

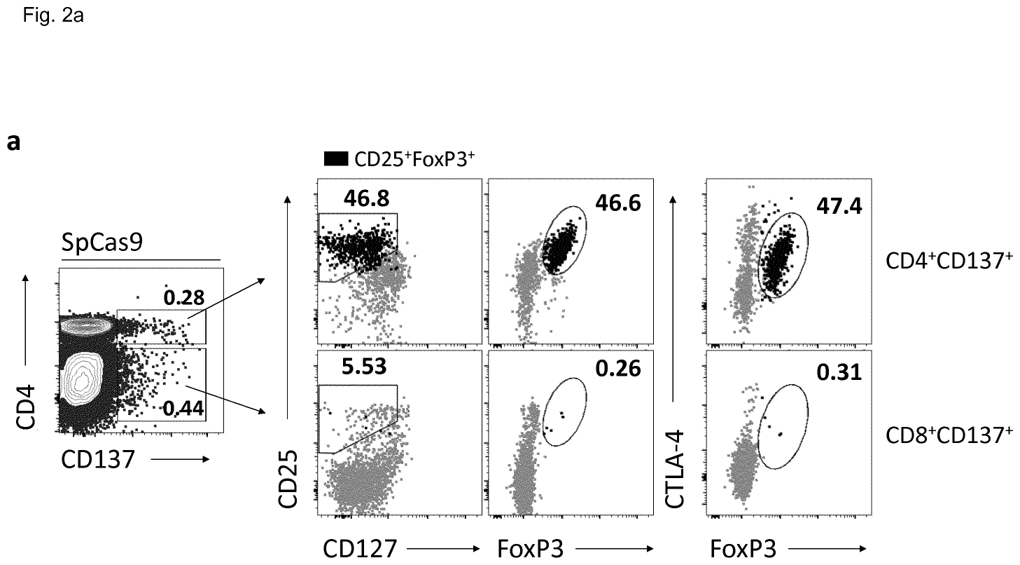

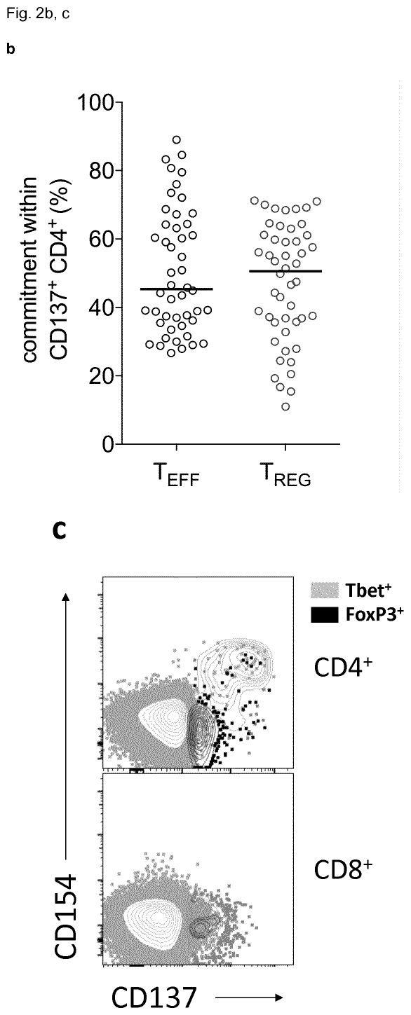

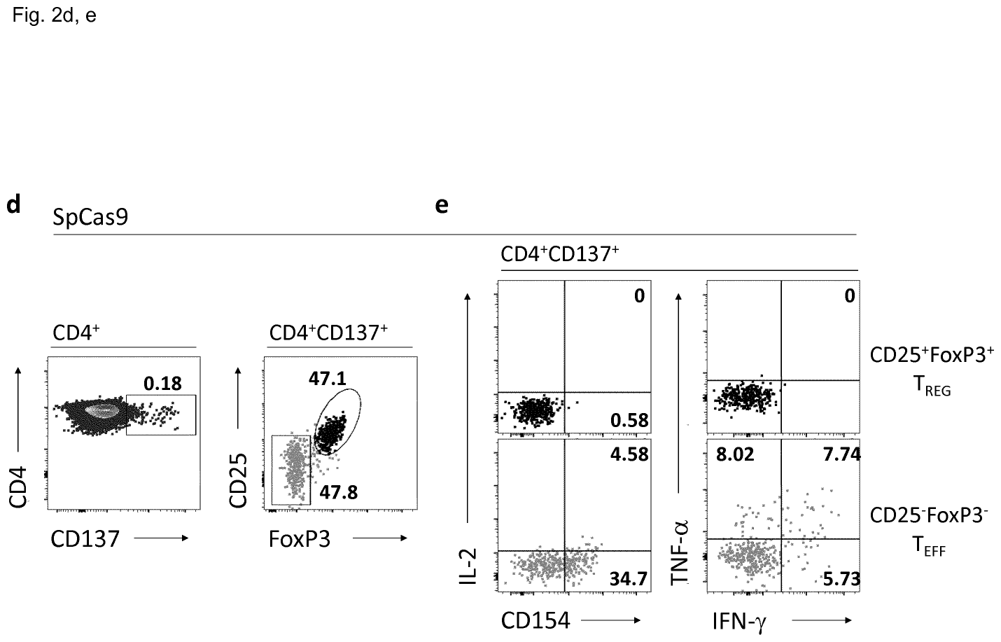

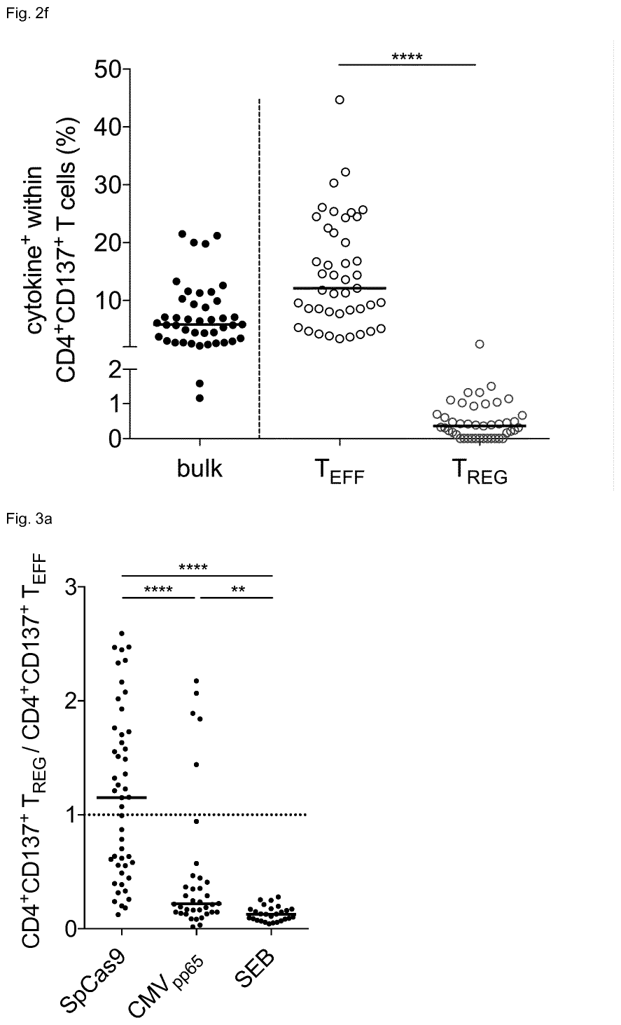

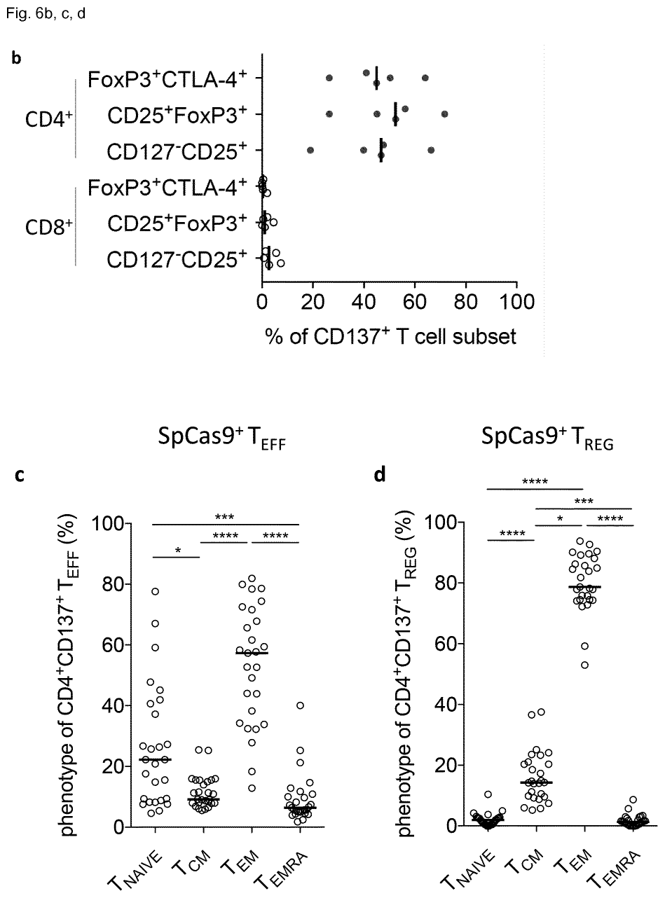

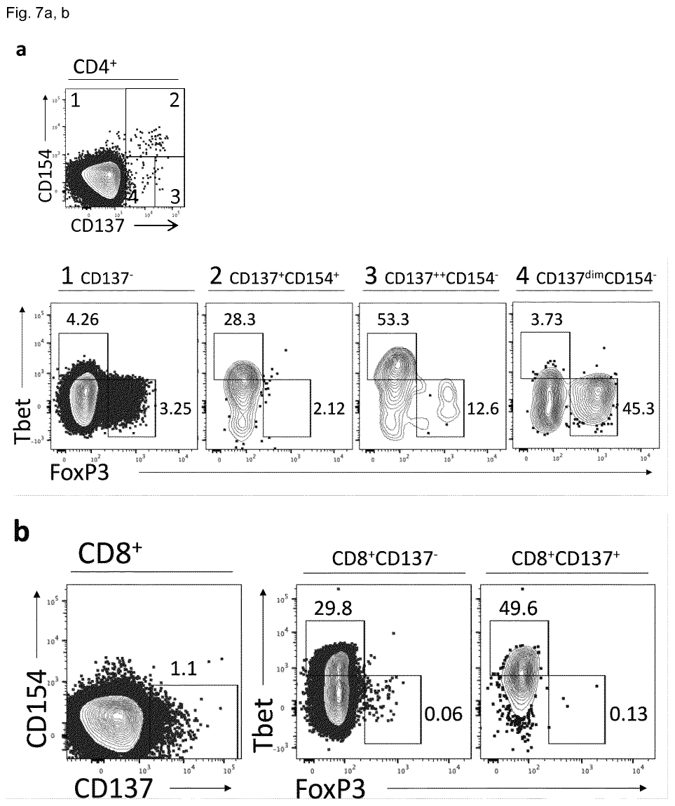

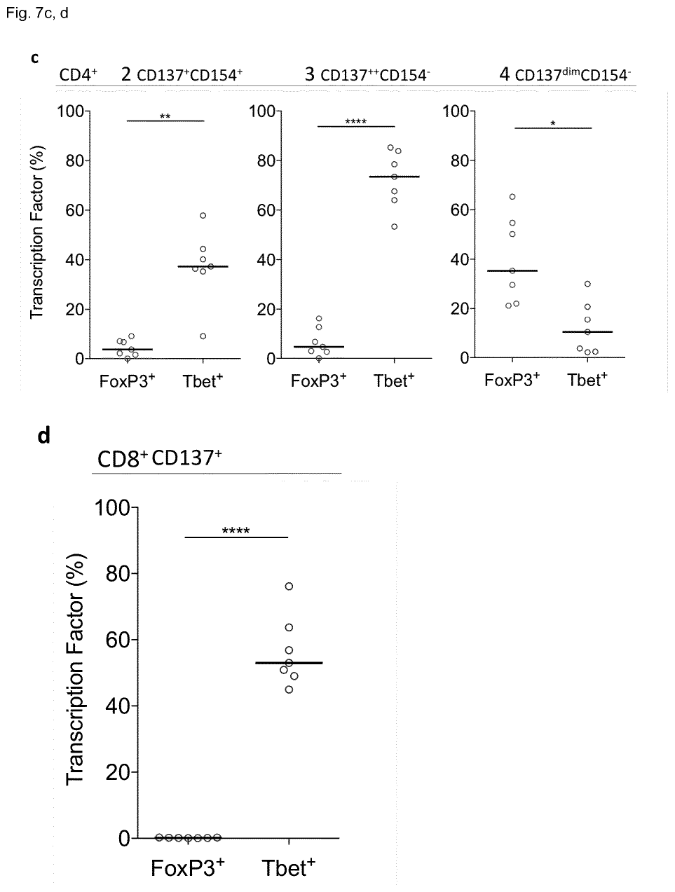

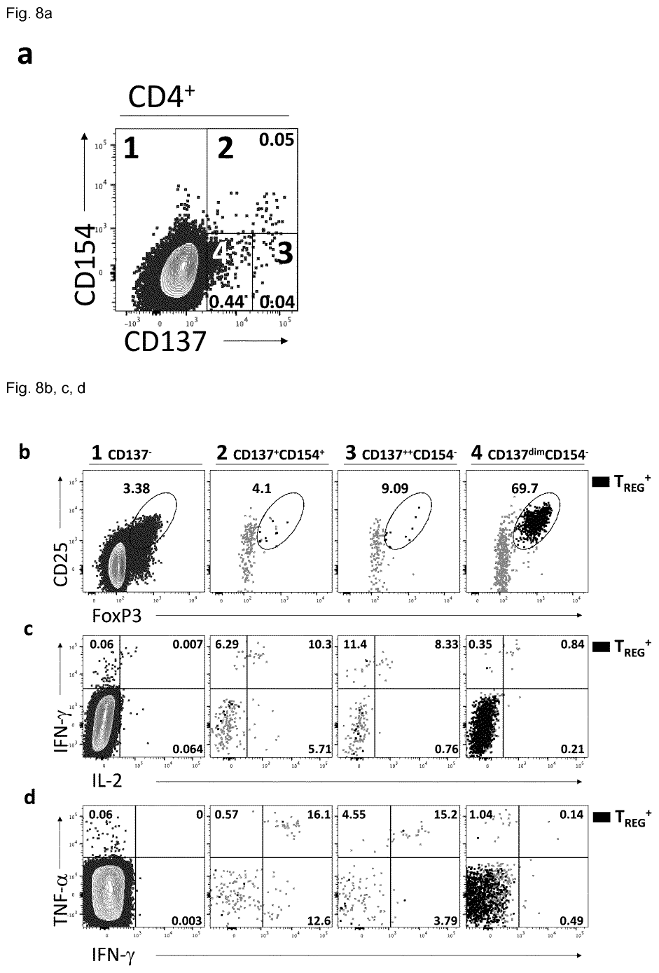

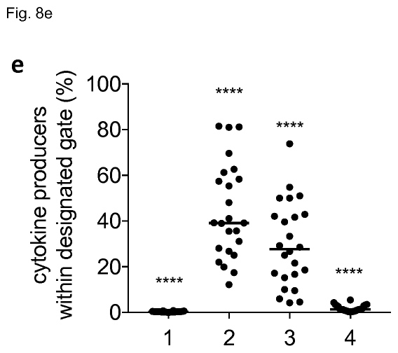

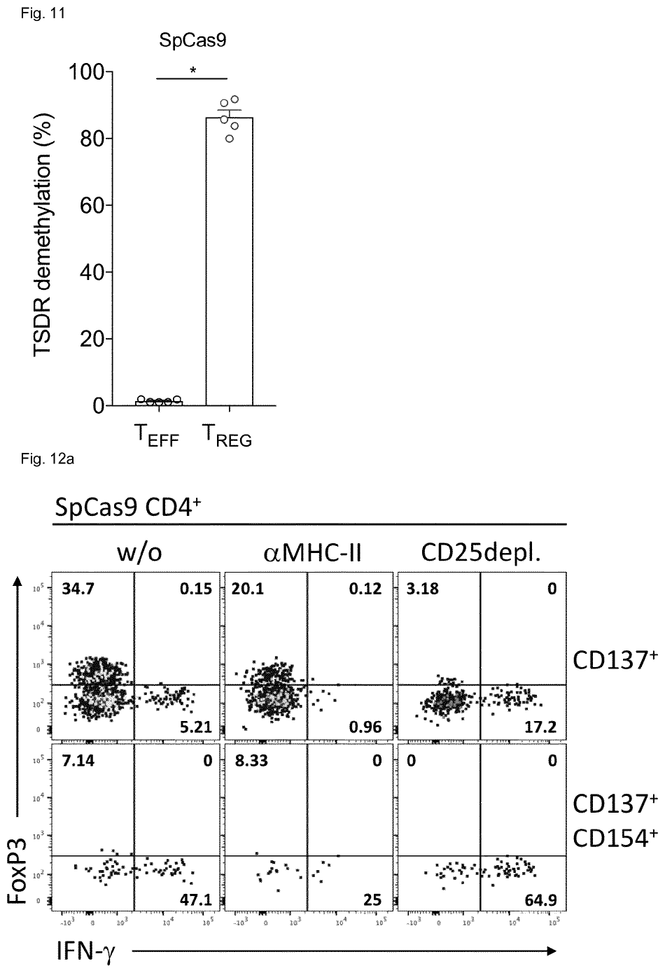

Commensal bacteria like S. pyogenes show repeated/continuous colonization on body surfaces. Recent studies indicate that continuous colonialization and repetitive exposure to environmental proteins or pathogens particularly at mucosal surfaces also induce T_{REG}. These T_{REG} are required to balance immune responses or even to maintain tolerance against innocuous environmental antigens. These findings expanded the significance of T_{REG} from controlling auto-reactivity towards a general role for protection against tissue-damaging inflammation. To determine the relative contribution of T_{REG} to the SpCas9-induced T cell response, the inventors performed intracellular staining for the T_{REG} lineage determining transcription factor FoxP3 in concert with CD25 surface expression (Sakaguchi, S. et al. 1995, J. Immunol. 155, 1151-64; Hori, S. et al. 2003, Science 299, 1057-1061). Further, the inventors combined those T_{REG} defining markers with activation marker and cytokine profiling following SpCas9 whole protein stimulation (FIG. 2a, d, FIG. 6). Intriguingly, the inventors found excessive frequencies of T_{REG} within SpCas9-reactive CD4⁺ CD137⁺ T cells ranging from 11% to 73.5% of total CD4+ response (FIG. 2a, b). T_{REG} identity was confirmed through additional phenotypic marker combinations like FoxP⁺CTLA-4⁺ or CD127^{low}CD25^{high} (FIG. 2a, FIG. 6a, b) and epigenetic analysis of the T_{REG}-specific demethylation region (average TSDR demethylation: T_{REG} 86.3%; T_{EFF} 1.39%; n=5, FIG. 11) (Wing, K. et al. 2008, Science 322, 271-275; Wieczorek, G. et al. 2009, Cancer Res. 69, 599-608). Further investigation of the SpCas9-induced T cell activation revealed distinct T cell lineage determining transcription factor profiles. CD4⁺FoxP3⁺ T_{REG} were exclusively found within the CD137^{dim}CD154⁻ population, while CD4⁺Tbet⁺ T_{EFF} comprised both CD137⁺CD154⁺ and CD137^{high} SpCas9-responsive populations (FIG. 2c, FIG. 7). Functionally, T_{REG} did not contribute to SpCas9-induced effector cytokine production (FIG. 2d-f, FIG. 8) but displayed a memory phenotype (FIG. 6d).

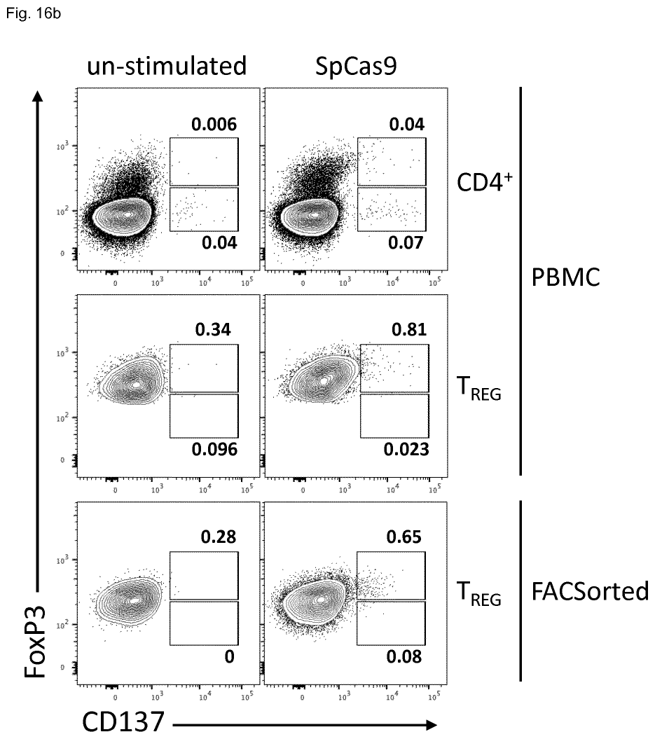

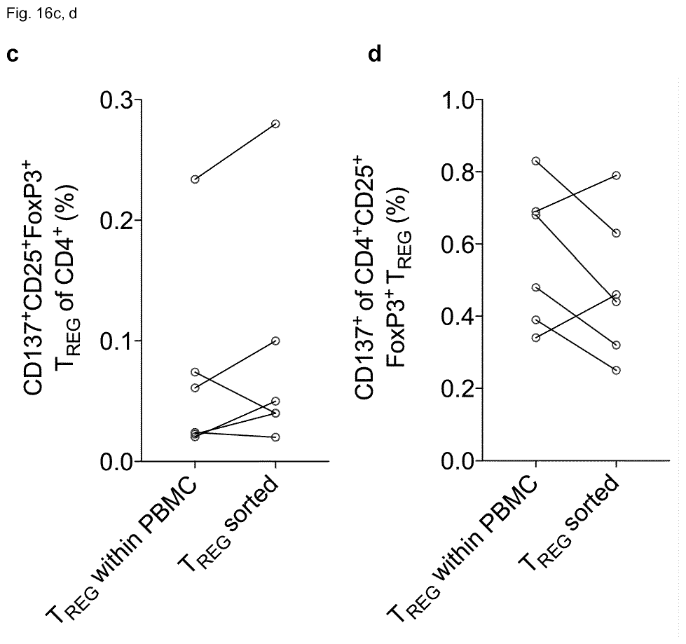

The inventors excluded Cas9-mediated T_{reg} activation is caused by bystander activation through effector T cells (FIG. 16). Compared to SpCas9-stimulated PBMCs, stimulation of bulk pre-enriched T_{reg} cells revealed no differences in SpCas9-reactive T_{reg} frequencies, excluding unspecific activation of bystander T_{reg} cells through SpCas9-reactive T_{eff} cells (FIG. 16 a-d).

Taken together, these findings demonstrate that SpCas9-specific T_{REG} are an inherent part of the physiological human SpCas9-specific T cell response.

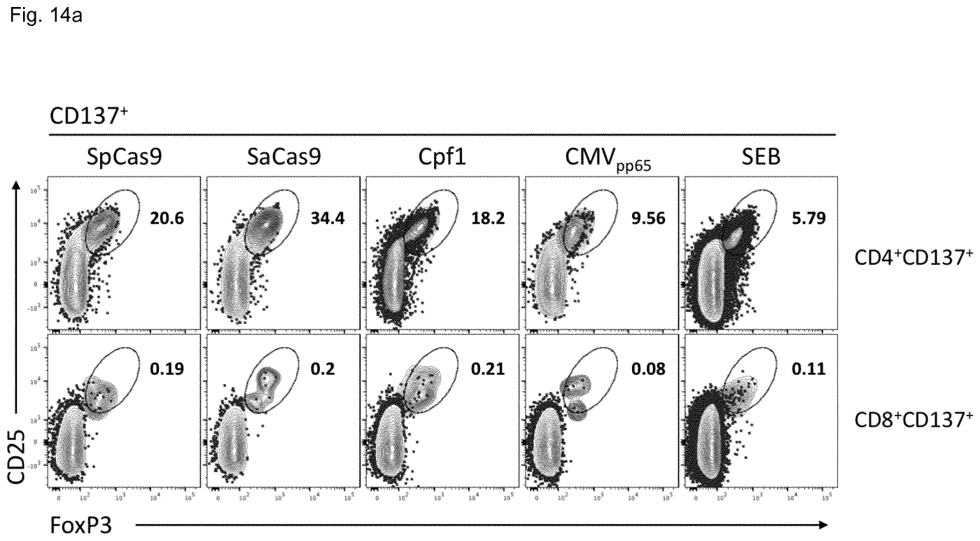

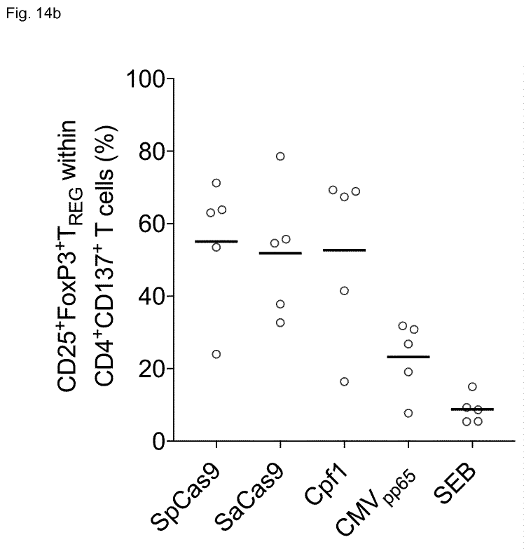

Although approximately 10% of all bacteria have class 2 CRISPR-Cas systems, only few have been adapted for gene editing. Strikingly, stimulation with other CRISPR-associated nuclease proteins from Acidaminococcus sp. Cas12a (also known as Cpf1) and Staphylococcus aureus Cas9 (SaCas9) yielded similar frequencies of activated T_{eff} and T_{reg} within the respective antigen response (FIG. 14 a,b). The SpCas9 protein has 26% shared protein sequence identity with its homolog SaCas9 and 38% with its homolog AsCpf1. Sequence homology between Cas proteins could lead to activation of T cells with specificity toward Cas epitopes that are shared between different bacterial species. Furthermore, S. aureus and Acidaminococcus sp. are common facultative pathogenic species; a separate immunization to their bacterial antigens may explain the observed T cell response. In particular, Acidaminococcus sp. may induce Treg cells as a critical regulator for immune tolerance in the gastrointestinal tract like other intestinal bacteria.

### Example 3: Relationship Between SpCas9-Specific T_{EFF} and T_{REG}

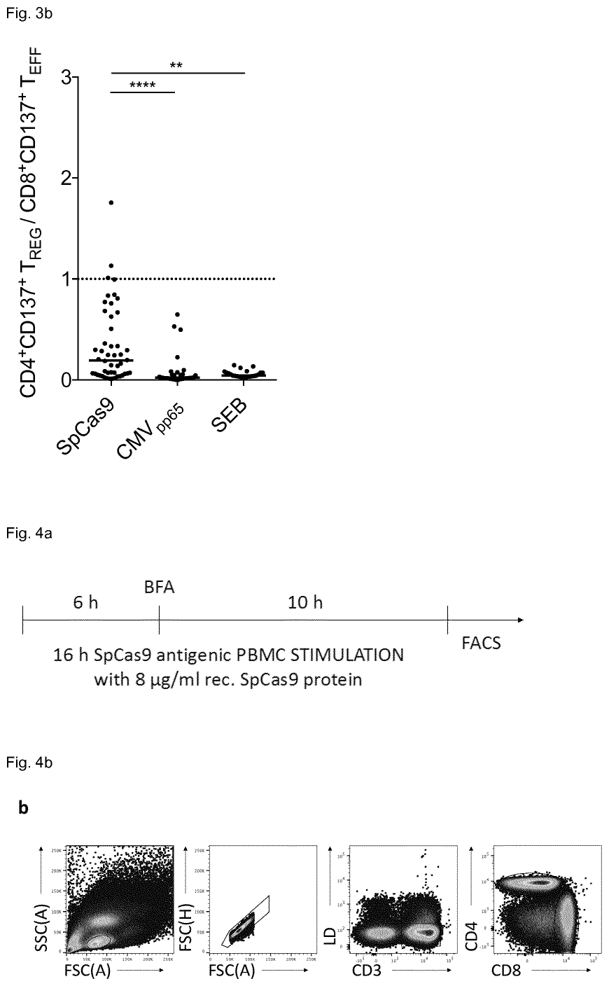

Next, the inventors investigated the individual relationship of T_{EFF} and their T_{REG} counterpart within the SpCas9-T cell response in comparison to an antiviral CMV and bacterial superantigen by relating the frequency of SpCas9, CMV phosphoprotein 65 (CMVₚₚ₆₅) and Staphylococcus Enterotoxin B (SEB)-activated T_{REG} to those of T_{EFF} within CD4⁺CD137⁺ and T_{EFF} within CD8⁺CD137⁺ antigen-reactive T cells. Remarkably, a balanced effector/regulatory T cell response to SpCas9 for both, CD4⁺ and CD8⁺, T cell compartments was found while response to CMVₚₚ₆₅ as well as SEB was dominated by T_{EFF} (FIG. 3a, b). Similarly, stimulations of PBMCs with other CRISPR-associated nucleases like SaCas9 and AsCpf1 showed similar results. (FIGS. 15 and 14)

A misbalanced Cas-reactive T_{REG}/T_{EFF} ratio may result in an overwhelming effector immune response to Cas following in vivo CRISPR/Cas9 gene editing.

### Example 4: Isolation and Expansion of SpCas9-Specific T_{EFF} and T_{REG}

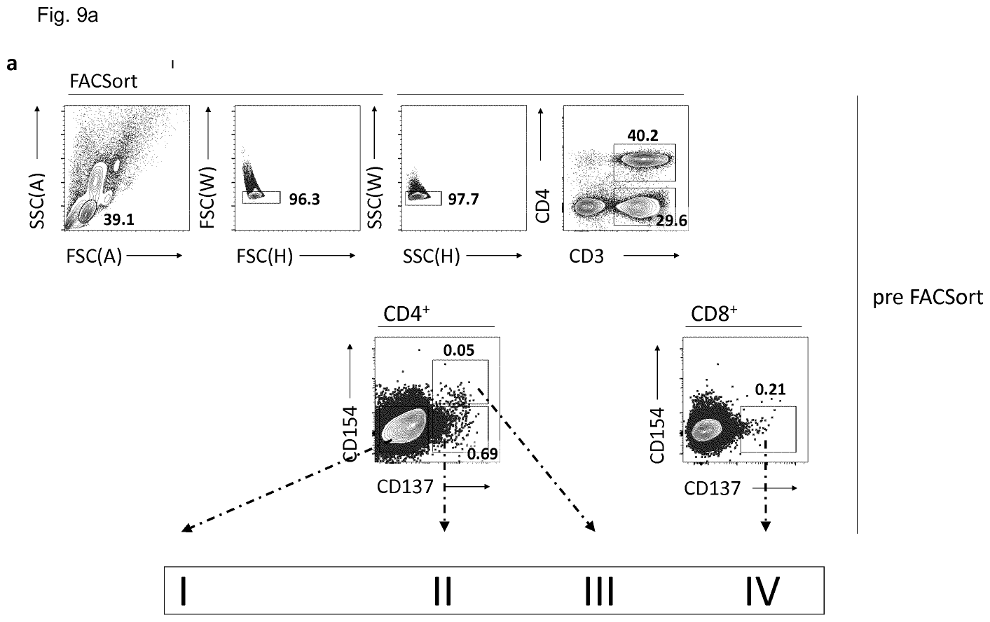

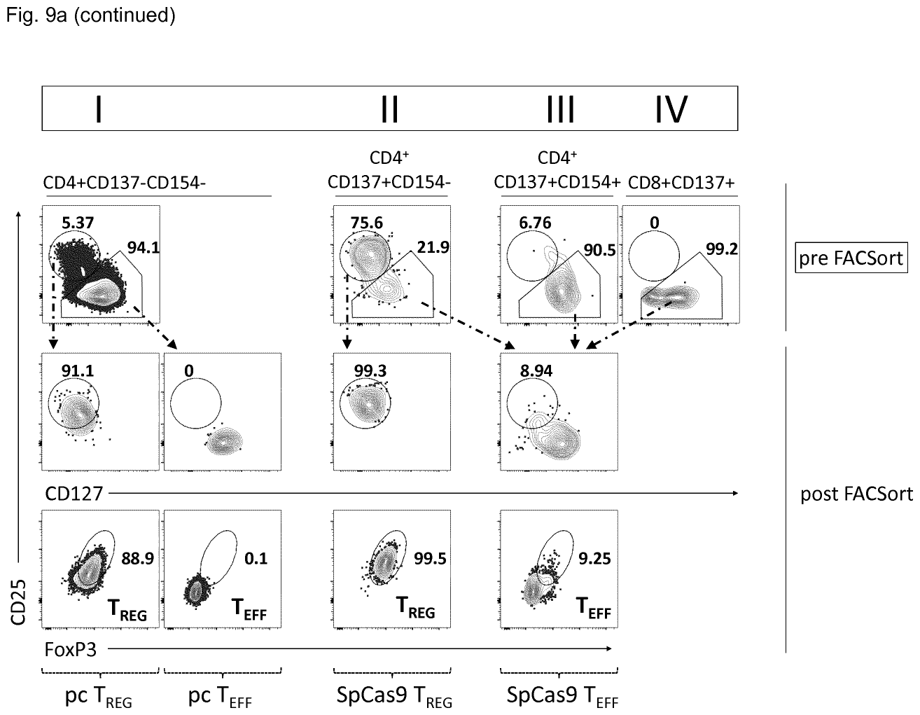

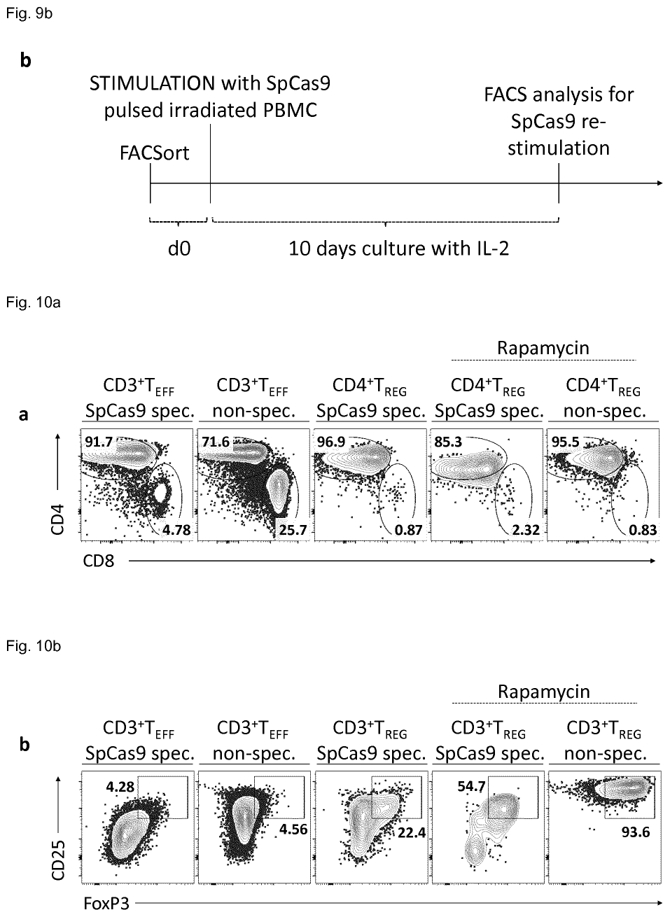

Several preclinical and first clinical data show that adoptively transferred T_{REG} are able to combat not only T cell priming but also overwhelming T_{EFF} response. Therefore, SpCas9-specific T_{REG} may have the potential to mitigate a SpCas9-directed T_{EFF} response. Having demonstrated that some individuals have a relatively low SpCas9-specific T_{REG}/T_{EFF} ratio, adoptive transfer of those cells would be an option. Therefore, enrichment and in vitro expansion of both SpCas9-specific T_{EFF} and T_{REG} were tested (FIG. 9).

Notably, most cells within the SpCas9-specific T_{REG} lines lost their T_{REG}-specific phenotype when cultured with IL-2, but were stabilized in the presence of the mTOR-inhibitor rapamycin, which is commonly used for expansion of thymic-derived naturally occurring T_{REG}.

### Example 5: SpCas9-Specific T_{EFF} Cell Lines Detect SpCas9 in Autologous Cells

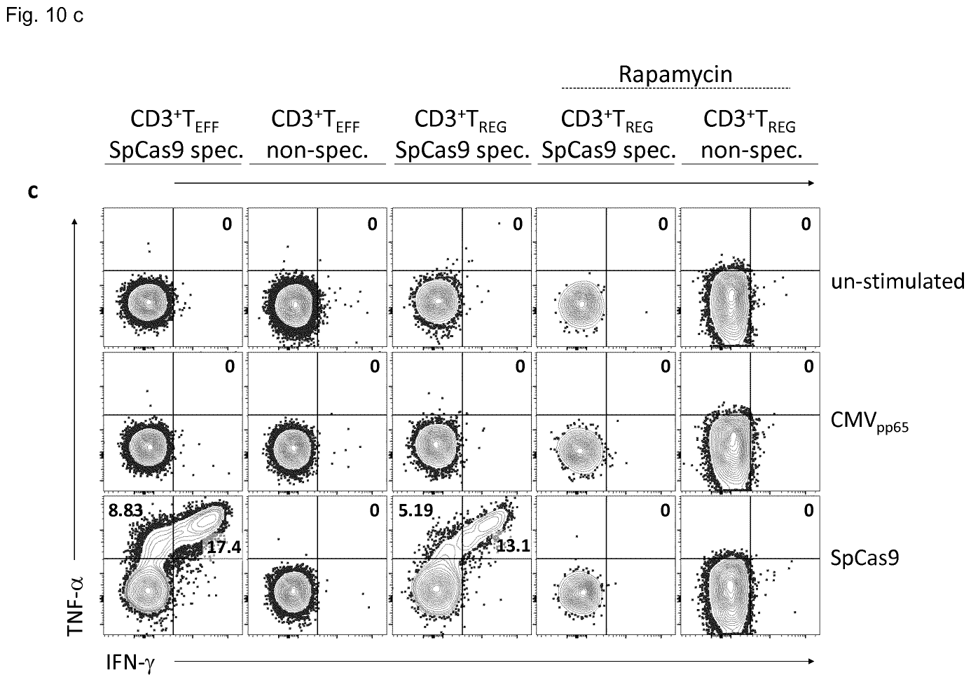

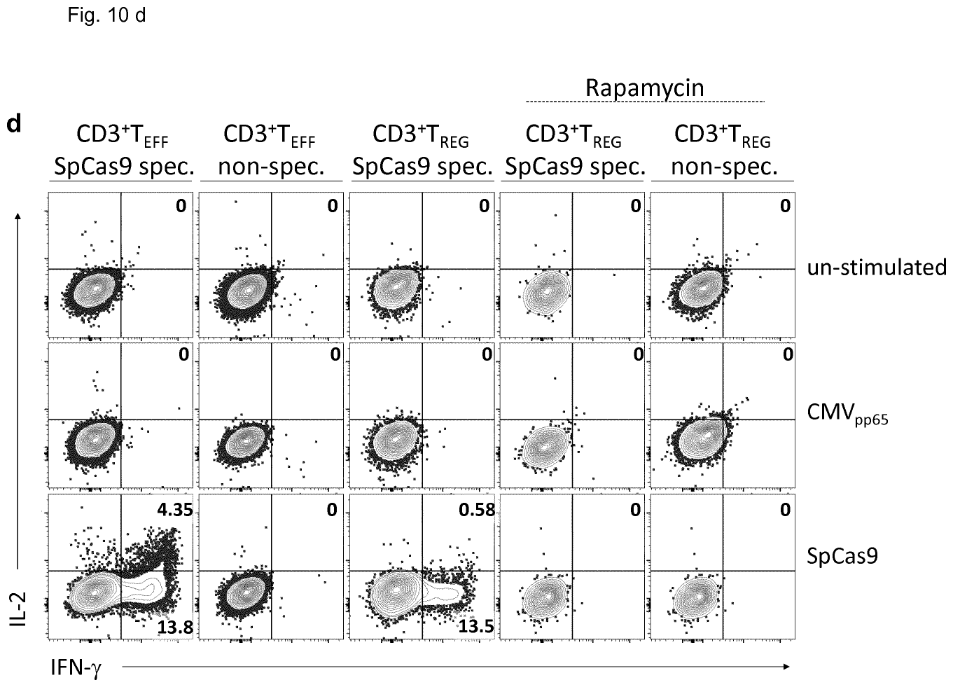

Technologies relying on ex vivo modification will not have a problem with immunogenicity because the gene-edited cells can be infused after complete degradation of the Cas9 protein. Unresponsiveness of autologous SpCas9-specific T_{EFF} lines to stimulation with CRISPR/Cas9-edited cell samples could be a release criterion for cell/tissue products in CRISPR/Cas9-related gene therapy (FIG. 10).

To examine their SpCas9-specific effector function, T_{EFF} lines were re-stimulated with SpCas9-loaded APCs after expansion and pronounced effector cytokine production was detected by intracellular flow cytometry. This indicates that APCs carry SpCas9-derived peptide antigens on their MHC molecules. (FIG. 10).

Furthermore, SpCas9-specific T_{EFF} cell lines had the capacity to lyse autologous target cells that endogenously express SpCas9 by forced overexpression through a DNA plasmid vector. (FIG. 13) Most in vivo gene therapeutic approaches using CRISPR-Cas9 aim to endogenously express SpCas9 and the respective single guide RNA within the target cell through viral or nonviral vectors. This experiment models the scenario of a gene therapeutic approach and indicates that

Cas-edited cells can be recognized and killed by the pre-primed and activated Cas-specific T cells. (FIG. 13)

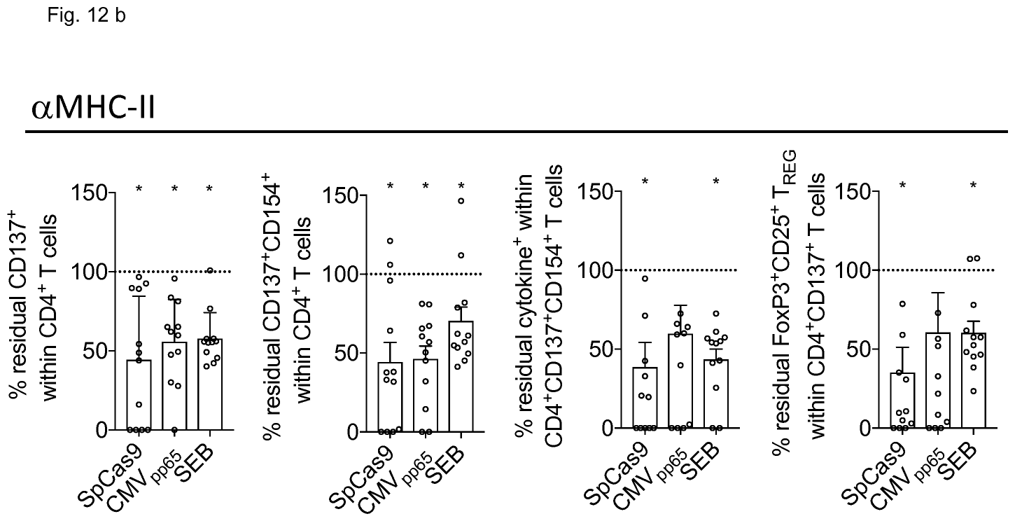

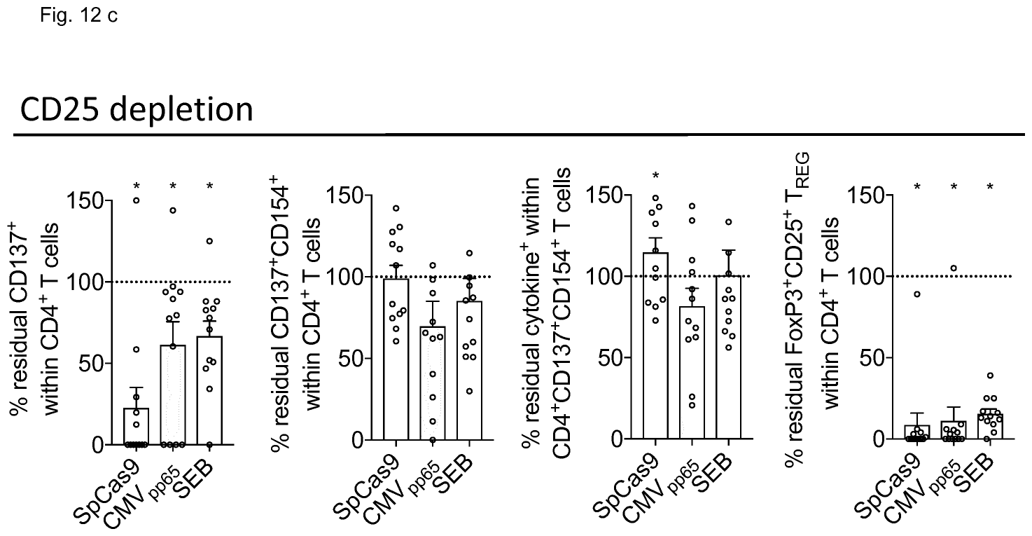

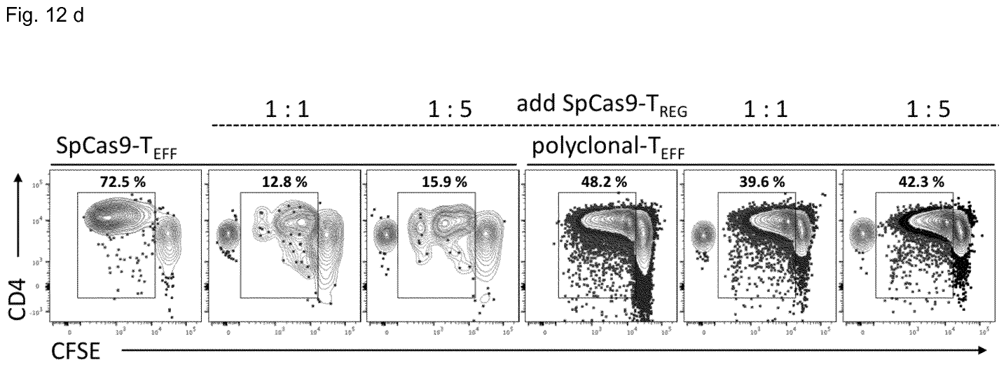

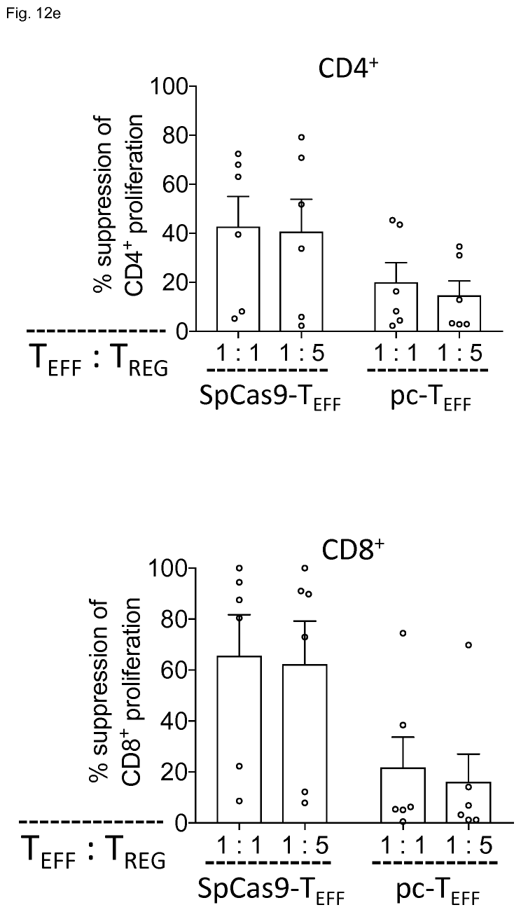

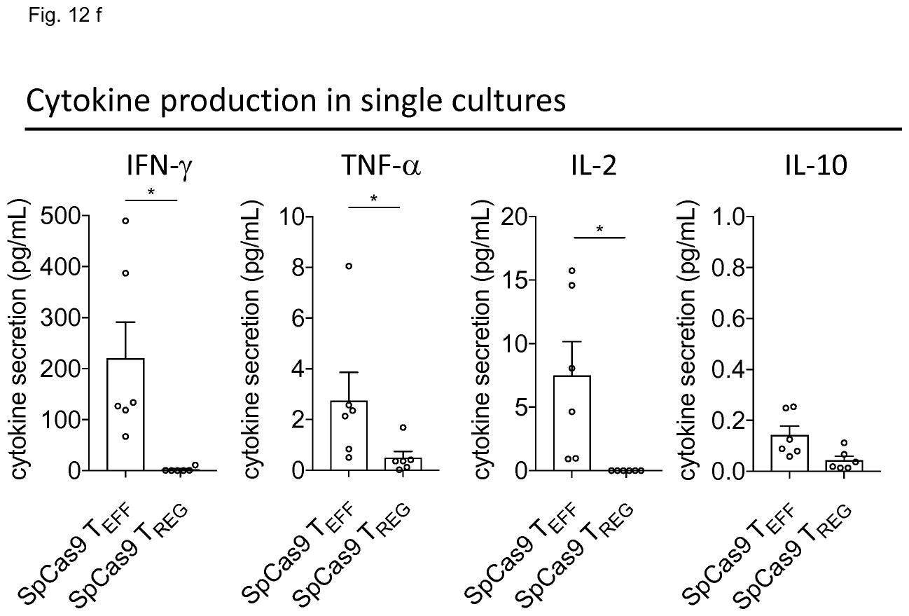

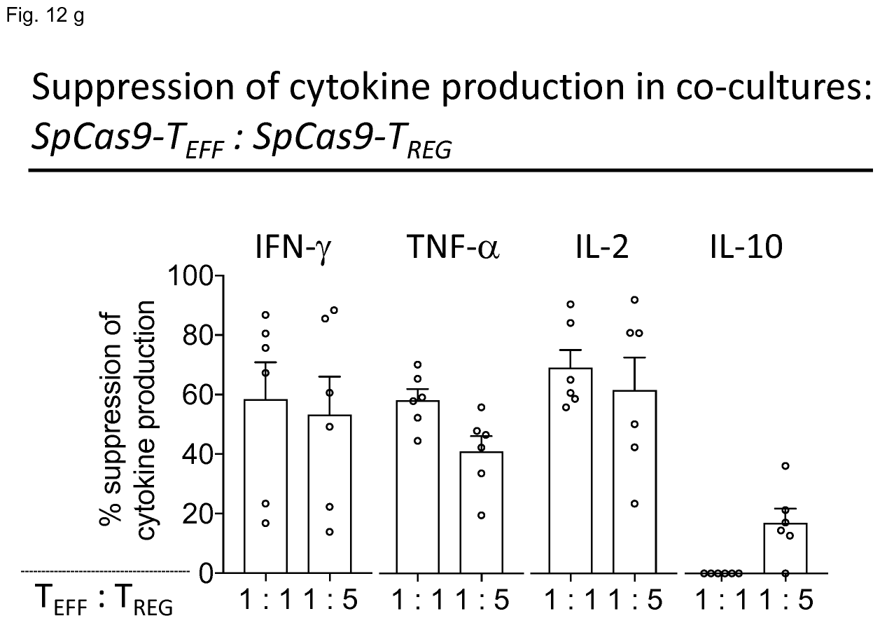

The inventors show, that autologous Cas-specific T_{EFF} cell lines can be generated from the peripheral blood from sensitized humans. To secure the complete degradation of Cas proteins after generation of a Cas-modified cell product, part of the cell product could be exposed to Cas-specific T_{EFF} lines. Then, apoptosis of the modified cell product (FIG. 13, VITAL-assay or similar) or production of effector cytokines could be detected within or on cells (intracellular flow as in FIG. 10, mRNA quantification or Elispot) or in supernatants by various methods (for example ELISA as in FIG. 12f, Western Blot, etc).

### Conclusion

In conclusion, the findings imply the requirement for controlling Cas-directed T_{EFF} response for successful CRISPR/Cas9 gene editing in vivo. The results emphasize the necessity of stringent immune monitoring of SpCas9-specific T cell responses, preceding and accompanying clinical trials employing Cas9-derived therapeutic approaches to identify potentially high-risk patients. Henceforth, misbalanced T_{REG}/T_{EFF} ratios and strong CD8⁺ T cell responses to SpCas9 may exclude patients for Cas9-associated gene-therapy.

### Example 6: SpCas9-Specific T_{REG} can Suppress SpCas9-Directed T_{EFF} Function

For in vivo application of CRISPR/Cas9, immunosuppressive treatment must be considered, especially if the control by T_{REG} is insufficient due to low T_{REG}/T_{EFF} ratio. Immunosuppressive drugs discussed for AAV-related gene therapy in naïve recipients, such as CTLA4-IgG and low dose prednisone, are inadequate to control a pre-existing T_{EFF} response. Adoptive transfer of SpCas9-specific T_{REG} should be considered as an approach to prevent hazardous inflammatory damage to CRISPR/Cas9-edited tissues and would circumvent the need for global immunosuppression.

As a proof of principle, the inventors tested whether SpCas9-directed T_{REG} cells could suppress T_{EFF} responses towards SpCas9. Hence, the enrichment strategies described in FIG. 9 were applied. Enriched SpCas9-reactive T_{REG} cells were able to inhibit the proliferation and cytokine production of SpCas9-activated T_{EFF} cells in proliferation suppression assays in vitro (FIG. 12). Natural occurring polyclonal thymic-derived T_{REG} cell lines inhibited Cas9-specific T_{EFF} less efficiently, showing an advantage of a Cas-specific T_{REG} cell product for clinical applications.

Our data indicate that endogenous SpCas9-specific T_{REG} cells can mitigate the activation, expansion, and function of SpCas9-specific T_{EFF}. Recent preclinical and first clinical data show that adoptively transferred T_{REG} cells can combat T cell priming but also overwhelming effector responses (Lei et al 2015, Front. Pharmacol. 6, 184; Chandran et al 2017 Am. J. Transplant. 17, 2945-2954).

### Materials and Methods

### Cell Preparation

Blood samples from healthy volunteers were collected after obtaining informed consent. PBMCs were separated from heparinized whole blood from healthy donors at different days (median age: 30, range: 18-57, 12 female/12 male) by lymphoprep density gradient centrifugation with a Biocoll-separating solution. PBMCs were cultured in complete medium, comprising VLE-RPMI 1640 medium supplemented with stable glutamine, 100 U/ml penicillin, 0.1 mg/ml streptomycin (all from Biochrom, Berlin, Germany) and 10% heat-inactivated FCS (PAA).

### Flow Cytometry Analysis

Freshly isolated PBMCs were stimulated in polystyrene round bottom tubes (Falcon, Corning) at 37° C. in humidified incubators and 5% CO₂ for 16 h with the following antigens: 8 μg/ml Streptococcus pyogenes (Sp) CRISPR associated protein 9 (Cas9) (SpCas9) (PNA Bio Inc., CA, USA), 1 μg/ml SEB (Sigma) and CMVₚₚ₆₅ overlapping peptide pool at 1 μg/ml (15mer, 11 aa overlap, JPT Peptide Technologies, Berlin, Germany). For functional and phenotypic characterisation, 5×10⁶ PBMC/1 ml complete medium were stimulated. For analysis of antigen-induced intracellular CD154 and CD137 expression and IFN-γ, TNF-α and IL-2 production, 2 μg/ml Brefeldin A (Sigma) were added. To allow for sufficient SpCas9 antigenic APC processing and presentation, Brefeldin A was added for the last 10 h of stimulation. After harvesting, extracellular T cell memory phenotype staining was performed using fluorescently conjugated monoclonal antibodies for CCR7 (PE, clone: G043H7), CD45RA (PE-Dazzle 594, clone: HI100) and CD45RO (BV785, clone: UCHL1) for 30 min at 4° C. In certain experiments CD25 (BD, APC, clone: 2A3), CD127 (Beckman Coulter, APC-Alexa Fluor 700, clone: R34.34) and CD152 (CTLA-4) (BD, PE-Cy5, clone: BN13) antibodies were used to define T_{REG} specific surface molecule expression. To exclude dead cells, LIVE/DEAD Fixable Blue Dead Stain dye (Invitrogen) was added. Subsequently, cells were fixed and permeabilised with FoxP3/Transcription factor staining buffer set (eBioscience) according to the manufacturers instructions. After washing, fixed cells were stained for 30 min at 4° C. with the following monoclonal antibodies: FoxP3 (Alexa Fluor 488, clone: 259D), CD3 (BV650, clone: OKT3), CD4 (PerCp-Cy5.5, clone: SK3) CD8 (BV570, clone: RPA-T8), CD137 (PE-Cy7, clone: 4B4-4), CD154 (BV711, clone 24-31), IFN-γ (BV605, clone 4S.B3), TNF-α (Alexa Fluor 700, clone: MAb11) and IL-2 (BV421, clone MQ1-17H12)).

Where indicated, PBMCs were depleted for CD4⁺ or CD25⁺ cells using MicroBeads (Miltenyi Biotech), following the manufacturer's instructions. For functional and phenotypic characterization, 5×10⁶ PBMCs per 1 ml complete medium were stimulated. Where indicated, 15 μg ml⁻¹ of MHC class II-blocking antibody (LEAF purified anti-human HLA-DR antibody; BioLegend) was applied during stimulation. In particular experiments, polyclonal T_{reg} cells were enriched in bulk by FACS, as described in the SpCas9-reactive T cell isolation section of the Methods, according to the cell surface expression of CD4⁺CD25⁺CD127⁻, rested overnight at 37° C. and 5% CO₂ in humidified incubators and subsequently stimulated with 5 μg ml⁻¹ SpCas9-pulsed monocytes (sorted according to the side scatter/forward scatter (FSC) profile) and B cells (sorted CD3⁻ fraction). Intracellular, T_{reg}-specific FOXP3 transcription factor staining was performed post-sorting. Post-sorting analysis of purified T_{reg} cells revealed purities >95%.

In particular experiments, antibodies for intracellular fluorescence staining of Tbet (Alexa Fluor 647, clone: 4610) and FoxP3 were used to define T cell lineage determining transcription factor expression levels. All antibodies were purchased from Biolegend, unless indicated otherwise.

Cells were analysed on a LSR-II Fortessa flow cytometer (BD Biosciences) and FlowJo Version 10 software (Tree Star). For ex vivo analysis, at least 1×10⁶ events were recorded. Lymphocytes were gated based on the FSC versus SSC profile and subsequently gated on FSC (height) versus FSC to exclude doublets. Unstimulated PBMC were used as controls and respective background responses have been subtracted from SpCas9 or CMVₚₚ₆₅-specific cytokine production (FIG. 1d). Negative values were set to zero.

### SpCas9-Specific T Cell Isolation and Expansion

Isolation: PBMCs were separated from 80 mL heparinized whole blood. PBMCs were washed twice with PBS and cultured for 16 h at 37° C. in humidified incubators and 5% CO₂ in the presence of 8 μg/ml SpCas9 whole protein and 1 μg/ml CD40-specific antibody (Miltenyi Biotech, HB 14) at cell concentrations of 1×10⁷ PBMCs per 2 mL VLE-RPMI 1640 medium with stable glutamine supplemented with 100 U/ml penicillin, 0.1 mg/ml streptomycin and 5% heat-inactivated human AB serum (PAA) in polystyrene flat bottom 24 well plates (Falcon, Corning). After stimulation, cells were washed with PBS (0.5% BSA) and stained for 10 minutes with BV650-conjugated CD3-specific antibody, PerCp-Cy5.5-conjugated CD4-specific antibody, APC-conjugated CD25-specific antibody, APC-Alexa Fluor 700-conjugated CD127-specific antibody (Beckman Coulter), PE-Cy7-conjugated CD137-specific antibody and BV711-conjugated CD154-specific antibody. SpCas9-specific T_{REG} (FIG. 9a: CD3⁺CD4⁺CD137⁺CD154⁻CD25^{high} CD127⁻) and SpCas9-specific T_{EFF} (FIG. 9a: CD3⁺CD137⁺CD154⁺CD25^{low}) were enriched by fluorescently activated cell sorting on a BD FACSAriaII SORP (BD Biosciences). In addition, polyclonal (pc) T_{REG} (FIG. 9a: CD3⁺CD4⁺CD137⁻CD154⁻CD25^{high} CD127⁻) and pc T_{EFF} (FIG. 9a: CD3⁺CD137⁺CD154⁺CD25^{low}) were enriched for non-specific expansion. Intracellular T_{REG}-specific FoxP3 transcription factor staining was performed post-sorting. Post-sorting analysis of purified subsets revealed greater than 90% purity.

Expansion: Isolated SpCas9-specific T_{EFF} and control pc T_{EFF} cells were cultured at 37° C. in humidified incubators and 5% CO₂ at a ratio of 1:50 with irradiated autologous PBMC (30 gy) in a 96-well plate (Falcon, Corning) with RPMI medium containing 5% human AB serum including 50 U/mL recombinant human (rh) IL-2 (Proleukin, Novartis). Isolated SpCas9-specific T_{REG} cells were cultured at 37° C. in humidified incubators and 5% CO₂ at a ratio of 1:50 with irradiated autologous PBMC (30 gy) in a 96-well plate with X-Vivo 15 Medium (Lonza) containing 5% human AB serum including 500 U/mL rh IL-2 in the presence or absence of 100 nM rapamycin (Pfizer). Non-specific pc T_{REG} were activated for polyclonal expansion applying the T_{REG} expansion kit according to the manufacturers instructions (T_{REG}: bead ratio of 1:1; CD3/CD28 MACSiBead particles, Miltenyi Biotech, Germany) and cultured in X-Vivo 15 Medium in the presence of 100 nM rapamycin. A minimum of 10⁴SpCas9-specific CD137⁺CD154⁻ T_{REG} cells was isolated, which could be expanded in vitro to at least 10⁵ cells within 10 days. Medium and cytokines were added every other day or when cells were split during expansion.

### In Vitro Restimulation of Ex Vivo Isolated and Expanded SpCas9-Specific T Cells

Cultured SpCas9-specific T_{EFF} and T_{REG} were analysed at day 10 for expression of effector molecules in response to stimulation with SpCas9 whole protein-loaded autologous monocyte-derived dendritic cells (moDCs). CD14⁺ monocytes were enriched from PBMCs by magnetically activated cell sorting (MACS, Miltenyi Biotech). Subsequently, CD14⁺ cells were cultured for 5 days in 1,000 IU/mL rhGM-CSF (Cellgenix) and 400 IU/mL rhIL-4 (Cellgenix). Then, fresh medium with 1,000 IU/ml TNF-α (Cellgenix) was supplied. During 48 h of TNF-α induced maturation of autologous moDCs 4 μg/ml SpCas9 was added. Expanded T cell subsets were re-stimulated with either SpCas9-pulsed, 1 μg/ml CMVₚₚ₆₅ overlapping peptide pool-pulsed or un-pulsed autologous moDCs for 6 h at a ratio of 10:1. 2 μg/ml Brefeldin A was added for the last 5 h of stimulation. Following stimulation, the expression of CD3, CD4, CD8, CD25, intracellular IFN-γ, TNF-α and IL-2, and intra-nuclear FoxP3 was analysed, and the cells were treated for flow cytometric readout as described above. Cells were stained with BV650-conjugated CD3-specific antibody, PerCp-Cy5.5-conjugated CD4-specific antibody, BV570-conjugated CD8-specific antibody, APC-conjugated CD25-specific antibody, BV605 conjugated IFN-γ-specific antibody, Alexa Fluor 700 conjugated TNF-α-specific antibody and BV421-conjugated IL-2-specific antibody.

### TSDR—Methylation Analysis

DNA methylation analysis of the T_{REG}-specific demethylation region (TSDR) was performed as previously described (Wieczorek, G. et al. 2009, Cancer Res. 69, 599-608). Briefly, bisulfite-modified genomic DNA (Quick-DNA Miniprep Plus Kit, Zymo Research, Irvine, USA; EpiTect Bilsulfite Kit, Qiagen, Hilden, Germany) was used in a real-time polymerase chain reaction for FoxP3 TSDR quantification. A minimum of 40 ng genomic DNA or a respective amount of plasmid standard was used in addition to 10 μl FastStart Universal Probe Master (Roche Diagnostics, Mannheim, Germany), 50 ng/μl Lambda DNA (New England Biolabs, Frankfurt, Germany), 5 pmol/μl methylation or nonmethylation-specific probe, 30 pmol/μl methylation or nonmethylation-specific primers (both from Epiontis, Berlin, Germany) in 20 μl total reaction volume. The samples were analysed in triplicate on an ABI 7500 cycler (Life Technologies Ltd, Carlsbad, USA).

### Proliferation Assay

PBMCs were labeled with 10 μM carboxyfluorescein succinimidyl ester (CFSE; Thermo Fisher Scientific), activated with SpCas9 whole protein, and cultured in complete medium for 5 d in the presence or absence of MHC class II-blocking antibody (MHC class II: HLA-DR). The frequencies of T cell proliferation (CFSE dilution) were assessed by flow cytometry following 5 d of culture.

### Proliferation Suppression Assay

SpCas9-reactive T_{reg}, SpCas9-reactive T_{eff}, and polyclonal T_{eff} cells were enriched as described in the SpCas9-reactive T cell isolation section. T_{eff} were labeled with 10 μM CFSE; Molecular Probes). CFSE-labeled SpCas9-reactive T_{eff} or polyclonal T_{eff} cells were cultured in complete medium alone or with autologous SpCas9-reactive T_{reg} at T_{eff}/T_{reg} ratios of 1:1 and 5:1. Polyclonal T_{eff} were stimulated with anti-CD3/CD28-coated microbeads (T_{reg}suppression inspector; Miltenyi Biotech) at a cell per bead ratio of 1:1 adjusted to the total cell number per well and incubated at 37° C. for 96 h. SpCas9-reactive T_{eff} were activated before sorting with no further stimulation and incubated at 37° C. for 96 h. Thereafter, cells were stained with CD3 (BV650, clone OKT3) and CD4 (PerCP-Cy5.5, clone SK3), all sourced from BioLegend. Dead cells were excluded using the LIVE/DEAD Fixable Aqua Dead Cell Stain Kit (Invitrogen). Proliferation was assessed by CFSE dilution; the percentage suppression of proliferation was calculated by relating the percentage of proliferating T_{eff} cells in the presence and absence of T_{reg}, respectively.

### Allocation and Culture of LCLs

SpCas9-reactive T lymphocytes were analyzed for effector functions by their ability to recognize SpCas9-transfected target cells, that is, autologous LCLs transformed with B95-8 Epstein-Barr virus as described previously (Heslop et al, 1996, Nature Medicine 2, 551-555 and Moosmann et al., 2002, Blood 100(5), 1755-64). Autologous primary Epstein-Barr virus-transformed LCLs were cultured in very-low-endotoxin RPMI 1640 medium supplemented with stable glutamine, 100 U ml⁻¹ penicillin, 0.1 mg ml⁻¹ streptomycin (all from Biochrom) and 10% heat-inactivated fetal bovine serum (PAA Laboratories).

### Transfection of Primary LCLs (Autologous Cells for Forced Cas9 Expression)

To determine the immunogenicity of endogenously expressed SpCas9, autologous LCLs were transfected with a DNA plasmid vector containing an expression cassette for SpCas9; 24-36 h before transfection, LCLs were seeded at a concentration between 2.5 and 5.0×10⁵ cells per ml of antibiotic-free cell culture medium. For transfection, LCLs were collected and washed twice with PBS. Cell pellets were resuspended at 1×10⁷ cells per ml Buffer R and supplemented with either pMAX-GFP (Lonza) with a final concentration of 25 μg ml⁻¹ as control or a SpCas9-expressing plasmid (PX458) with a final concentration of 100 μg ml⁻¹. pSpCas9(BB)-2A-GFP (PX458) was kindly provided by F. Zhang (Addgene). The PX458 plasmid contains a fusion protein of the S. pyogenes Cas9 nuclease and the GFP connected by the self-cleaving peptide P2A. After protein translation, P2A leads to the separation of single SpCas9 and a GFP protein, respectively. The inventors used a modified PX458 plasmid containing a single guide RNA targeting the hAAVS1 locus42, generously provided by A.-F. Hennig and U. Kornak. Transfection of LCLs was performed using 10 μl tips of the Neon Transfection System (Thermo Fisher Scientific) by electroporation with 3 pulses at 1,600 V for 10 ms. After electroporation, LCLs were directly transferred to prewarmed antibiotic-free medium and rested for 24 h before performing the cytotoxicity assays.

### Assessment of Cytotoxic Activity: VITAL Assay

A modified VITAL assay was used for cytotoxicity testing as described previously (Hammoud et al., 2013, Journal of Immunotherapy 36(2), 93-101). Briefly, transfected LCLs expressing SpCas9 and GFP (LCLs-SpCas9+GFP+) served as SpCas9-positive target cells for T cells and LCLs expressing GFP alone (LCLs-GFP⁺) served as SpCas9-negative target cells for T cells to exclude unspecific killing due to DNA plasmid electroporation and GFP expression. As internal controls, unmodified LCLs were labeled with 5 μM N,N-dimethyldodecylamine N-oxide (Invitrogen).

### T Cell Expansion

Isolated SpCas9-reactive T_{eff} cells were cultured in a U-bottom 96-well plate (Falcon; Corning) with RPMI medium containing 5% human AB serum including recombinant human IL-7 and IL-15 each at 10 ng ml⁻¹ (CellGenix) at 37° C. and 5% CO₂ in humidified incubators for 3 d. Isolated SpCas9-reactive T_{reg} cells were cultured in a U-bottom 96-well plate with RPMI medium containing 5% human AB serum including 500 IU ml⁻¹ recombinant human IL-2 (Proleukin; Novartis) at 37° C. and 5% CO₂ in humidified incubators for 3 d.

### Setting Up the Cytotoxicity Assay

Target and nontarget LCLs were cocultured for 16 h with SpCas9-reactive T cell/target cell ratios of 10:1, 1:1, and 1:10 (for electroporation; see the Transfection of primary LCLs section of the Methods). Samples without T cells, containing only targets and nontargets (LCL-SpCas9⁺GFP⁺/LCL-GFP⁺ and N,N-dimethyldodecylamine N-oxide-labeled unmodified LCLs), served as reference controls. After coculture, the cells were analyzed using the LSR-II Fortessa flow cytometer (BD Biosciences). Dead cells were excluded using the LIVE/DEAD Fixable Blue Dead Cell Stain dye (Thermo Fisher Scientific). The mean percentage survival of LCL-SpCas9⁺GFP⁺ target cells or LCL-GFP⁺ cells was calculated relative to the N,N-dimethyldodecylamine N-oxide-labeled unmodified LCL controls. Subsequently, the percentage of specific target cell lysis was calculated, comparing the mean percent survival of targets in cultures containing defined numbers of T_{eff} cells and the conditions without T cells.

### Statistical Analysis and Calculations

Graph Pad Prism version 7 was used for generation of graphs and statistical analysis. To test for normal Gaussian distribution Kolmogorov-Smirnov test, D'Agostino & Pearson normality test and Shapiro-Wilk normality test were performed. In two data set comparisons, if data were normally distributed Student's paired t test was employed for analysis. If data were not normally distributed Wilcoxon's matched pairs test was applied. All tests were two-tailed. Where more than two paired data sets were compared, one way ANOVA was employed for normally distributed samples and Friedman's test was used for not normally distributed samples. For comparison of more than two unpaired not normally distributed data sets, Kruskal-Wallis' test was applied. To exactly identify significant differences in not normally distributed data sets Dunn's multiple comparison test was used as post-test and the post-test employed for normally distributed samples was Tukey's multiple comparison test. Correlation analysis was assessed by Pearson's correlation coefficients for normally distributed data or non-parametric Spearman's rank correlation for not normally distributed data. The regression line was inserted based on linear regression analysis. Probability (p) values of ≤0.05 were considered statistically significant and significance is denoted as follows: *=p≤0.05; **=p≤0.01; ***=p≤0.001; ****=p≤0.0001.

1. A method for determining a T cell mediated immunity towards a CRISPR associated protein, particularly towards Cas9 or Cas12, more particularly towards SpCas9, or towards a homologue of such CRISPR associated protein, comprising the steps of
providing a cell preparation comprising T cells obtained from a patient;
in a stimulation step, contacting said cell preparation with
an isolated CRISPR associated protein polypeptide, particularly a Cas9 or Cas12 polypeptide, more particularly a SpCas9 polypeptide, or a homologue of such CRISPR associated protein, or
a plurality of peptides, wherein said plurality of peptides represents the amino acid sequence of said CRISPR associated protein polypeptide, particularly said Cas9 or Cas12 polypeptide, more particularly of said SpCas9 polypeptide, or said homologue thereof, or
a cell comprising a CRISPR associated protein polypeptide, particularly Cas9 or Cas12, more particularly SpCas9, or a homologue of such CRISPR associated protein,
in the presence of antigen presenting cells, particularly in the presence of autologous peripheral blood mononuclear cells,
providing activated T cells;
optionally, adding an inhibitor of intracellular protein transport, particularly Brefeldin A and/or Monensin, to said cell preparation during the last part of said stimulation step;
in a detection step, detecting one or more subpopulations of said activated T cells by
contacting said activated T cells with a set of molecular probes specific to activated regulatory T cells, and/or
contacting said activated T cells with a set of molecular probes specific to activated effector T cells,
providing marked activated regulatory T cells and/or marked activated effector T cells;
in a quantification step, determining a number of said marked activated regulatory T cells and a number of said marked activated effector T cells, and optionally, a ratio (T_{REG}/T_{EFF}) of the number of said marked activated regulatory T cells to said marked activated effector T cells.

2. A method for determining a nucleic acid sequence encoding a T cell receptor molecule capable of specifically recognizing an HLA-presented antigen derived from a CRISPR associated protein, particularly derived from a Cas9 or Cas12 protein, more particularly derived from an SpCas9 protein, or from a homologue of such CRISPR associated protein, comprising the steps of
providing a cell preparation comprising T cells obtained from a patient;
in a stimulation step, contacting said cell preparation with
an isolated CRISPR associated protein polypeptide, particularly a Cas9 or Cas12 polypeptide, more particularly an SpCas9 polypeptide, or a homologue of such CRISPR associated protein, or
a plurality of peptides, wherein said plurality of peptides represents the amino acid sequence of said CRISPR associated protein polypeptide, particularly said Cas9 or Cas12 polypeptide, more particularly said SpCas9 polypeptide, or said homologue thereof, or
a cell comprising a CRISPR associated protein polypeptide, particularly Cas9 or Cas12, more particularly SpCas9, or a homologue of such CRISPR associated protein,
in the presence of antigen presenting cells, particularly in the presence of autologous peripheral blood mononuclear cells,
providing activated T cells;
optionally, adding an inhibitor of intracellular protein transport, particularly Brefeldin A and/or Monensin, to said cell preparation during the last part of said stimulation step;
in an isolation step, isolating one or more subpopulations of said activated T cells by
contacting said activated T cells with a set of molecular probes specific to activated regulatory T cells, and/or
contacting said activated T cells with a set of molecular probes specific to activated effector T cells,
and removing a population of CRISPR specific regulatory T cells and/or CRISPR specific activated effector T cells from said preparation;
in a sequence determination step, determining one or more nucleic acid sequences encoding a CRISPR specific T cell receptor molecule comprised in said population of CRISPR specific T cells.

3. The method according to claim 1 or 2, wherein in the detection step of claim 1 or in the isolation step of claim 2, a cell is assigned an activated regulatory T cell that is
positive for CD3, CD4, CD137 and CD25, wherein CD25 is highly expressed, and negative for CD154, and
positive for FoxP3 and/or positive helios and/or negative for CD127,
and optionally, positive for any one of CD69, CD71, CD103, CD134, GARP, HLA-DR, IFNγ, IL-10, KLRG1, LAP, SATB1, TGFβ or TNFα.

4. The method according to any one of the preceding claims, wherein in the detection step of claim 1 or in the isolation step of claim 2, said set of molecular probes specific to activated effector T cells comprises
ligands specific to CD3, CD4, CD137 and CD154, and optionally, one or more ligands specific to any one of CD69, CD71, CD80, CD86, CD107a, CD134, Granzyme B, HLA-DR, IFNγ, IL-2, KLRG1, Perforin or TNFα; and/or
ligands specific to CD3, CD8 and CD137, and optionally, one or more ligands specific to any one of CD69, CD71, CD80, CD86, CD107a, CD134, Granzyme B, HLA-DR, IFNγ, IL-2, KLRG1, Perforin or TNFα; and/or
ligands specific to CD3, CD4 and CD137 and one or more ligands specific to CD25, FoxP3 and helios, and optionally, one or more ligands specific to any one of CD69, CD71, CD80, CD86, CD107a, CD134, Granzyme B, HLA-DR, IFNγ, IL-2, KLRG1, Perforin or TNFα;
particularly wherein in said isolation step or said detection step, a cell is assigned an activated effector T cell that is positive for CD3 and CD137, and
positive for CD4 and CD154 or
positive for CD8 or
positive for CD4 and
positive for CD25, wherein CD25 is lowly expressed, and/or
negative for FoxP3 or helios;
and optionally, positive for any one of CD69, CD71, CD80, CD86, CD107a, CD134, Granzyme B, HLA-DR, IFNγ, IL-2, KLRG1, Perforin or TNFα.

5. The method according to any one of claim 2, 3 or 4, wherein the expression of MHC-II isotypes is determined for said patient, and said sequences encoding a CRISPR specific T cell receptor molecule are assigned to an MHC-II isotype group.

6. The method according to any one of claims 2 to 5, wherein said method is repeated for a plurality of patients characterized by a shared MHC-II haplotype, and CRISPR specific T cell receptor molecules shared by a significant number of said patients are assigned to an MHC-II matching group.

7. The method according to any one of claims 2 to 6, wherein only CRISPR specific regulatory T cells are isolated, and nucleic acids encoding CRISPR specific regulatory T cell receptor molecules are determined or wherein only CRISPR specific effector T cells are isolated, and nucleic acids encoding CRISPR specific effector T cell receptor molecules are determined.

8. The method according to any one of claim 1, 3 or 4, wherein said ratio T_{REG}/T_{EFF} is assigned to a probability of said patient reacting to a therapeutic comprising a CRISPR associated protein, or towards a therapeutic comprising a homologue thereof, by a cytotoxic immune response,
wherein in particular
a ratio T_{REG}/T_{EFF}<0.5 is assigned to a high risk,
a ratio 0.5≤T_{REG}/T_{EFF}<1 is assigned to a medium risk,
a ratio T_{REG}/T_{EFF}≥1 is assigned to a low risk
of said patient reacting to a CRISPR associated protein, or towards a homologue thereof, by an effector T cell response.

9. A method for preparing a preparation of T cells specifically reactive towards a CRISPR associated protein, particularly Cas9 or Cas12, more particularly towards SpCas9, or towards a homologue thereof, comprising the steps of
providing a T cell preparation; wherein in particular said T cell preparation is a preparation of regulatory T cells
introducing a nucleic acid expression construct into said T cell preparation, yielding a transgene T cell preparation,
wherein said nucleic acid expression construct encodes a T cell receptor molecule capable of specifically recognizing an HLA-presented antigen derived from a CRISPR associated protein, particularly from Cas9 or Cas12, more particularly from SpCas9, or from a homologue of such CRISPR associated protein.

10. A method for preparing a preparation of regulatory T cells specifically reactive towards a CRISPR associated protein, particularly Cas9 or Cas12, more particularly SpCas9,or towards a homologue thereof, comprising the steps of
a. providing a cell preparation comprising T cells;
b. in a first isolation step, isolating (non-activated) regulatory T cells using a set of molecular probes specific to non-activated regulatory T cells,
c. in a stimulation step, contacting said cell preparation with
an isolated CRISPR associated protein polypeptide, particularly a Cas9 or Cas12 polypeptide, more particularly SpCas9,or a homologue of such CRISPR associated protein, or
a plurality of peptides, wherein said plurality of peptides represents the amino acid sequence of said CRISPR associated protein polypeptide, particularly said Cas9 or Cas12 polypeptide, more particularly SpCas9, or said homologue thereof,
providing activated T cells in a stimulated cell preparation;
d. in a second isolation step, isolating activated regulatory T cells from said stimulated cell preparation using
a set of molecular probes specific to activated regulatory T cells, or
a set of molecular probes specific to activation-specific marker molecules;
e. in a cell proliferation step, cultivating said activated regulatory T cells,
wherein either one of the first and second isolation step is performed or both first and second isolation steps are performed.

11. The method according to any one of claim 9 or 10, wherein the T cell preparation of claim 9 is isolated using a set of molecular probes specific to non-activated regulatory T-cells or in said first isolation step of claim 10, a set of molecular probes specific to non-activated regulatory T cells is used, and wherein said set of molecular probes specific to non-activated regulatory T cells comprises ligands specific to CD3, CD4, CD25 and CD127 and/or CD137,
particularly wherein said ligands specific to CD3, CD4 or CD25 are used for positive selection, and wherein in case of a ligand specific to CD25 only cells with a high CD25 expression are selected, and wherein said ligands specific to CD127 and/or CD137 are used for negative selection.

12. The method according to any one of claim 9 or 11, wherein a transgene T cell preparation is kept under conditions of cell culture in a cell proliferation step.

13. The method according to claims 10 to 11 or claim 12, wherein IL-2 and optionally any one of resveratrol, a resveratrol analogue, a resveratrol derivative, or an mTor inhibitor are present in said cell proliferation step
wherein in particular 50 IU/ml to 5000 IU/ml, particularly 50 IU/ml to 2000 IU/ml, more particularly 200 IU/ml to 1000 IU/m of IL-2 are present in said cell proliferation step and optionally 50 nM to 150 nM, particularly 100 nM resveratrol, a resveratrol analogue, a resveratrol derivative or mTor inhibitor are present in said cell proliferation step.

14. The method according to any one of claims 10 to 13, wherein in said second isolation step,
said set of molecular probes specific to activated regulatory T cells comprises ligands specific to CD3, CD4, CD137, CD154, CD25 and CD127,
and optionally, one or more ligands specific to CD69, CD71, CD103, CD134, GARP, HLA-DR, KLRG1 or LAP, or
said set of molecular probes specific to activation-specific marker molecules comprises a ligand specific to CD137 and optionally one or more ligands specific to CD69, CD71, CD103, CD134, GARP, HLA-DR, KLRG1 or LAP,
particularly wherein in said second isolation step,
said ligands specific to CD3, CD4, CD25, CD137, CD69, CD71, CD103, CD134, GARP, HLA-DR, KLRG1 or LAP are used for positive selection, wherein in case of a ligand specific to CD25 only cells with a high CD25 expression are selected;
said ligands specific to CD127 or CD154 are used for negative selection.

15. A preparation of isolated regulatory T cells specifically reactive towards a CRISPR associated protein polypeptide, particularly a Cas9 or Cas12 polypeptide, more particularly towards SpCas9, or towards a homologue of such CRISPR associated protein, wherein said isolated regulatory T cells [each] comprise a transgenic nucleic acid sequence encoding a T cell receptor molecule capable of specifically recognizing an HLA-presented antigen derived from a CRISPR associated protein, or a preparation of isolated regulatory T cells specifically reactive towards a CRISPR associated protein polypeptide, particularly a Cas9 or Cas12 polypeptide, more particularly towards SpCas9, or towards a homologue of such CRISPR associated protein, obtained by a method according to any one of claims 10 to 16 for use in a treatment of a condition benefitting from editing a disease related DNA segment, wherein the disease is selected from human papillomavirus-related malignant neoplasm, HIV-1-infection, sickle cell disease, chronic granulomatous disease, multiple myeloma, melanoma, synovial sarcoma, myxoid/round cell liposarcoma, gastrointestinal infection, B cell leukemia, B cell lymphoma, esophageal cancer, neurofibromatosis type 1, tumors of the central nervous system, invasive bladder cancer, hormone refractory prostate cancer, metastatic renal cell carcinoma, metastatic non-small cell lung cancer, gastric carcinoma, nasopharyngeal carcinoma, T cell lymphoma, adult Hodgkin lymphoma, diffuse large B cell lymphoma, β-thalassemia, immunodysregulation polyendocrinopathy enteropathy X-linked (IPEX) syndrome, rheumatic fever, S. pyogenes-associated pharyngitis, S. pyogenes-associated pyoderma, neuroblastoma, acute myelogenous leukemia (AML), acute lymphoblastic leukemia (ALL), chronic myelogenous leukemia (CML), chronic lymphocytic leukemia (CLL), retinoblastoma, Parkinson's disease, Alzheimer's disease, muscular dystrophy, particularly Becker's muscular dystrophy, Duchenne muscular dystrophy, metabolic disease of the liver, familiar osteopetrosis, osteoporosis, osteogenesis imperfecta, Leber's congenital amaurosis, congenital hearing loss, common variable immunodeficiency (CVID), cardiomyopathy and diseases caused by viral infections, particularly by herpes virus infections, more particularly Epstein-Barr virus (EBV) infection, human cytomegalovirus (CMV) infection, herpes simplex virus infection, human immunodeficiency virus (HIV) infection and human papilloma virus (HPV) infection,
particularly wherein said preparation is administered prior to and/or concomitant with administration of a gene therapy agent comprising a CRISPR associated protein, particularly Cas9 or Cas12, more particularly SpCas9, or a homologue of such CRISPR associated protein, or of a gene therapy agent comprising a polynucleotide sequence encoding a CRISPR associated protein, particularly Cas9 or Cas12, or a homologue of such CRISPR associated protein.
